(19) United States
(12) Patent Application Publication
(10) Pub. No.: US 2020/0276181 A1
(43) Pub. Date: Sep. 3, 2020

(54) THIENOISOQUINOLINES AND THEIR DERIVATIVES FOR TARGETING TUBULIN, CH-TOG, AURORA A KINASE, TPX2, CDK5RAP2 AND/OR ASPM

(71) Applicant: VALORBEC, SEC, Montréal (CA)

(72) Inventors: Pasquale FORGIONE, Montréal (CA); Alisa Julienne PIEKNY, Lachine (CA); Kevin LAROCQUE, Montréal (CA); Dilan Boodhai JAUNKY, Montréal (CA); Fei CHEN, Pierrefonds (CA); Jiang Tian LIU, Montréal (CA)

(73) Assignee: VALORBEC, SEC, Montréal, QC (CA)

(21) Appl. No.: 16/764,370

(22) PCT Filed: Nov. 15, 2018
(86) PCT No.: PCT/CA2018/051454

§ 371 Date: May 14, 2020

Related U.S. Application Data
(63) Continuation of application No. PCT/CA2017/051473, filed on Dec. 6, 2017.
(60) Provisional application No. 62/586,553, filed on Nov. 15, 2017.
(60) Provisional application No. 62/681,093, filed on Jun. 6, 2018.

(51) Int. Cl. A61K 31/4743; A61K 31/4184; A61K 31/704; A61K 31/337; A61K 31/4375; A61K 31/165; A61K 31/09; A61K 31/365; A61P 35/00

(52) U.S. Cl. CPC A61K 31/4743; A61K 31/4184; A61K 31/704; A61K 31/337; A61P 35/00; A61K 31/165; A61K 31/09; A61K 31/365; A61K 31/4375

(57) ABSTRACT

The present disclosure relates to compounds, methods and uses thereof for targeting tubulin, ch-TOG, Aurora A kinase, TPX2, Cdk5rap2 and/or ASPM and for the treatment of cancer in a subject. For example, the compounds can comprise compounds of Formula I or a pharmaceutically acceptable salt, solvate or prodrug thereof. A, Z, R_{A}, R_{B}, R₁, R₂, R₃, R₄ and R₅ can have different values.

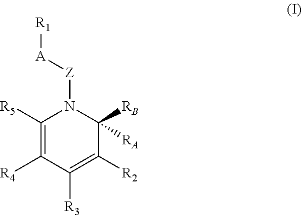

### CROSS REFERENCE TO RELATED APPLICATIONS

This is a Patent Cooperation Treaty Application claims priority to U.S. Provisional Patent Application No. 62/586,553, filed Nov. 15, 2017; U.S. Provisional Patent Application No. 62/681,093, filed Jun. 6, 2018; and International Patent Application No. PCT/CA2017/051473 filed Dec. 6, 2017. These documents are hereby incorporated by reference in their entirety.

### FIELD OF THE DISCLOSURE

This disclosure relates to thienoisoquinoline compounds and their derivatives, and more particularly to methods for targeting tubulin, ch-TOG, Aurora A kinase, TPX2, Cdk5rap2 and/or ASPM using thienoisoquinoline compounds.

### BACKGROUND OF THE DISCLOSURE

Cancer is now the leading cause of death in Canada. According to statistics from the Canadian Cancer Society, 1 out of 4 Canadians will die from the disease, and 2 out of 5 Canadians will develop cancers over their lifetime. Since the incidence of cancer is higher in people aged 50 and older, these numbers are expected to soar as the number of senior citizens in Canada increases. While the mortality rates from some cancers have decreased due to success in the clinic or through prevention (e.g. breast and prostate cancers), many aggressive, hard-to-treat cancers persist (e.g. lung, pancreatic and brain cancers). One of the main methods used to treat cancers is via the use of chemotherapies, which often has severe side-effects for patients, because most chemotherapeutic drugs also target healthy cells. Further, patients often develop resistance to drugs through various mechanisms. Combinatorial therapies are now being used to reduce side-effects and resistance, where two or more drugs are administered simultaneously or concurrently at lower doses. In addition, personalized medicine, where genetic profiling of individual tumours is used to tailor treatments more specifically to each patient, is another, more recent, treatment option. Thus, it is important to expand the repertoire of drugs to increase the number of cancers that can be treated effectively. However, current methods that are being used to search for novel anti-cancer compounds are often not successful^{[1]}. They may be too restrictive, because they search for compounds with a specific molecular target that may not be optimal, or their drug-like qualities and ease of synthesis are not considered. For example, poor quality compounds may have solubility issues, they may aggregate and require high concentrations to be effective in vivo^{[2-6]}.

A subset of successful anti-cancer drugs used to treat a wide spectrum of cancers target mitosis, which is important for cell division^{[12,13]}. One of the hallmarks shared by cancer cells is that they divide rapidly in an uncontrolled manner. The mitotic spindle is a structure that forms to align and segregate chromosomes, and to ensure that each daughter cell inherits the appropriate genetic content during division^{[14]}. If the mitotic spindle fails to attach to the chromosomes properly, then the spindle assembly checkpoint (SAC) is not satisfied and the cell will arrest and undergo apoptosis^{[14-17]}. Alternatively, chromosomes can be mis-segregated, leading to aneuploidy and mitotic catastrophe in subsequent divisions^{[18]}. As healthy somatic cells enter mitosis, two centrosomes move apart and nucleate microtubules to form a bipolar spindle that then captures the chromosomes^{[19]}. Many metastatic cancer cells have aberrant centrosomes, which are structurally or functionally defective^{[20-22}]. Since centrosomes are the main sites for nucleating microtubules, cells with aberrant centrosomes often have defective mitotic spindles, such as multipolar spindles, with defective chromosome attachment^{[23]}. Therefore, cancer cells rely on mechanisms to cluster fragmented or amplified centrosomes to form two poles^{[16,17,20-22]}. The mechanisms that cancer cells use to cluster aberrant centrosomes are not well-understood, but are attractive to target via chemotherapies because their requirement is selective to cancer cells. In support of this, several publications have described searching for compounds that specifically target centrosome clustering^{[24-29]} However, the compounds described in these papers do not achieve high efficacy and are not ideal for clinical phase trials.

Thienoisoquinoline-phenyl sulfonamide compounds have been described in U.S. Pat. No. 7,696,221 (herein incorporated by reference in its entirety) for use as ER-NF kappa B inhibitors. Synthesis of thienoisoquinolines by a 5-step linear synthesis employing a palladium-catalyzed decarboxylative cross-coupling and functionalization sequence has been reported by Chen et al.^{[11]}, herein incorporate by reference in its entirety.

### SUMMARY OF THE DISCLOSURE

In accordance to a first aspect disclosed herein, there is provided a method for targeting tubulin, ch-TOG, Aurora A kinase, TPX2, Cdk5rap2 and/or ASPM expressed in a cancer cell and selectively inhibiting growth therein, comprising exposing said cancer cell to a compound of Formula I:

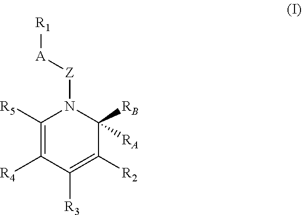

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} said C₆-C₁₂ aryl and said three- to seven-membered aromatic heterocycle being each independently unsubstituted or substituted with at least one substituent chosen from C₁-C₆ alkyl, C₁-C₆ alkoxy, C₆-C₁₂ aryl, the three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂, CF₃, CF₂H, CFH₂, F, Cl, Br, I, OH, CHO, COOH and COOR_{C}, wherein R_{C} is a C₁-C₆ alkyl,
or a pharmaceutically acceptable salt, solvate or prodrug thereof.

In accordance to another aspect disclosed herein, there is provided a method for targeting tubulin, ch-TOG, Aurora A kinase, TPX2, Cdk5rap2 and/or ASPM expressed in a cancer cell and selectively inhibiting growth therein, comprising exposing said cancer cell to a compound of Formula

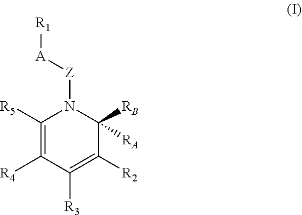

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} said C₆-C₁₂ aryl and said three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, CF₃, CF₂H, CFH₂, F, Cl, Br and I,
or a pharmaceutically acceptable salt, solvate or prodrug thereof.

According to another aspect, there is provided herein a method for targeting tubulin, ch-TOG, Aurora A kinase, TPX2, Cdk5rap2 and/or ASPM expressed in a cancer cell and selectively inhibiting growth therein, comprising exposing said cancer cell to a combination of a compound of Formula I and an anti-cancer agent and/or an anti-mitotic agent.

Another aspect herein disclosed relates to a method for targeting tubulin, ch-TOG, Aurora A kinase, TPX2, Cdk5rap2 and/or ASPM expressed in a cancer cell and inhibiting growth therein, comprising exposing said cancer cell to a synergistic combination of a compound of Formula I and an anti-cancer agent and/or an anti-mitotic agent, wherein said combination more than additively inhibits growth of said cancer cell.

In yet another aspect there is provided a use of a compound of Formula I for targeting tubulin, ch-TOG, Aurora A kinase, TPX2, Cdk5rap2 and/or ASPM expressed in a cancer cell and selectively inhibiting growth therein.

According to another aspect, there is provided a use of a combination of a compound of Formula I and an anti-cancer agent and/or an anti-mitotic agent for targeting tubulin, ch-TOG, Aurora A kinase, TPX2, Cdk5rap2 and/or ASPM expressed in a cancer cell and selectively inhibiting growth therein

According to an aspect, there is provided herein a method for selectively inhibiting growth in a cancer cell, comprising exposing the cancer cell to a compound of Formula I:

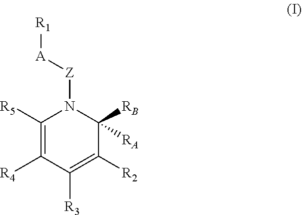

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} said C₆-C₁₂ aryl and said three- to seven-membered aromatic heterocycle being each independently unsubstituted or substituted with at least one substituent chosen from C₁-C₆ alkyl, C₁-C₆ alkoxy, C₆-C₁₂ aryl, the three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂, CF₃, CF₂H, CFH₂, F, Cl, Br, I, OH, CHO, COOH and COOR_{C}, wherein R_{C} is a C₁-C₆ alkyl,
or a pharmaceutically acceptable salt, solvate or prodrug thereof.

According to an aspect, there is provided herein a method for selectively inhibiting growth in a cancer cell, comprising exposing the cancer cell to a compound of Formula I:

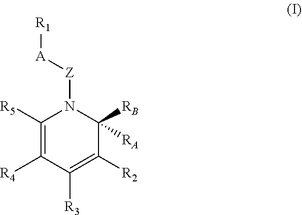

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} the C₆-C₁₂ aryl and the three- to seven-membered aromatic heterocycle being each independently unsubstituted or substituted with at least one substituent chosen from C₁-C₆ alkyl, C₁-C₆ alkoxy, C₆-C₁₂ aryl, the three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂ CF₃, CF₂H, CFH₂, F, Cl, Br and I, OH, CHO,
or a pharmaceutically acceptable salt, solvate or prodrug thereof.

According to another aspect, there is provided herein a method for disrupting centrosome integrity, preventing and/or reducing centrosome clustering, declustering centrosomes, regulating centrosome clustering and/or altering microtubule dynamics including microtubule depolymerization in a cancer cell, comprising exposing the cancer cell to a compound of Formula I.

According to yet another aspect, there is provided herein a method for selectively inhibiting growth in a cancer cell, comprising exposing the cancer cell to a combination of a compound of Formula I and an anti-cancer agent and/or an anti-mitotic agent.

According to another aspect, there is provided herein a method for inhibiting growth in a cancer cell, comprising exposing the cancer cell to a synergistic combination of a compound of Formula I and an anti-cancer agent and/or an anti-mitotic agent, wherein the combination more than additively inhibits growth of the cancer cell.

According to another aspect, there is provided herein a method for increasing selectivity of an anti-cancer agent and/or an anti-mitotic agent to a cancer cell, comprising exposing the cancer cell with a compound of Formula I and the anti-cancer agent and/or the anti-mitotic agent.

According to another aspect, there is provided herein a method of treating a cancer in a subject, comprising administering to the subject an effective amount of a compound of Formula I.

According to another aspect, there is provided herein a method of treating a cancer in a subject, comprising administering to the subject an effective amount of a combination of a compound of Formula I and an anti-cancer agent and/or an anti-mitotic agent.

According to a further aspect, there is provided herein a use of a compound of Formula I for selectively inhibiting growth in a cancer cell.

According to another aspect, there is provided herein a use of a compound of Formula I for disrupting centrosome integrity, preventing and/or reducing centrosome clustering, declustering centrosomes, regulating centrosome clustering and/or altering microtubule dynamics including microtubule depolymerization in a cancer cell.

According to yet another aspect, there is provided herein a use of a combination of a compound of Formula I and an anti-cancer agent and/or an anti-mitotic agent for selectively inhibiting growth in a cancer cell.

According to another aspect, there is provided herein a use of a compound of Formula I for the treatment of cancer in a subject.

According to another aspect, there is provided herein a use of a combination of a Formula I and an anti-cancer agent and/or an anti-mitotic agent for the treatment of cancer in a subject.

According to another aspect, there is provided herein a use of a compound of Formula I in combination with an anti-cancer agent and/or an anti-mitotic agent for increasing selectivity of the anti-cancer agent and/or the anti-mitotic agent to a cancer cell.

According to a further aspect, there is provided herein a compound of Formula I:

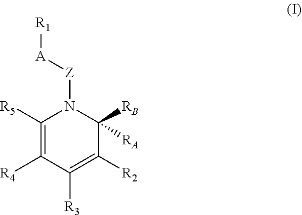

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} said C₆-C₁₂ aryl and said three- to seven-membered aromatic heterocycle being each independently unsubstituted or substituted with at least one substituent chosen from C₁-C₆ alkyl, C₁-C₆ alkoxy, C₆-C₁₂ aryl, three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂ CF₃, CF₂H, CFH₂, F, Cl, Br and I, OH, CHO,
or a pharmaceutically acceptable salt, solvate or prodrug thereof.

According to a further aspect, there is provided herein a compound of Formula IA:

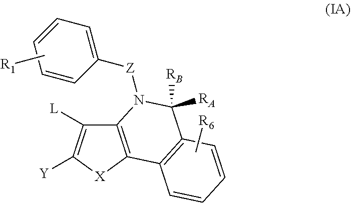

wherein
L is H, C₁-C₃ alkyl, C₁-C₃ alkoxy, C₁-C₃ thioalkyl, C₁-C₃ haloalkyl, CN, CF₃, CF₂H, CFH₂, F, Cl, Br or I;
X is S, O, NR₇ or NH;
Y is F, Cl, Br, I, H, CH₃, CF₃, CHF₂, CF₂H or CN;
Z is SO₂, CO or CH₂;
R_{A} and R_{B} are each independently H, Me, Et, CF₃, CF₂H, CFH₂, F or Cl;
R₁ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I;
R₆ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I; and
R₇ is C₁-C₆ alkyl, C₁-C₆ haloalkyl, C₁-C₆ sulfonylakyl, C₁-C₆ alkylamino, CF₃, CF₂H, CFH₂, a C₆-C₁₂ aryl or a three- to seven-membered aromatic heterocycle,
L, R_{A}, R_{B}, R₁, R₆, the C₆-C₁₂ aryl and the three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ haloalkyl, F, Cl, Br and I,
or a pharmaceutically acceptable salt, solvate or prodrug thereof.

According to a further aspect, there is provided herein a compound chosen from:

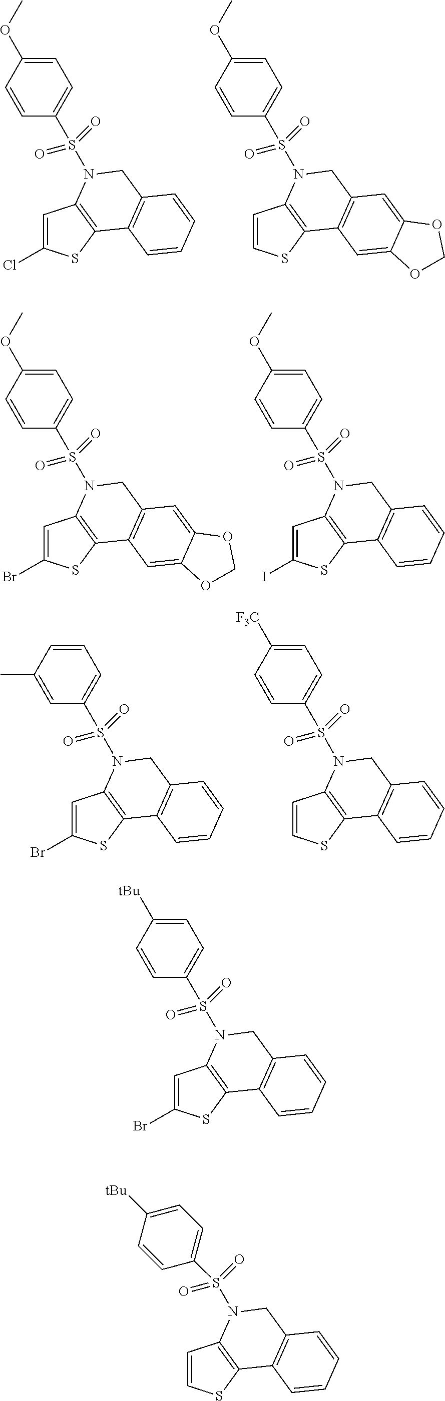

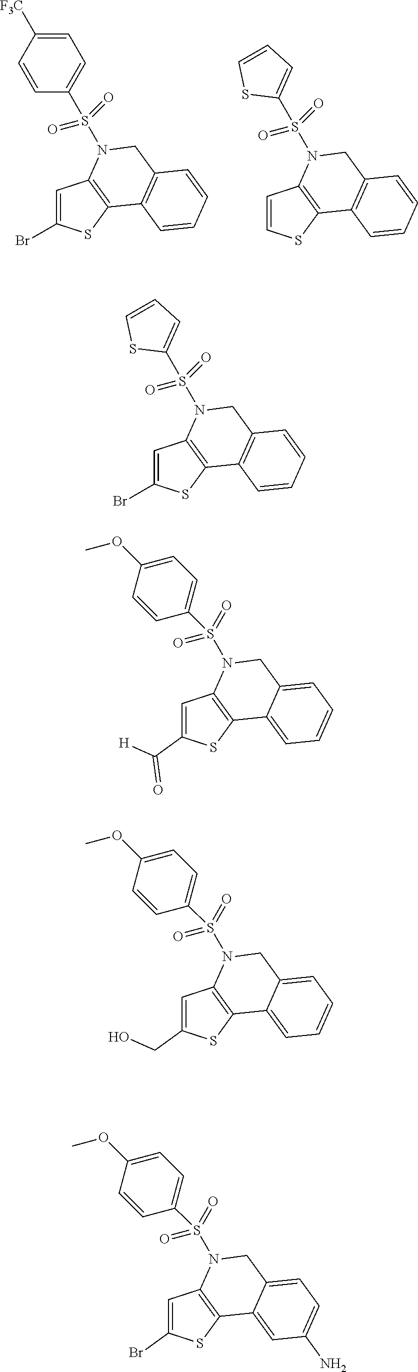

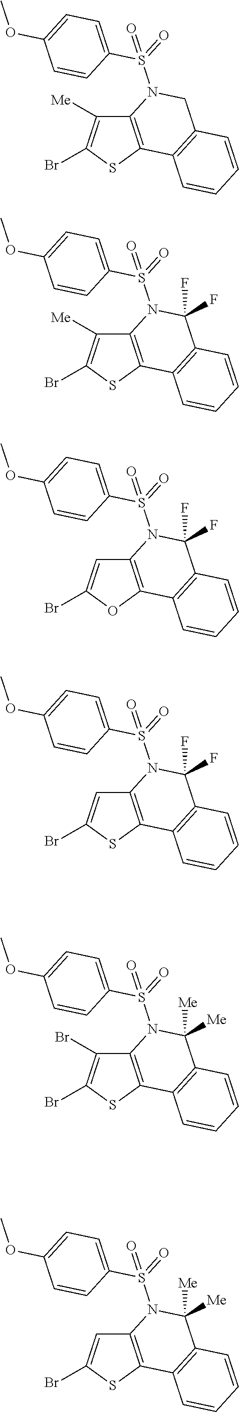

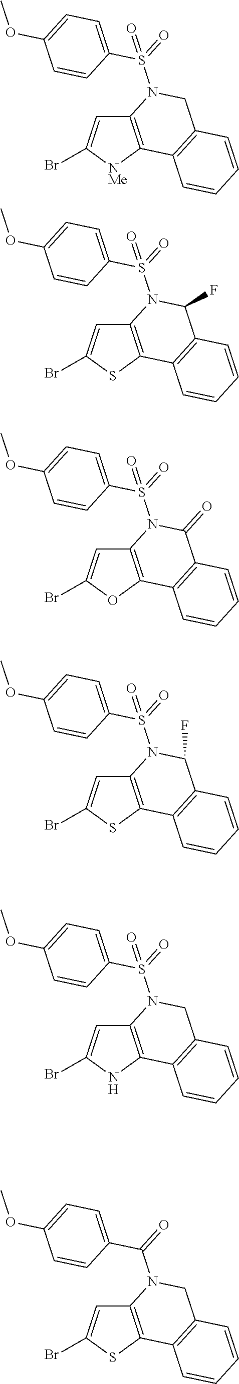 [SMILES: COc1ccc(S(=O)(=O)N2Cc3ccccc3-c3c2cc(Br)n3C)cc1 ; COc1ccc(S(=O)(=O)N2c3cc(Br)sc3-c3ccccc3[C@@H]2F)cc1 ; COc1ccc(S(=O)(=O)n2c(=O)c3ccccc3c3oc(Br)cc32)cc1 ; COc1ccc(S(=O)(=O)N2c3cc(Br)sc3-c3ccccc3[C@H]2F)cc1 ; COc1ccc(S(=O)(=O)N2Cc3ccccc3-c3[nH]c(Br)cc32)cc1 ; COc1ccc(C(=O)N2Cc3ccccc3-c3sc(Br)cc32)cc1]

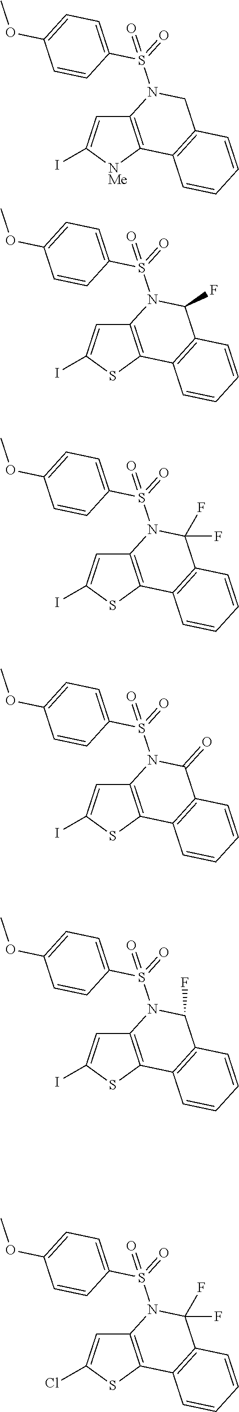 [SMILES: COc1ccc(S(=O)(=O)N2Cc3ccccc3-c3c2cc(I)n3C)cc1 ; COc1ccc(S(=O)(=O)N2c3cc(I)sc3-c3ccccc3[C@@H]2F)cc1 ; COc1ccc(S(=O)(=O)N2c3cc(I)sc3-c3ccccc3C2(F)F)cc1 ; COc1ccc(S(=O)(=O)n2c(=O)c3ccccc3c3sc(I)cc32)cc1 ; COc1ccc(S(=O)(=O)N2c3cc(I)sc3-c3ccccc3[C@H]2F)cc1 ; COc1ccc(S(=O)(=O)N2c3cc(Cl)sc3-c3ccccc3C2(F)F)cc1]

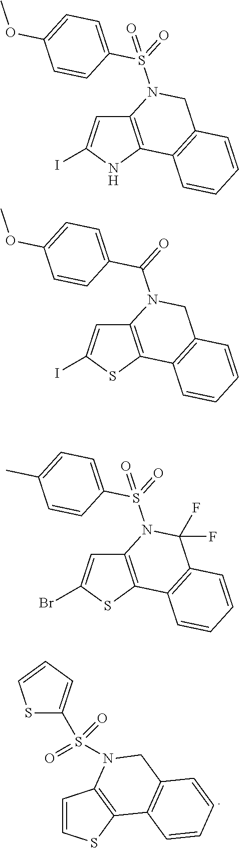 [SMILES: COc1ccc(S(=O)(=O)N2Cc3ccccc3-c3[nH]c(I)cc32)cc1 ; COc1ccc(C(=O)N2Cc3ccccc3-c3sc(I)cc32)cc1 ; Cc1ccc(S(=O)(=O)N2c3cc(Br)sc3-c3ccccc3C2(F)F)cc1 ; O=S(=O)(c1cccs1)N1Cc2ccccc2-c2sccc21]

According to a further aspect, there is provided herein a compound of Formula IB:

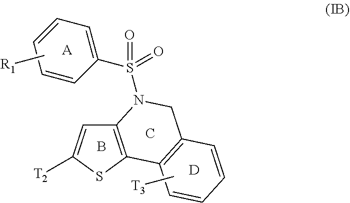

TABLE 1

| | T₁ | T₂ | T₃ |
|---|---|---|---|
| C39 | 3-Me | H | H |
| C71 | 4-Me | Ph | H |
| C74 | 4-Me | Br | H |
| C75 | 4-OMe | Br | H |
| C90 | 4-OMe | H | H |
| C91 | 4-Me | H | H |
| C93 | 3-Me | Br | H |
| C108 | 4-CF₃ | H | H |
| C200 | 4-CF₃ | Br | H |
| C201 | 4-tBu | Br | H |
| C207 | 4-OMe | I | H |
| C208 | 4-OMe | Cl | H |

According to a further aspect, there is provided herein a compound of Formula IC:

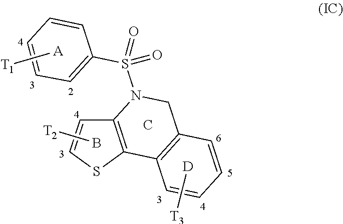

TABLE 2a

| T₁ | T₂ | T₃ |
|---|---|---|
| 4-Me | H | H |
| 3-Me | H | H |
| 2-Me | H | H |
| 4-OMe | H | H |
| 4-F | H | H |
| 4-Me | 3-Br | H |
| 4-OMe | 3-Br | H |
| 4-Me | 3-Benzyl | H |
| 4-Me | 3-Toluenyl | H |
| 4-Me | 3-Naphthalenyl | H |
| 4-Me | 3-(4-Methoxylphenyl) | H |
| 4-Me | 3-(3-Methoxylphenyl) | H |
| 4-Me | 3-(4-ethyl benzoate) | H |
| 4-Me | 3-(3-ehtyl benzoate) | H |
| 4-Me | 3-(4-fluorophenyl) | H |
| 4-Me | 3-(4-benzonitrile) | H |
| 4-Me | 3-(4-trifluoromethylphenyl) | H |
| 4-Me | 3-pyridinyl | H |
| 4-OMe | 3-Cl | H |
| 4-OMe | 3-I | H |
| 3-Me | 3-Br | H |
| 4-CF₃ | H | H |
| 4-CF₃ | 3-Br | H |
| 4-tBu | 3-Br | H |
| 4-OMe | 3-CHO | H |
| 4-OMe | 3-CH₂OH | H |
| 4-OMe | 3-CH₂OCH₃ | H |
| 4-OMe | 3-COCH₃ | H |
| 4-OMe | 3-Br | 4,5-dioxoyl |
| 4-OMe | 3-Br | 3-NO₂ |
| 4-OMe | 3-Br, 4-Methyl | H |
| 4-OMe | 3-Br | 3-NH₂, 6-Br |
| Thiophene ring | 3-Br | H |
| 4-OMe | 3,4-di-Me | H |
| 4-OMe | 3-CN, 4-Me | H |
| 4-OMe | 3-Br | 3-Br |
| 4-OMe | 3-Br | 3-NH₂ |
| 4-OMe | 3-H | 6-NO₂ |
| 4-OMe | 3-H | 4-NH₂ |
| 4-OMe | 3-Br | 4-NH₂ |
| 4-OMe | 3-Br | 6-NH2 |
| 4-OMe | 3-OH | 6-NH2 |
| 4-OMe | 3-Br | 4-NO₂ |
| 4-OMe | 3-Br | 6-NO₂ |
| 4-OMe | 3-Br | 3-OH |
| 4-OMe | 3-H | 4-NO₂ |
| 4-OMe | 3-NO₂ | H |
| 4-OMe | 3-Me | H |
| 4-OMe | 3-NH₂, 4-Me | H |
| 4-OMe | 3-CHO, 4-Me | H |
| 4-OMe | 3-OH, 4-Me | H |
| 4-OMe | 3-OH | H |
| 4-OMe | 3-NH₂ | H |
| 4-OMe | 3-CF₃ | H |
| 4-OMe | 3-F | H |
| 4-OMe | 3-OMe | H |
| 4-OMe | 3-CN | H |
| 4-OEt | 3-Br | H |
| 4-OiPr | 3-Br | H |
| 4-NMe₂ | 3-Br | H |
| 4-OMe | 3-Br, 4-Br | H |

According to a further aspect, there is provided herein a compound of Formula ID:

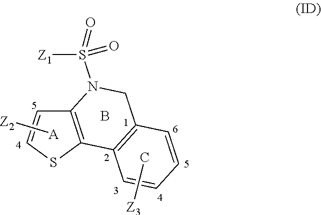

TABLE 2b

| Z₁ | Z₂ | Z₃ |
|---|---|---|
| 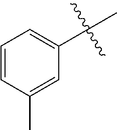 [SMILES: *c1cccc(C)c1] | H | H |
| 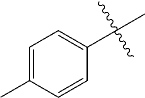 [SMILES: *c1ccc(C)cc1] | 5-C₆H₆ | H |
| 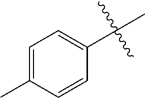 [SMILES: *c1ccc(C)cc1] | 5-Anisole | H |
| 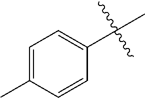 [SMILES: *c1ccc(C)cc1] | 5-Bromo | H |
| 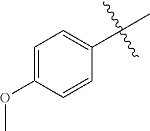 [SMILES: *c1ccc(OC)cc1] | 5-Bromo | H |
| 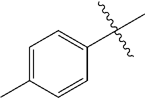 [SMILES: *c1ccc(C)cc1] | 5-Naphthalene | H |
| 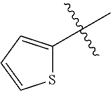 [SMILES: *c1cccs1] | H | H |
| 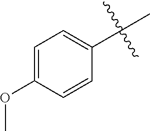 [SMILES: *c1ccc(OC)cc1] | H | H |
| 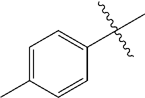 [SMILES: *c1ccc(C)cc1] | H | H |
| 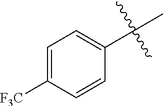 [SMILES: *c1ccc(C(F)(F)F)cc1] | H | H |
| 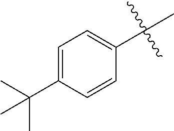 [SMILES: *c1ccc(C(C)(C)C)cc1] | H | H |
| 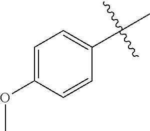 [SMILES: *c1ccc(OC)cc1] | 4-CH₃ | H |
| 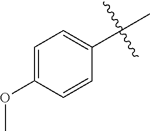 [SMILES: *c1ccc(OC)cc1] | 4-CH₃, 5-Chloro | H |
| 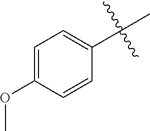 [SMILES: *c1ccc(OC)cc1] | H | 3-NO₂ |
| 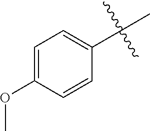 [SMILES: *c1ccc(OC)cc1] | 5-Bromo | 3-NO₂ |
| 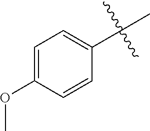 [SMILES: *c1ccc(OC)cc1] | H | 3-NH₂ |
| 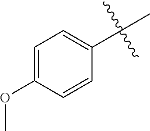 [SMILES: *c1ccc(OC)cc1] | 5-Bromo | 3-NH₂, 6-Bromo |
| 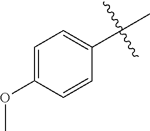 [SMILES: *c1ccc(OC)cc1] | 4-CH₃, 5-CHO | H |
| 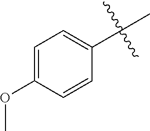 [SMILES: *c1ccc(OC)cc1] | 4-CH₃, 5-Bromo | H |
| 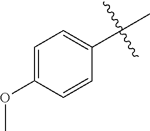 [SMILES: *c1ccc(OC)cc1] | 5-CHO | H |
| 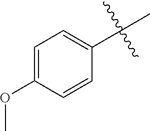 [SMILES: *c1ccc(OC)cc1] | 5-COOH | H |
| 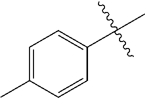 [SMILES: *c1ccc(C)cc1] | 5-CHO | H |
| 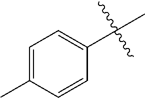 [SMILES: *c1ccc(C)cc1] | 5-COOH | H |
| 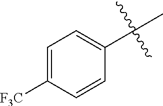 [SMILES: *c1ccc(C(F)(F)F)cc1] | 5-Bromo | H |
| 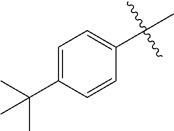 [SMILES: *c1ccc(C(C)(C)C)cc1] | 5-Bromo | H |
| 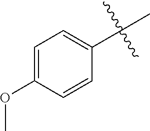 [SMILES: *c1ccc(OC)cc1] | 5-Iodo | H |
| 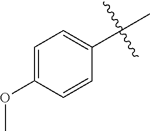 [SMILES: *c1ccc(OC)cc1] | 5-Chloro | H |
| 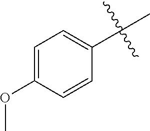 [SMILES: *c1ccc(OC)cc1] | H | 4,5-CH₂O₂ |
| 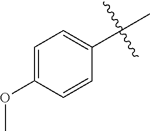 [SMILES: *c1ccc(OC)cc1] | 5-Bromo | 4,5-CH₂O₂ |

### BRIEF DESCRIPTION OF THE FIGURES

Further features and advantages of the disclosure will become more readily apparent from the following description of specific embodiments as illustrated by way of examples in the appended figures wherein:

FIG. 1A shows that Compound 75 (C75) selectively causes toxicity in cancer cells, and FIGS. 1B, C and D show that C75 selectively causes mitotic arrest in cancer cells and disrupts spindle morphology. FIG. 1E shows that Compound 140 (C140) also causes spindle defects. FIG. 1A) is a line graph showing changes in the percentage of viable HFF-1, HeLa, A549 and HCT 116 cells after treatment with a range of C75 concentrations for 3 population doubling times (N=3). The inhibitory concentration reducing viability of 50% of the population (IC₅₀) is shown by the dotted line. FIG. 1B) shows bright-field images of fields of view of HeLa cells treated with DMSO or 500 nM C75 for 8 hours. There were more rounded mitotic cells after C75 treatment in comparison to the control. FIG. 1C) is a line graph showing the percentage of HeLa and HFF1 cells in mitosis after treatment with various concentrations of C75 for 8 hours. The bars indicate standard deviation. FIG. 1D) is a series of images showing cells that were fixed and co-stained for tubulin (microtubules) and DAPI (DNA). Treatment of HeLa cells with 300 nM C75 caused the mitotic spindles to be disorganized, while they were not affected in HFF1 cells. Treatment of 750 nM C75 caused complete fragmentation of the mitotic spindles in HeLa cells, while they were disorganized in HFF1 cells. The scale bar for the cells is 10 μm. FIG. 1E) shows images of A549 cells 4 hours after treatment with 300 nM C140, co-stained for DAPI and tubulin. FIG. 1F) is a cartoon schematic showing the key features of a metaphase cell.

FIGS. 2A, 2B, 2C, 2D, 2E, 2F, 2G, 2H, 2I, 2J, 2K 2L and 2M show that C75 selectively causes mitotic arrest in cancer cells and targets centrosomes. FIG. 2A) shows bar graphs (left) of changes in the proportion of HeLa, A549 and HCT 116 cells in different phases of the cell cycle after treatment with a range of concentrations of C75 for 8 hours, as determined by flow cytometry. For each condition, 20,000 cells were counted per replicate (N=3). Line graphs (right) were made to better illustrate the relative changes in the cell cycle phases for the different treatments. Bars show standard deviation. The asterisks indicate the first statistically significant change in the G0/G1 and G2/M populations determined by a two-way ANOVA with a post-hoc Tukey's multiple comparisons test using 95% confidence intervals. FIG. 2B) is a line graph showing the percentage of HCT116 (p53−/−) cells, A549 cells, HeLa cells and H1299 cells in mitosis after treatment with various concentrations of C75 for one population doubling time. All of the cell lines show an increase in the proportion of mitotic cells after treatment with 200 nM C75. The bars indicate standard deviation. FIG. 2C) is a series of images showing HFF1, H1299, MCF10A and MCF7 cells that were fixed and co-stained for tubulin (microtubules) and DAPI (DNA) after 6-8 hours of treatment with 300 nM of C87 (inactive derivative) or C75 as indicated. While the mitotic spindles were not dramatically altered by C75 in HFF1 cells, they were monopolar (as shown) or fragmented in the other cell types. FIG. 2D) are images showing fixed HeLa, BT-549, A549 and HCT116 (p53−/−) cells that were fixed and co-stained for tubulin (microtubules) and DAPI (DNA) after 6-8 hours of treatment treated with 300 nM of C87 (less active derivative) or C75 as indicated. The mitotic spindles were more strongly fragmented after C75 treatment in these cells vs. the cells in FIG. 2C). FIG. 2E) are images showing fixed HFF1, HeLa, A549 and HCT 116 cells co-stained for γ-tubulin (spindle poles; white) and ACA (centromeres; light grey) 4 hours after treatment with 300 nM C87 (control) or 300 nM C75. Cartoon schematics show examples of the different spindle phenotypes, including bipolar with aligned chromosomes (top), bipolar with misaligned chromosomes (upper middle), multipolar/fragmented (lower middle), or monopolar (bottom), and the proportion of that phenotype is shown in the top right corner of each image. FIG. 2F) is a bar graph showing the proportion of the different spindle phenotypes for HFF1, HeLa, A549 and HCT 116 cells treated as in FIG. 2E) (n=1355; N=3). Bars show standard deviation. FIG. 2G) shows a schematic of how HeLa cells in metaphase were treated with C75 or the tubulin-targeting drug nocodazole for 5 minutes, then the C75 and nocodazole drugs were washed out and cells were imaged after 40 minutes. FIG. 2H) shows images of fixed HeLa cells treated as shown in 2G), co-stained for tubulin (microtubules) and DAPI (DNA). While mitotic spindles recovered bipolarity after removing nocodazole, they remained multipolar after removing C75. FIG. 2I) images show examples of fixed HeLa cells co-stained for DAPI and tubulin (white) after treatment with 500 nM C75 or colchicine as in 2G (n=527; N=3). FIG. 2J) is a bar graph showing the proportion of cells with monopolar (black), bipolar (dark grey) or multipolar (light grey) spindles. Bars show standard deviation. FIG. 2K) is a series of time-lapse images of a live HeLa cell expressing GFP:tubulin after treatment with C75 as shown in 2G). FIG. 2L) Timelapse images show live HeLa cells stained with SiR-tubulin (microtubules in black) after treatment with C75, colchicine or control (DMSO) as indicated. Drugs were not washed out. The times are shown in the bottom right corner of each image. The scale bar for all cells is 10 μm. FIG. 2M) is a schematic showing the putative target for C75. Healthy cells have two centrosomes that separate and form a bipolar spindle, while cancer cells have aberrant centrosomes that cluster to form ‘pseudo’ bipolar spindles, and C75 may target this process and their integrity.

FIGS. 3A, 3B, 3C, 3D and 3E show that C75 enhances the efficacy and selectivity of tubulin-targeting drugs for cancer cells. FIG. 3A) is a line graph showing cytotoxicity of HeLa cells after treatment with varying concentrations of C75+/−a subthreshold dose of paclitaxel (Taxol™; 3 nM). The IC₅₀ is shown by the dotted line. Bars show standard deviation. FIG. 3B) is a line graph showing the percentage of mitotic HeLa or HFF1 cells treated with a range of paclitaxel concentrations+/−C75 as indicated. The bars indicate standard deviation. FIG. 3C) is a series of bar graphs showing the proportion of HCT 116 cells with bipolar aligned (grey), bipolar misaligned (dark grey), multipolar/fragmented (light grey, top portion of each bar) or monopolar (black) spindles 7 hours after treatment with control (DMSO), varying concentrations of paclitaxel or C75, or both as indicated (n=691; N=3). The bars show standard deviation. The combination treatments were compared to paclitaxel and C75 on their own by calculating ratios varying from the predicted ratio, and found to synergize at 100 and 200 nM. FIG. 3D) is bar graph showing the average distance between centrosome fragments in HCT116 cells after treatments as shown. While centrosome fragments moved closer together with increased concentrations of paclitaxel, they moved further apart with increased concentrations of C75 (n=105; N=3). FIG. 3E) is a series of images of fixed HCT116 cells stained for tubulin (microtubules) and DAPI (DNA), demonstrating the differences in the distance between spindle fragments. FIG. 3F) is a line graph showing the percentage of mitotic HeLa or HFF1 cells treated with a range of nocodazole concentrations+/−C75 as indicated. The bars indicate standard deviation. FIG. 3G) is a series of images showing fixed HeLa cells after treatment with C75+/− nocodazole co-stained for tubulin, γ-tubulin and DNA (DAPI). A dotted line shows the outline of the cells. Adding nocodazole to cells treated with 300 nM C75 caused mitotic spindle phenotypes to worsen and appear more similar to those after treatment with 600 nM C75. The scale bar for all cells is 10 μm. FIG. 3H) is a bar graph showing the percentage of spindle phenotypes observed in HeLa cells treated with nocodazole or C75, or both for 20 minutes (n>15 per treatment).

FIGS. 4A, 4B, 4C, 4D, 4E, and 4F show that another microtubule depolymerizing drug called colchicine increases the efficacy of C75 in cancer cells, and that C75 causes different spindle phenotypes compared to colchicine. In FIGS. 4A), 4B), and 4C) line graphs show the cytotoxicity of HeLa (FIG. 4A), A549 (FIG. 4B) and HCT116 (FIG. 4C) cells as determined by the IC₅₀ for viability (dotted lines) after treatment with C75 (triangles), colchicine (circles) or a combination of C75 and colchicine (sub-threshold doses of 3 nM, 9 nM and 3 nM, respectively; squares). FIG. 4D) shows bar graphs of the proportion of HeLa cells with bipolar (dark grey) or multipolar/fragmented (light grey) spindles after 5 hours of treatment with varying concentrations of colchicine or C75, or both as indicated (n=2939; N=3). Bars show standard deviation. FIG. 4E) is a series of images of fixed HeLa cells stained for tubulin (microtubules) and DAPI (DNA) to show the different phenotypes that were observed after treatment with DMSO, colchicine or C75, respectively. Spindle fragmentation only occurred after extensive microtubule depolymerization in colchicine-treated cells, while the spindle poles fragmented prior to changes in microtubules after C75 treatment. The scale bar for all cells is 10 μm. In FIG. 4F), bar graphs show changes in the proportion of HCT 116 cells with the different spindle phenotypes as in FIG. 4D (n=2368; N=3). Significant changes were observed in the proportion of multipolar cells at 300 and 400 nM C75 in combination with a subthreshold dose of colchicine (20 nM). Bars indicate standard deviation.

FIGS. 5A, 5B, 5C, 5D, 5E, 5F, 5G, 5H, 5I, 5J, 5K, 5L and 5M show how C75 disrupts or reduces the growth of HeLa, HCT116 and A549 spheroids. FIG. 5A) images show HeLa spheroids over time (3 days) before and after treatment with control (1 μM C87; n=10), 250 nM (n=6) or 500 nM (n=10) of C75. The scale bar is 100 μm. FIG. 5B) is a series images of HeLa spheroids after three days of treatment with control (500 nM C87) or 500 nM C75. The spheroids were stained for fluorescein diacetate (to detect live cells) and propidium iodide (to detect dead cells). The majority of HeLa cells were dead after C75 treatment. The scale bar is 100 μm. FIG. 5C) is a line graph showing the change in spheroid area (μm²) for each day and treatment as indicated. Bars show standard deviation and the asterisks are p<0.05 as determined by the student's t test. FIG. 5D) images show HeLa spheroids over 3 days before and after treatment with empty nanoparticles (control; n=3) or 250 nM of nanoparticle-encapsulated C75 (n=3). The scale bar is 100 μm. FIG. 5E) A line graph shows the change in spheroid area (μm²) for each day and treatment as indicated. Bars show standard deviation and the asterisks are p<0.05 as determined by the student's t test. FIG. 5F) is a series of images of HCT116 spheroids treated with control (1 μM C87; n=10), 500 nM C75 (n=10) and 1 μM C75 (n=10). FIG. 5G) is a series images of HCT116 spheroids after six days of treatment with control (1 μM C87) or 1 μM C75. The spheroids were stained for fluorescein diacetate (live cells) and propidium iodide (dead cells). The HCT116 spheroids were much smaller after C75-treatment, but still contained live cells. FIG. 5H) is a line graph showing the growth in surface area (%) of HCT116 spheroids treated with control (1 μM C87), 500 nM C75 and 1 μM C75 after six days. FIG. 5I) is a series of images of A549 spheroids treated with control (1 μM C87, n=6), 500 nM C75 (n=6), and 1 μM C75 (n=6) over 5 days. The scale bar is 100 μm. FIG. 5J) is a series of images of A549 spheroids after six days of treatment with control (750 nM C87) or 1 μM C75. The spheroids were stained for fluorescein diacetate (live cells) and propidium iodide (dead cells). As can be seen, C75 regressed the growth of the A549 spheroid and there were fewer live cells within the C75-treated spheroid in comparison to control. In FIG. 5K) a line graph shows the change in spheroid area (μm²) for each day and treatment as indicated. Bars show standard deviation and the asterisks are p<0.05 as determined by the student's t test. FIG. 5L) images show A549 spheroids over 6 days before and after treatment with empty nanoparticles (control; n=3) or 500 nM of nanoparticle-encapsulated C75 (n=3). The scale bar is 100 μm. FIG. 5M) is a line graph shows the change in spheroid area (μm²) for each day and treatment as indicated. Bars show standard deviation and the asterisks are p<0.05 as determined by the student's t test.

FIG. 6 is a series of timelapse images of HeLa cells expressing GFP-tagged ch-TOG (in black), treated with DMSO (control), 100 nM C75, 300 nM C75 or 50 nM colchicine. The centrosomes collapse together in the C75-treated cells more quickly vs. colchicine treatment and ch-TOG becomes enriched on the centrosomes vs. control or in cells treated with colchicine.

FIG. 7A) is a series of images of HeLa cells and HeLa cells with lower levels of endogenous ch-TOG (ch-TOG RNAi), stained for DAPI (DNA) and tubulin (microtubules), and treated with control, 200 nM C75 or 20 nM colchicine. FIG. 7B) is a bar graph showing the proportion of phenotypes of the HeLa cells observed in the different conditions listed in 7A) (bipolar spindles in light grey; bipolar spindles with misaligned chromosomes in dark grey; multipolar/fragmented spindles in hatched lines; monopolar spindles in black). While colchicine has a synergistic effect on the severity of spindle phenotypes (e.g. multipolar spindles) when treated in combination with ch-TOG RNAi, C75 has an additive effect.

FIG. 8A) is a series of images of HeLa cells with lower levels of endogenous MCAK (MCAK RNAi), stained for DAPI (DNA) and tubulin (microtubules), and treated with 300 nM C75, 30 nM colchicine or control. FIG. 8B) is a bar graph showing the proportion of spindle phenotypes in HeLa cells treated as in 8A). It was previously reported that ch-TOG RNAi multipolar spindle phenotypes are suppressed by MCAK RNAi, and MCAK RNAi suppresses the multipolar spindle phenotypes caused by C75, but not colchicine.

FIG. 9A) is a series of images of HCT116 cells with lower levels of endogenous MCAK (MCAK RNAi), stained for DAPI (DNA) and tubulin (microtubules), and treated with 300 nM C75 or control. FIG. 9B) is a bar graph showing the proportion of spindle phenotypes in HCT116 cells treated as in 9A). The multipolar spindle phenotypes caused by C75 are suppressed by MCAK RNAi.

FIG. 10 is a graph showing microtubule polymerization in vitro in the presence of 200 nM C75 or DMSO (control). Bars show SEM. Microtubules fail to polymerize in the presence of C75, but not in control samples, showing that tubulin could be a target of C75 in vitro.

FIG. 11A) is a series of images of HCT116 spheroids treated with control (DMSO), 10 nM paclitaxel, 250 nM C75 or both for 6 days. At these threshold concentrations of paclitaxel and C75, spheroids failed to grow with either drug alone, but regressed in size when treated with both in combination. The scale bar for the images is 100 μm. FIG. 11B) shows a line graph showing the change in spheroid surface area over time for the different treatments as indicated. As shown in the images, adding paclitaxel and C75 in combination led to regression in spheroid size compared to each treatment on its own.

FIG. 12A) shows images of A549 cells after treatment with TPX2 RNAi, co-stained for DAPI (to visualize DNA) and tubulin. A549 cells with lower levels of endogenous TPX2 displayed mitotic arrest and spindle phenotypes similar to what was observed for higher concentrations of C75. FIG. 12B) shows images of A549 cells 4 hours after treatment with 300 nM Alisertib (Aurora A kinase inhibitor), co-stained for DAPI and tubulin. A549 cells treated with Alisertib displayed mitotic arrest and spindle phenotypes similar to what was observed for C75.

### DETAILED DESCRIPTION OF THE DISCLOSURE

Accordingly, a class of anti-cancer compounds with potential for clinical use has been identified. By synthesizing compounds with a quinoline scaffold that already has been proven to have successful drug-like properties^{[7-11]}, it has been made possible to overcome limitations related to quality. These compounds appear to have a unique mechanism of action in comparison to known anti-cancer drugs, by targeting a process that occurs uniquely in cancer cells, making the compounds selective for cancer cells. In addition, these compounds enhance the selectivity of other anti-cancer drugs, making them suitable for use in combinatorial therapies.

### I. Definitions

As used herein, the term “ch-TOG” (colonic and hepatic tumor overexpressed gene protein), or “CKAP5” (cytoskeleton associated protein 5) refers to a microtubule polymerase that plays a role in bipolar mitotic spindle assembly and that is overexpressed in certain cancer cells such as for example colorectal adenocarcinoma cells. ch-TOG includes, without limitation, all known ch-TOG molecules, including human, naturally occurring variants as well as Uni-ProtKB ID of Q14008, herein incorporated by reference in its entirety.

As used herein “Aurora A kinase” means an enzyme that regulates the function of multiple proteins that control mitotic spindle assembly. Aurora A kinase is differentially expressed in certain cancers including breast, colorectal and lung cancer cells. Aurora A kinase includes without limitation, all known Aurora A kinase molecules, including human, naturally occurring variants as well as Uni-ProtKB ID of 014965, herein incorporated by reference in its entirety.

The term “TPX2” as used herein means a protein that mediates microtubule nucleation from the centrosomes and recruits Aurora A kinase. TPX2 is differentially expressed in certain cancers such as gastric cancer. TPX2 includes without limitation, all known TPX2 molecules, including human, naturally occurring variants as well as Uni-ProtKB ID of Q9ULW0, herein incorporated by reference in its entirety.

The term “tubulin” as used herein means alpha or beta protein that forms heterodimers to make microtubules. Tubulin also includes gamma tubulin, which forms the gamma-tubulin ring complex that nucleates microtubules. Tubulin includes without limitation, all known tubulin molecules, including human, naturally occurring variants as well as Uni-ProtKB ID of Q71U36, P07437 or P23258, herein incorporated by reference in its entirety.

The term “Cdk5rap2” as used herein means CDK5 Regulatory Subunit Associated Protein 2. Cdk5rap2 includes without limitation, all known Cdk5rap2 molecules, including human, naturally occurring variants as well as Uni-ProtKB ID of Q96SN8, herein incorporated by reference in its entirety.

The term “ASPM” as used herein means Abnormal spindle-like microcephaly-associated protein. ASPM includes without limitation, all known ASPM molecules, including human, naturally occurring variants as well as Uni-ProtKB ID of Q81ZT6, herein incorporated by reference in its entirety. ASPM is an ortholog of the Drosophila melanogaster abnormal spindle (asp) gene.

As used herein, the phrase “targeting tubulin, ch-TOG, Aurora A kinase, TPX2, Cdk5rap2 and/or ASPM” means that a compound of the present disclosure that binds, for example selectively, one or more of tubulin, ch-TOG, Aurora A kinase, TPX2, Cdk5rap2 and/or ASPM expressed in a cancer cell, and causes dysregulation of mitotic spindle assembly leading to growth inhibition of the cancer cell.

The expression “compound(s) of the present disclosure” as used in the present document refers to compounds of formulae I and IA presented in the present disclosure, isomers thereof, such as stereoisomers (for example, enantiomers, diastereoisomers, including racemic mixtures) or tautomers, or to pharmaceutically acceptable salts, solvates, hydrates and/or prodrugs of these compounds, isomers of these latter compounds, or racemic mixtures of these latter compounds. The expression “compound(s) of the present disclosure” also refers to mixtures of the various compounds or variants mentioned in the present paragraph.

It is to be understood that the present disclosure includes isomers, racemic mixtures, pharmaceutically acceptable salts, solvates, hydrates and prodrugs of compounds described therein and mixtures comprising two or more of such compounds.

The compounds of the disclosure may have at least one asymmetric centre. Where the compounds disclosed herein possess more than one asymmetric centre, they may exist as diastereomers. It is to be understood that all such isomers and mixtures thereof in any proportion are encompassed within the scope of the present disclosure. It is to be understood that while the stereochemistry of the compounds of the present disclosure may be as provided for in any given compound listed herein, such compounds of the disclosure may also contain certain amounts (for example less than 30%, less than 20%, less than 10%, or less than 5%) of compounds of the present disclosure having alternate stereochemistry.

The term “alkyl” as used herein means straight and/or branched chain, saturated alkyl groups containing from one to n carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, wherein n is the maximum number of carbon atoms in the group.

The term “aryl” as used herein refers to a cyclic or polycyclic aromatic ring. For example, the aryl group can be phenyl or napthyl.

The expression “aromatic heterocycle” as used herein refers to an aromatic cyclic or fused polycyclic ring system having at least one heteroatom selected from the group consisting of N, O, S and P. Non-limitative examples include heteroaryl groups are furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

The expression “non-aromatic heterocycle” includes non-aromatic rings or ring systems that contain at least one ring having at least having at least one heteroatom selected from the group consisting of N, O, S and P. This term includes, in a non-limitative manner all of the fully saturated and partially unsaturated derivatives of the above mentioned aromatic heterocycles groups. Examples of non-aromatic heterocycle groups include, in a non-limitative manner, pyrrolidinyl, tetrahydrofuranyl, morpholinyl, thiomorpholinyl, piperidinyl, piperazinyl, thiazolidinyl, isothiazolidinyl, and imidazolidinyl.

The term “suitable”, as in for example, “suitable counter anion” or “suitable reaction conditions” means that the selection of the particular group or conditions would depend on the specific synthetic manipulation to be performed and the identity of the molecule but the selection would be well within the skill of a person trained in the art. All process steps described herein are to be conducted under conditions suitable to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The expression “pharmaceutically acceptable” means compatible with the treatment of subjects such as animals or humans.

The expression “pharmaceutically acceptable salt” means an acid addition salt or basic addition salt which is suitable for or compatible with the treatment of subjects such as animals or humans.

The expression “pharmaceutically acceptable acid addition salt” as used herein means any non-toxic organic or inorganic salt of any compound of the present disclosure, or any of its intermediates. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluenesulfonic and methanesulfonic acids. Either the mono or di-acid salts can be formed, and such salts may exist in either a hydrated, solvated or substantially anhydrous form. In general, the acid addition salts of the compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the present disclosure, for laboratory use, or for subsequent conversion to a pharmaceutically acceptable acid addition salt.

The term “pharmaceutically acceptable basic addition salt” as used herein means any non-toxic organic or inorganic base addition salt of any acid compound of the disclosure, or any of its intermediates. Acidic compounds of the disclosure that may form a basic addition salt include, for example, where CO₂H is a functional group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds of the disclosure, for laboratory use, or for subsequent conversion to a pharmaceutically acceptable acid addition salt.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

Compounds of the present disclosure include prodrugs. In general, such prodrugs will be functional derivatives of these compounds which are readily convertible in vivo into the compound from which it is notionally derived. Prodrugs of the compounds of the present disclosure may be conventional esters formed with available hydroxy, or amino group. For example, an available OH or nitrogen in a compound of the present disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the present disclosure are those in which one or more of the hydroxy groups in the compounds is masked as groups which can be converted to hydroxy groups in vivo. Conventional procedures for the selection and preparation of suitable prodrugs are described, for example, in “Design of Prodrugs” ed. H. Bundgaard, Elsevier, 1985.

The term “cancer” as used herein means a primary or a secondary cancer and includes a non-metastatic cancer and/or a metastatic cancer. Reference to cancer includes reference to cancer cells. For example, the cancer is cervical cancer, breast cancer, ovarian cancer, brain cancer, melanoma, colorectal cancer, glioblastoma, liver cancer, lung cancer, prostate cancer, head cancer, gastric cancer, kidney cancer, endometrial cancer, testis cancer, urothelial cancer, acute lymphoblastic leukemia, acute myeloid leukemia, Hodgkin lymphoma, neuroblastoma, non-Hodgkin lymphoma, soft tissue cancer, bone sarcoma, thyroid cancer, transitional cell bladder cancer, Wilm's tumour, glioma, pancreatic cancer or spleen cancer. For example, the cancer includes any cancer with centrosome aberrations in the cancer cell.

The term “cancer cell” as used herein refers to in vitro cancer cells but also to in vivo cancer cells, e.g. cancer cells present in a subject such as a mammal or a human. For example, in vitro cancer cells may include (human breast cancer) cells, (e.g. BT-549 and MCF-7 cells) mouse neuroblastoma cells (e.g. N1E-115 cells), human non-small cell lung cancer cells (e.g. A549 and H1299 cells), colorectal cancer cells (e.g. HCT116 cells) or human cervical cancer cells (e.g. HeLa).

The term “anti-cancer agent” as used herein means an agent capable of producing a therapeutic effect by inhibiting, suppressing or reducing a cancer (e.g., as determined by clinical symptoms or the amount of cancerous cells) in a subject as compared to a control. Examples of anti-cancer agents include for example non-tubulin-targeting drugs such as doxorubicin, and tubulin-targeting drugs such as taxanes (e.g. paclitaxel), vinca alkaloids (e.g. vinblastine).

The term “anti-mitotic agent” as used herein means an agent that can be used for blocking cancer cell proliferation. For example, the anti-mitotic agent can be an agent that causes microtubule depolymerization such as for example nocodazole or colchicine.

The term “mixture” as used herein, means a composition comprising two or more compounds. In an embodiment a mixture is a mixture of two or more distinct compounds, for example a mixture comprising a compound herein disclosed and an anti-cancer agent such as a taxane for example. In a further embodiment, when a compound is referred to as a “mixture”, it may comprise two or more “forms” of the compounds, such as, salts, solvates, prodrugs or, where applicable, stereoisomers of the compound in any ratio. A person of skill in the art would understand that a compound in a mixture can also exist as a mixture of forms. For example, a compound may exist as a hydrate of a salt or as a hydrate of a salt of a prodrug of the compound. All forms of the compounds disclosed herein are within the scope of the present application.

The term “subject” as used herein includes all members of the animal kingdom including a mammal. In an embodiment, the mammal is a mouse. In another embodiment, the mammal is a human.

The terms “suitable” and “appropriate” refer to the selection of particular groups or conditions that would depend for example on the specific synthetic manipulation to be performed and the identity of the compound, however the selection remains well within the skill of a person trained in the art. All method steps described herein are to be conducted under conditions suitable to provide the product described. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The expression an “effective amount” of a compound or composition of the present disclosure is a quantity sufficient to, when administered to the subject, including a mammal, for example a human, effect beneficial or desired results, including clinical results, and, as such, an “effective amount” depends upon the context in which it is being applied. For example, in the context of treating cancer, for example, it is an amount of the compound or composition, alone or in combination with an anti-cancer agent and/or an anti-mitotic agent, sufficient to achieve treatment of the cancer as compared to a response in the absence of administration of the compound or composition, alone or in combination with an anti-cancer agent and/or an anti-mitotic agent. The amount of a given compound or composition of the present disclosure that corresponds to an effective amount will vary depending upon various factors, such as for example the given drug or compound, the pharmaceutical formulation, the route of administration, the type of disease or disorder, the identity of the subject or host being treated, and the like, but can nevertheless be routinely determined by one skilled in the art. Also, as used herein, an “effective amount” of a compound of the present disclosure is an amount which inhibits, suppresses or reduces a cancer (e.g., as determined by clinical symptoms or the amount of cancerous cells) in a subject as compared to a control. As further used herein, the “effective amount” is an amount that is sufficient to induce mitotic arrest, disrupt centrosome integrity, prevent and/or reduce centrosome clustering, decluster centrosomes and/or alter microtubule dynamics including microtubule depolymerization in a cancer cell.

As used herein, “treatment” or “treating” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” or “treating” can also mean prolonging survival as compared to expected survival if not receiving treatment. “Palliating” a disease or disorder, means that the extent and/or undesirable clinical manifestations of a disorder or a disease state are lessened and/or time course of the progression is slowed or lengthened, as compared to not treating the disorder.

The term “administered” as used herein means administration of a therapeutically effective dose of a composition of the application to a cell either in cell culture or in a patient.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In compositions comprising an “additional” or “second” component, the second component as used herein is chemically different from the other components or first component. A “third” component is different from the other, first, and second components, and further enumerated or “additional” components are similarly different.

The definitions and embodiments described in particular sections are intended to be applicable to other embodiments herein described for which they are suitable as would be understood by a person skilled in the art.

### II. Methods and Compounds

In accordance with a first aspect, there is provided a method for targeting tubulin, ch-TOG, Aurora A kinase, TPX2, Cdk5rap2 and/or ASPM expressed in a cancer cell and selectively inhibiting growth therein, comprising exposing said cancer cell to a compound of Formula I:

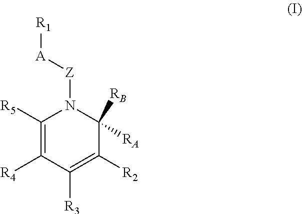

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} said C₆-C₁₂ aryl and said three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, CF₃, CF₂H, CFH₂, F, Cl, Br and I,
   or a pharmaceutically acceptable salt, solvate or prodrug thereof.

For example, targeting tubulin, ch-TOG, Aurora A kinase, TPX2, Cdk5rap2 and/or ASPM induces mitotic arrest in said cancer cell.

For example, targeting tubulin, ch-TOG, Aurora A kinase, TPX2, Cdk5rap2 and/or ASPM is effective for disrupting centrosome integrity, preventing and/or reducing centrosome clustering, declustering centrosomes, regulating centrosome clustering and/or altering microtubule dynamics including microtubule depolymerization in a cancer cell.

According to an aspect, there is provided herein a method for targeting tubulin, ch-TOG, Aurora A kinase, TPX2, Cdk5rap2 and/or ASPM expressed in a cancer cell and selectively inhibiting growth therein, comprising exposing said cancer cell to a combination of a compound of Formula I and an anti-cancer agent and/or an anti-mitotic agent.

According to an aspect, there is provided herein a method for targeting tubulin, ch-TOG, Aurora A kinase, TPX2, Cdk5rap2 and/or ASPM expressed in a cancer cell and inhibiting growth therein, comprising exposing said cancer cell to a synergistic combination of a compound of Formula I and an anti-cancer agent and/or an anti-mitotic agent, wherein said combination more than additively inhibits growth of said cancer cell.

For example, the cancer cell expresses ch-TOG.

For example, the cancer cell expresses Aurora A kinase.

For example, the cancer cell expresses TPX2.

For example, the cancer cell expresses tubulin.

For example, the cancer cell expresses Cdk5rap2.

For example, the cancer cell expresses ASPM.

For example, targeting tubulin, ch-TOG, Aurora A kinase, TPX2, Cdk5rap2 and/or ASPM is effective for treating a cancer in a subject.

According to an aspect, there is provided herein a method for selectively inhibiting growth in a cancer cell, comprising exposing the cancer cell to a compound of Formula I:

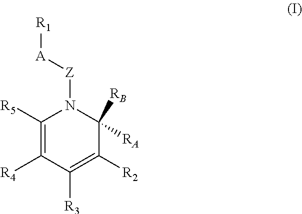

wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} the C₆-C₁₂ aryl and the three- to seven-membered aromatic heterocycle being each independently unsubstituted or substituted with at least one substituent chosen from C₁-C₆ alkyl, C₁-C₆ alkoxy, C₆-C₁₂ aryl, the three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂ CF₃, CF₂H, CFH₂, F, Cl, Br and I, OH, CHO,
or a pharmaceutically acceptable salt, solvate or prodrug thereof.

For example, inhibiting growth comprises inducing mitotic arrest in the cancer cell.

According to another aspect, there is provided herein a method for disrupting centrosome integrity, preventing and/or reducing centrosome clustering, declustering centrosomes, regulating centrosome clustering and/or altering microtubule dynamics including microtubule depolymerization in a cancer cell, comprising exposing the cancer cell to a compound of Formula I.

According to yet another aspect, there is provided herein a method for selectively inhibiting growth in a cancer cell, comprising exposing the cancer cell to a combination of a compound of Formula I and an anti-cancer agent and/or an anti-mitotic agent.

According to another aspect, there is provided herein a method for inhibiting growth in a cancer cell, comprising exposing the cancer cell to a synergistic combination of a compound of Formula I and an anti-cancer agent and/or an anti-mitotic agent, wherein the combination more than additively inhibits growth of the cancer cell.

According to another aspect, there is provided herein a method for increasing selectivity of an anti-cancer agent and/or an anti-mitotic agent to a cancer cell, comprising exposing the cancer cell with a compound of Formula I and the anti-cancer agent and/or the anti-mitotic agent.

For example, the cancer cells are contacted with a thienoisoquinoline compound herein disclosed at a concentration in the nanomolar range. For example, the method comprises exposing the cancer cell to the compound having a concentration of about 1 nM, about 5 nM, about 100 nM, about 150 nM, about 200 nM, about 250 nM, about 300 nM, about 350 nM, about 400 nM, about 450 nM, about 500 nM, about 550 nM, about 600 nM, about 1000 nM, about 5000 nM or about 10000 nM.

For example, the method comprises exposing the cancer cell to the compound for at least 1 minute, 2 minutes, 3 minutes, 4 minutes, 5 minutes, 10 minutes, 15 minutes, 20 minutes, 30 minutes, at least 45 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 24 hours, at least 30 hours, at least 36 hours, at least 42 hours, at least 48 hours, at least 60 hours, at least 72 hours or at least 80 hours. For example, the cancer cells can also be contacted with a thienoisoquinoline compound herein disclosed for a population doubling time. The term “population doubling time” is to be understood as the period of time required to double the cell population.

For example, the cancer cell is a cancer cell with aberrant centrosomes.

For example, the cancer cell is a breast cancer cell, a cervical cancer cell, a lung cancer cell, a pancreatic cancer cell, a colorectal cancer cell, a neuroblastoma cancer cell or an ovarian cancer cell.

For example, the cancer cell is a mammal cancer cell.

For example, the mammal cancer cell is a human cancer cell.

For example, the method is carried out in vitro.

For example, the method is carried out in vivo.

For example, the cancer cell is present in a subject.

For example, the subject is a mammal.

For example, the mammal is a human.

According to another aspect, there is provided herein a method of treating a cancer in a subject, comprising administering to the subject an effective amount of a compound of Formula I.

According to another aspect, there is provided herein a method of treating a cancer in a subject, comprising administering to the subject an effective amount of a combination of a compound of Formula I and an anti-cancer agent and/or an anti-mitotic agent.

For example, the cancer is a cancer with aberrant centrosomes.

For example, the cancer is breast cancer, cervical cancer, lung cancer, pancreatic cancer, colorectal cancer, neuroblastoma cancer or ovarian cancer.

For example, the subject is a mammal.

For example, the mammal is a human.

For example, the compound for Formula I and anti-cancer agent and/or anti-mitotic agent are administered sequentially or concomitantly.

For example, the anti-cancer agent is a taxane, a vinca alkaloid or a colchicine-site binder.

For example, the anti-cancer agent is a non-mitotic anti-cancer agent.

For example, the anti-mitotic agent is nocodazole.

For example, the cancer cells are further contacted with nocodazole at a concentration of about 5 nM, about 10 nM, about 15 nM, about 20 nM, about 25 nM, about 30 nM, about 33 nM, about 35 nM, about 40 nM, about 45 nM, about 50 nM, about 55 nM, about 60 nM, about 66 nM, about 70 nM, about 75 nM, about 100 nM, about 125 nM or about 135 nM.

For example, the taxane is paclitaxel, cabazitaxel, or docetaxel.

For example, the cancer cells are further contacted with a taxane at a concentration of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, about 19 nM, about 20 nM, about 25 nM, about 30 nM, about 35 nM, about 40 nM, about 45 nM, about 50 nM, about 60 nM, about 70 nM, about 80 nM, about 90 nM or about 100 nM.

For example, the vinca alkaloid is vinblastine, vincristine, vindesine, and vinorelbine.

For example, the colchicine-site binder is colchicine, a combrestatin or podophyllotoxin.

For example, the non-mitotic anti-cancer agent is doxorubicin, an anthracycline, an alkylating drug or an antimetabolite.

For example, the compound of Formula I is a compound of Formula IA:

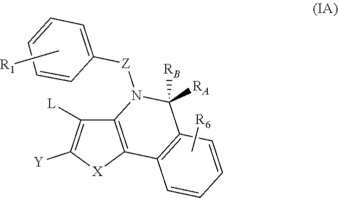

L is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
X is S, O, NH, CH—CH, CH—N, N—CH or NR₇;
Y is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₆ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, F, C₁-C₆ sulfonylakyl, C₁-C₆ aminoalkyl, C₁-C₆ alkylamino, CN, CF₃, CF₂H, CFH₂, Cl, Br, I, a C₆-C₁₂ aryl or a three- to seven-membered aromatic heterocycle; and
R₇ is C₁-C₆ alkyl, C₁-C₆ haloalkyl, C₁-C₆ sulfonylakyl, C₁-C₆ alkylamino, CF₃, CF₂H, CFH₂, a C₆-C₁₂ aryl or a three- to seven-membered aromatic heterocycle,
L, R_{A}, R_{B}, R₁, R₆, R₇, the C₆-C₁₂ aryl and the three- to seven-membered aromatic heterocycle being each independently unsubstituted or, when possible, substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, CN, C₁-C₆ aminoalkyl, C₁-C₆ alkylamino, CF₃, CF₂H, CFH₂, F, Cl, Br and I,
or a pharmaceutically acceptable salt, solvate or prodrug thereof.

For example, the compound of Formula I is a compound of Formula IA:

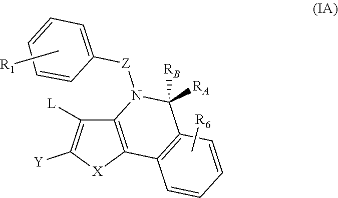

wherein
L is H, C₁-C₃ alkyl, C₁-C₃ alkoxy, C₁-C₃ hydroxyalkyl, C₁-C₃ alkythio, C₁-C₃ thioalkyl, C₁-C₃ sulfonylakyl, C₁-C₃ aminoalkyl, C₁-C₃ alkylamino, ON, CF₃, CF₂H, CFH₂, F, Cl, Br, I;
X is S, O, NR₇ or NH;
Y is F, Cl, Br, I, H, CH₃, CF₃, CHF₂, CF₂H or CN;
Z is SO₂, CO or CH₂;
R_{A} and R_{B} are each independently H, C₁-C₃ alkyl, C₁-C₃ hydroxyalkyl, C₁-C₃ sulfonylakyl, C₁-C₃ alkylamino, CF₃, CF₂H, CFH₂, F;
R₁ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I;
R₆ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I; and
R₇ is C₁-C₆ alkyl, C₁-C₆ haloalkyl, C₁-C₆ sulfonylakyl, C₁-C₆ alkylamino, CF₃, CF₂H, CFH₂, a C₆-C₁₂ aryl or a three- to seven-membered aromatic heterocycle,
L, R_{A}, R_{B}, R₁, R₆, the C₆-C₁₂ aryl and the three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ haloalkyl, F, Cl, Br and I,
or a pharmaceutically acceptable salt, solvate or prodrug thereof.

For example, the compound of Formula I is a compound of Formula IA:

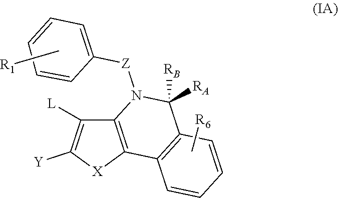

wherein
L is H;
X is S;
Z is SO₂, CO or CH₂;
Y is F, Cl, Br or I;
R_{A} is H;
R_{B} is H;
R₁ is in para position with respect to Z and is chosen from C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br and I; and
R₆ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I,
R₁, R₆, the C₆-C₁₂ aryl and the three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ haloalkyl, F, Cl, Br and I,
   or a pharmaceutically acceptable salt, solvate or prodrug thereof.

For example,
L is H;
Z is SO₂;
Y is H, F, Cl, Br, I or C₆-C₁₂ aryl;
R_{A} is H;
R_{B} is H;
R₁ is H, C₁-C₆ alkyl, C₁-C₃ haloalkyl or C₁-C₃ alkoxy; and
R₆ is H.

For example,
L is H;
Z is SO₂;
Y is H, F, Cl, Br, I or phenyl;
R_{A} is H;
R_{B} is H;
R₁ is H, C₁-C₃ alkyl, CF₃ or methoxy; and
R₆ is H.

For example, R₁, R_{A}, R_{B} said C₆-C₁₂ aryl and said three- to seven-membered aromatic heterocycle can be each independently unsubstituted or substituted with 2 or 3 or 4 substituents chosen from C₁-C₆ alkyl, C₁-C₆ alkoxy, C₆-C₁₂ aryl, three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂, CF₃, CF₂H, CFH₂, F, Cl, Br and I, OH, CHO, COOH and COOR_{C}, wherein R_{C} is a C₁-C₆ alkyl.

For example, the compound is chosen from

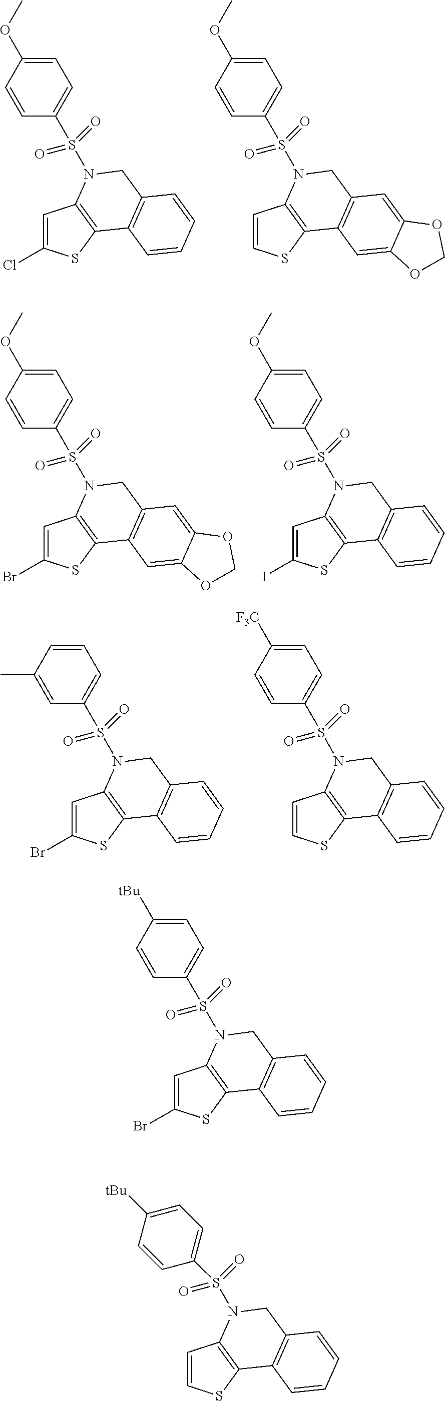

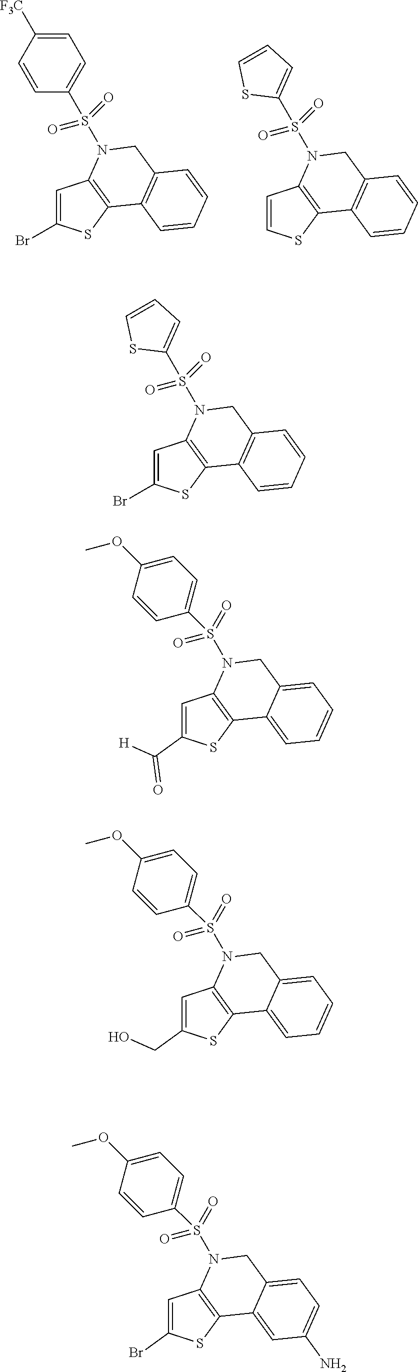

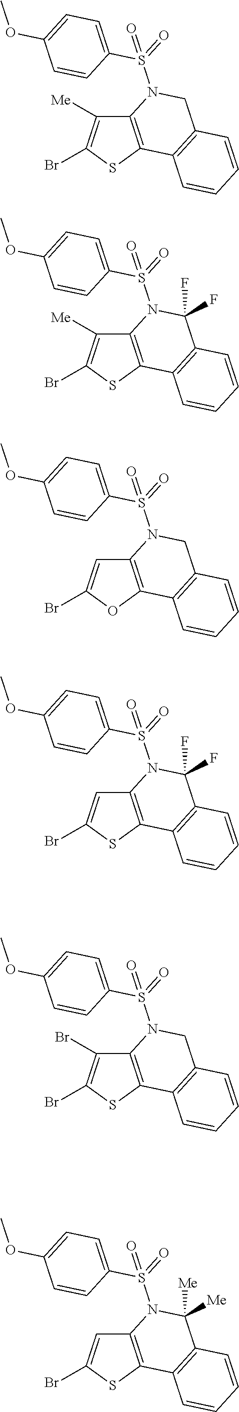

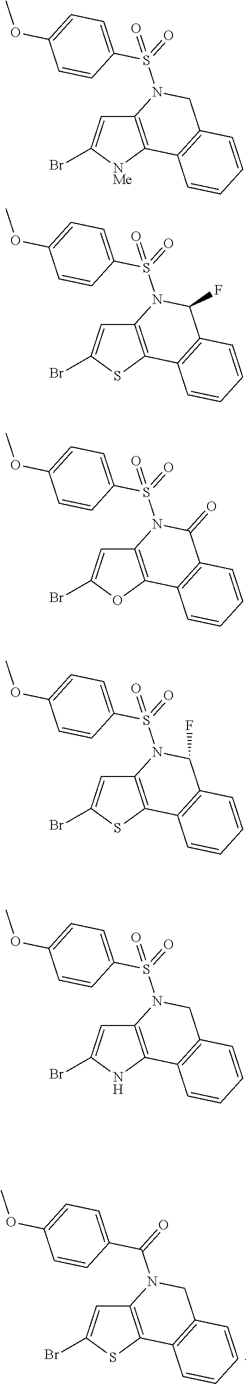 [SMILES: COc1ccc(S(=O)(=O)N2Cc3ccccc3-c3c2cc(Br)n3C)cc1 ; COc1ccc(S(=O)(=O)N2c3cc(Br)sc3-c3ccccc3[C@@H]2F)cc1 ; COc1ccc(S(=O)(=O)n2c(=O)c3ccccc3c3oc(Br)cc32)cc1 ; COc1ccc(S(=O)(=O)N2c3cc(Br)sc3-c3ccccc3[C@H]2F)cc1 ; COc1ccc(S(=O)(=O)N2Cc3ccccc3-c3[nH]c(Br)cc32)cc1 ; COc1ccc(C(=O)N2Cc3ccccc3-c3sc(Br)cc32)cc1]

For example, the compound is

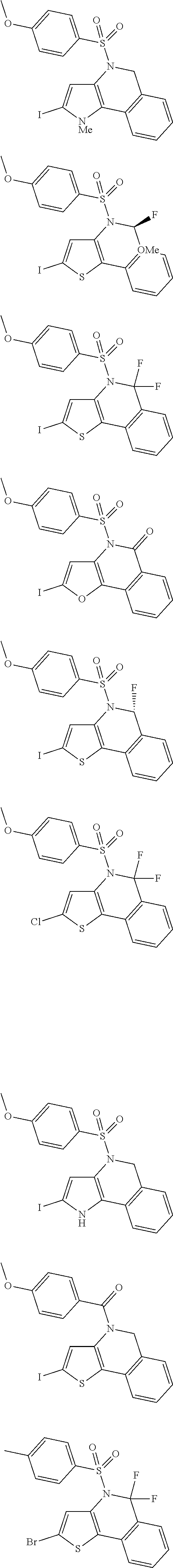

For example, the compound is

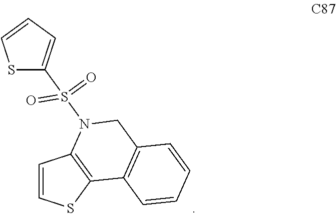 [SMILES: O=S(=O)(c1cccs1)N1Cc2ccccc2-c2sccc21]

For example, the compound is

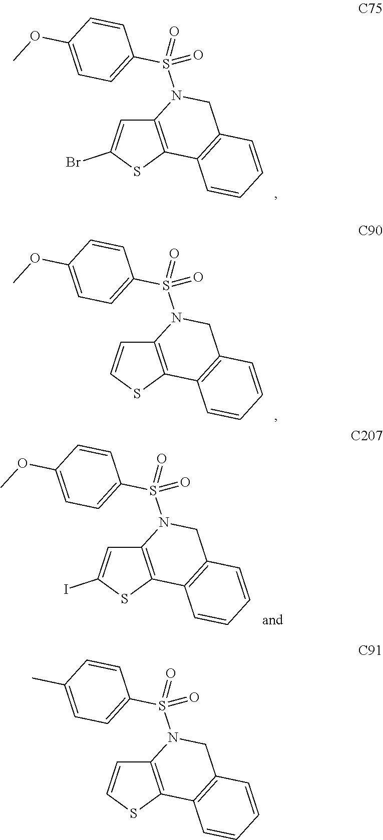 [SMILES: COc1ccc(S(=O)(=O)N2Cc3ccccc3-c3sc(Br)cc32)cc1 ; COc1ccc(S(=O)(=O)N2Cc3ccccc3-c3sccc32)cc1 ; COc1ccc(S(=O)(=O)N2Cc3ccccc3-c3sc(I)cc32)cc1 ; Cc1ccc(S(=O)(=O)N2Cc3ccccc3-c3sccc32)cc1]

For example, the compound is

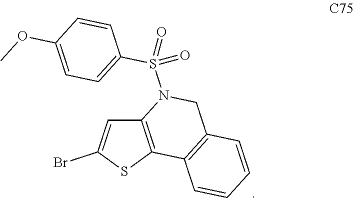 [SMILES: COc1ccc(S(=O)(=O)N2Cc3ccccc3-c3sc(Br)cc32)cc1]

According to a further aspect, there is provided herein a use of a compound of Formula I for targeting tubulin, ch-TOG, Aurora A kinase, TPX2, Cdk5rap2 and/or ASPM expressed in a cancer cell and selectively inhibiting growth therein.

For example, targeting tubulin, ch-TOG, Aurora A kinase, TPX2, Cdk5rap2 and/or ASPM is effective for disrupting centrosome integrity, preventing and/or reducing centrosome clustering, declustering centrosomes, regulating centrosome clustering and/or altering microtubule dynamics including microtubule depolymerization in a cancer cell.

Yet another aspect provided herein relates to a use of a combination of a compound of Formula I and an anti-cancer agent and/or an anti-mitotic agent for targeting tubulin, ch-TOG, Aurora A kinase, TPX2, Cdk5rap2 and/or ASPM expressed in a cancer cell and selectively inhibiting growth therein.

For example, targeting tubulin, ch-TOG, Aurora A kinase, TPX2, Cdk5rap2 and/or ASPM induces mitotic arrest in said cancer cell.

For example, targeting tubulin, ch-TOG, Aurora A kinase, TPX2, Cdk5rap2 and/or ASPM is effective for treating a cancer in a subject.

For example, the compound of Formula I is used in combination with an anti-cancer agent and/or an anti-mitotic agent.

For example, the use is effective for increasing selectivity of said anti-cancer agent and/or said anti-mitotic agent to a cancer cell.

For example, the cancer cell expresses ch-TOG.

For example, the cancer cell expresses Aurora A kinase.

For example, the cancer cell expresses TPX2.

For example, the cancer cell expresses tubulin.

For example, the cancer cell expresses Cdk5rap2.

For example, the cancer cell expresses ASPM.

According to a further aspect, there is provided herein a use of a compound of Formula I for selectively inhibiting growth in a cancer cell.

According to another aspect, there is provided herein a use of a compound of Formula I for disrupting centrosome integrity, preventing and/or reducing centrosome clustering, declustering centrosomes, regulating centrosome clustering and/or altering microtubule dynamics including microtubule depolymerization in a cancer cell.

According to yet another aspect, there is provided herein a use of a combination of a compound of Formula I and an anti-cancer agent and/or an anti-mitotic agent in a cancer cell.

For example, inhibiting growth in the cancer cell is selective.

For example, inhibiting growth comprises inducing mitotic arrest in the cancer cell.

According to another aspect, there is provided herein a use of a compound of Formula I for the treatment of cancer in a subject.

According to another aspect, there is provided herein a use of a combination of a Formula I and an anti-cancer agent and/or an anti-mitotic agent for the treatment of cancer in a subject.

According to another aspect, there is provided herein a use of a compound of Formula I in combination with an anti-cancer agent and/or an anti-mitotic agent for increasing selectivity of the anti-cancer agent and/or the anti-mitotic agent to a cancer cell.

For example, the compound herein disclosed and/or the combination comprising the compound and an anti-cancer agent and/or an anti-mitotic agent herein disclosed are comprised in a composition that comprises an injectable dosage form. For example, the composition is administered by intratumoral injection.

For example, the compound herein disclosed and/or the combination comprising the compound and an anti-cancer agent and/or an anti-mitotic agent herein disclosed are comprised in a composition administered by parenteral, intravenous, subcutaneous, intramuscular, intracranial, intraspinal, intracisternal, intraperitoneal, intranasal, aerosol or oral administration.

According to a further aspect, there is provided herein a compound of Formula IA:

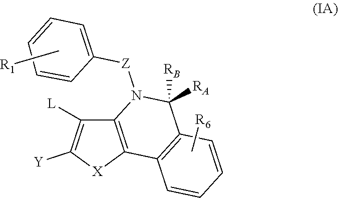

wherein
L is H, C₁-C₃ alkyl, C₁-C₃ alkoxy, C₁-C₃ thioalkyl, C₁-C₃ haloalkyl, CN, CF₃, CF₂H, CFH₂, F, Cl, Br or I;
X is S, O, NR₇ or NH;
Y is F, Cl, Br, I, H, CH₃, CF₃, CHF₂, CF₂H or CN;
Z is SO₂, CO or CH₂;
R_{A} and R_{B} are each independently H, Me, Et, CF₃, CF₂H, CFH₂, F or Cl;
R₁ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I;
R₆ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I; and
R₇ is C₁-C₆ alkyl, C₁-C₆ haloalkyl, C₁-C₆ sulfonylakyl, C₁-C₆ alkylamino, CF₃, CF₂H, CFH₂, a C₆-C₁₂ aryl or a three- to seven-membered aromatic heterocycle,
L, R_{A}, R_{B}, R₁, R₆, the C₆-C₁₂ aryl and the three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ haloalkyl, F, Cl, Br and I,
or a pharmaceutically acceptable salt, solvate or prodrug thereof.

For example,
L is H;
X is S;
Z is SO₂, CO or CH₂;
Y is F, Cl, Br or I;
R_{A} is H;
R_{B} is H;
R₁ is in para position with respect to Z and is chosen from C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br and I; and
R₆ is C₁-C₃ alkyl, C₁-C₃ alkoxy, C₁-C₃ hydroxyalkyl, C₁-C₃ sulfonylakyl, C₁-C₃ aminoalkyl, C₁-C₃ alkylamino, CN, CF₃, CF₂H, CFH₂, F, Cl, Br or I,
R₁, R₆, the C₆-C₁₂ aryl and the three- to seven-membered aromatic heterocycle being each independently unsubstituted or substituted with at least one substituent chosen from C₁-C₆ alkyl, C₁-C₆ alkoxy, C₁-C₆ hydroxyalkyl, C₁-C₆ alkythio, C₁-C₆ thioalkyl, C₁-C₆ haloalkyl, F, Cl, Br and I,
or a pharmaceutically acceptable salt, solvate or prodrug thereof.

For example,
L is H;
Z is SO₂;
Y is H, F, Cl, Br, I or C₆-C₁₂ aryl;
R_{A} is H;
R_{B} is H;
R₁ is H, C₁-C₆ alkyl, C₁-C₃ haloalkyl or C₁-C₃ alkoxy; and
R₆ is H.

For example,
L is H;
Z is SO₂;
Y is H, F, Cl, Br, I or phenyl;
R_{A} is H;
R_{B} is H;
R₁ is H, C₁-C₃ alkyl, CF₃ or methoxy; and
R₆ is H.

For example, the compound is chosen from

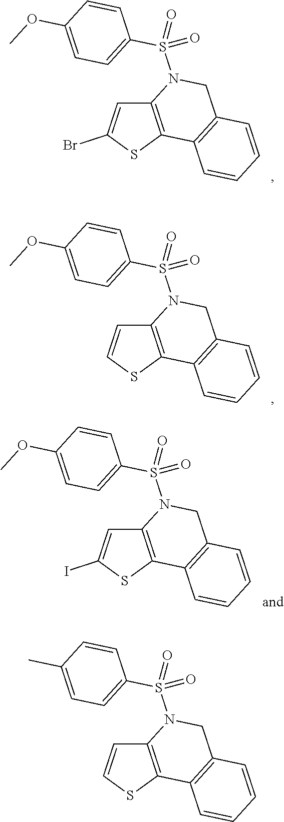 [SMILES: COc1ccc(S(=O)(=O)N2Cc3ccccc3-c3sc(Br)cc32)cc1 ; COc1ccc(S(=O)(=O)N2Cc3ccccc3-c3sccc32)cc1 ; COc1ccc(S(=O)(=O)N2Cc3ccccc3-c3sc(I)cc32)cc1 ; Cc1ccc(S(=O)(=O)N2Cc3ccccc3-c3sccc32)cc1]

For example, the compound is

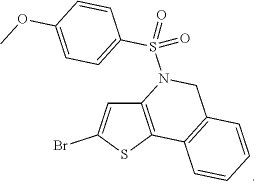 [SMILES: COc1ccc(S(=O)(=O)N2Cc3ccccc3-c3sc(Br)cc32)cc1]

According to a further aspect, there is provided herein a compound chosen from:

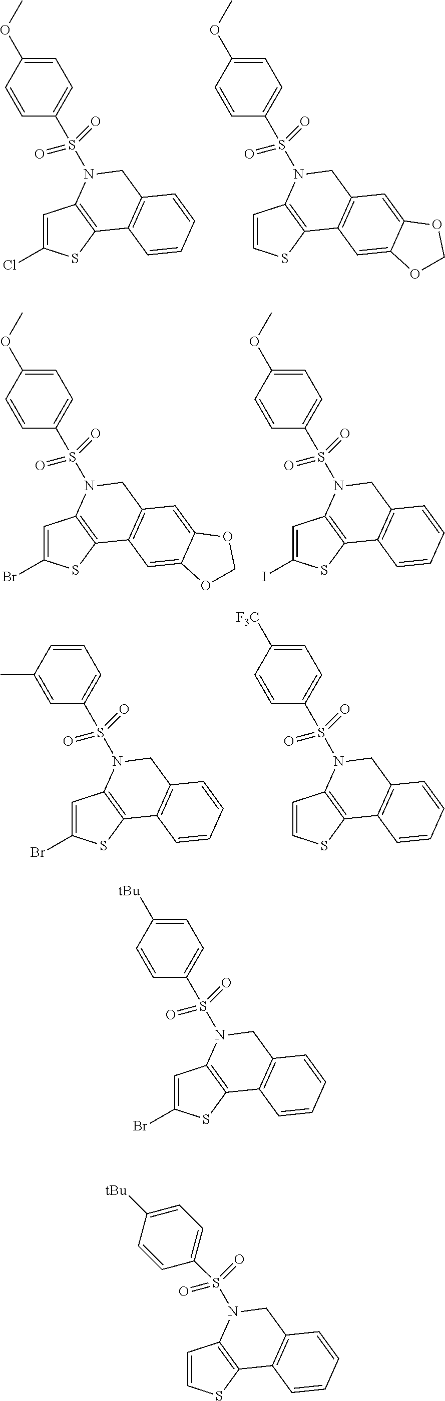

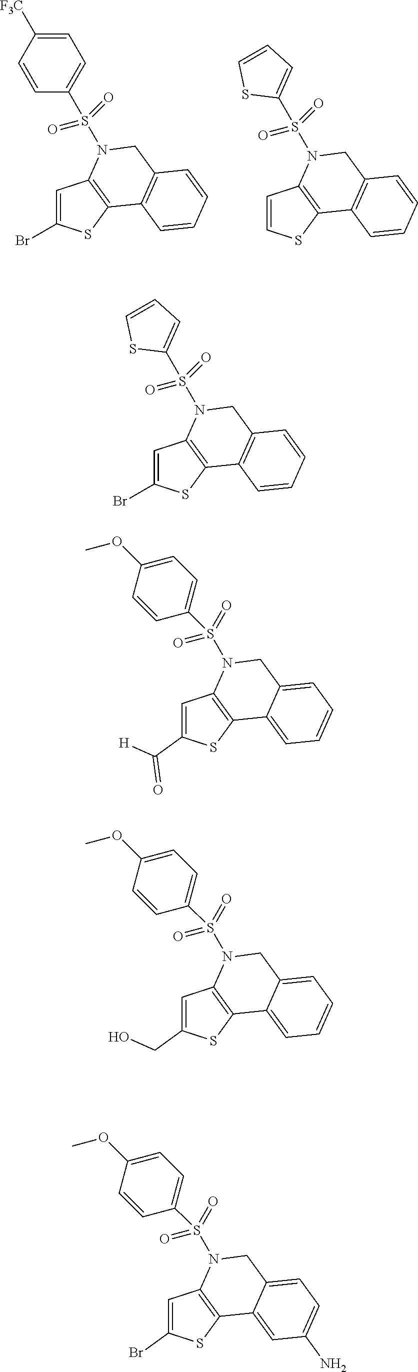

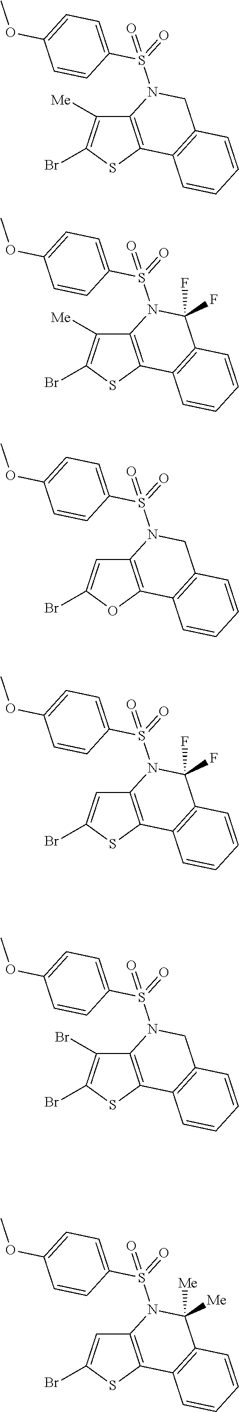

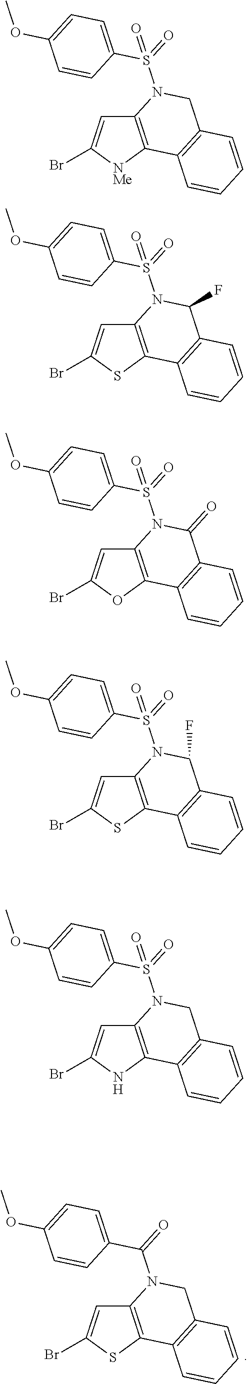 [SMILES: COc1ccc(S(=O)(=O)N2Cc3ccccc3-c3c2cc(Br)n3C)cc1 ; COc1ccc(S(=O)(=O)N2c3cc(Br)sc3-c3ccccc3[C@@H]2F)cc1 ; COc1ccc(S(=O)(=O)n2c(=O)c3ccccc3c3oc(Br)cc32)cc1 ; COc1ccc(S(=O)(=O)N2c3cc(Br)sc3-c3ccccc3[C@H]2F)cc1 ; COc1ccc(S(=O)(=O)N2Cc3ccccc3-c3[nH]c(Br)cc32)cc1 ; COc1ccc(C(=O)N2Cc3ccccc3-c3sc(Br)cc32)cc1]

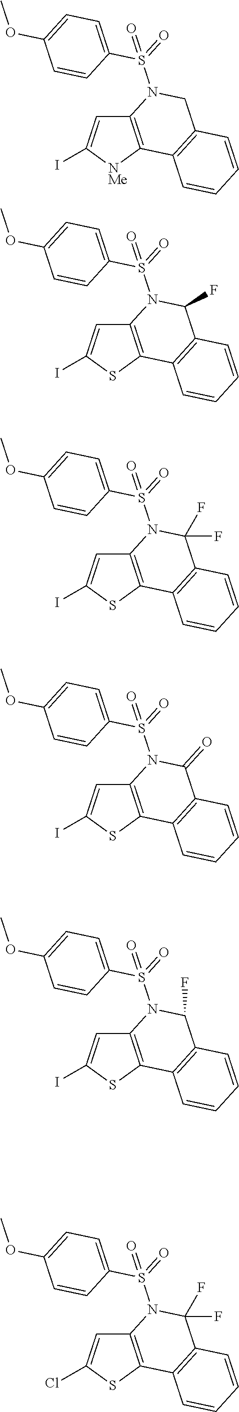 [SMILES: COc1ccc(S(=O)(=O)N2Cc3ccccc3-c3c2cc(I)n3C)cc1 ; COc1ccc(S(=O)(=O)N2c3cc(I)sc3-c3ccccc3[C@@H]2F)cc1 ; COc1ccc(S(=O)(=O)N2c3cc(I)sc3-c3ccccc3C2(F)F)cc1 ; COc1ccc(S(=O)(=O)n2c(=O)c3ccccc3c3sc(I)cc32)cc1 ; COc1ccc(S(=O)(=O)N2c3cc(I)sc3-c3ccccc3[C@H]2F)cc1 ; COc1ccc(S(=O)(=O)N2c3cc(Cl)sc3-c3ccccc3C2(F)F)cc1]

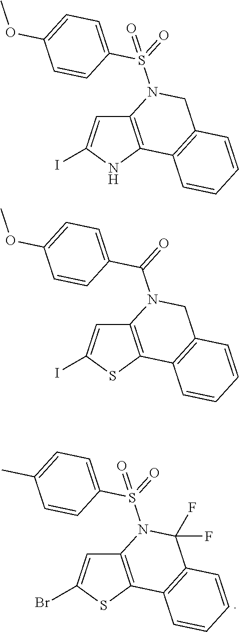 [SMILES: COc1ccc(S(=O)(=O)N2Cc3ccccc3-c3[nH]c(I)cc32)cc1 ; COc1ccc(C(=O)N2Cc3ccccc3-c3sc(I)cc32)cc1 ; Cc1ccc(S(=O)(=O)N2c3cc(Br)sc3-c3ccccc3C2(F)F)cc1]

### EXAMPLES

These examples are not to be construed as limiting the scope of the present disclosure in any way.

### Example 1

A family of high-quality compounds with drug-like properties and potential for medicinal use were identified. A novel synthesis was designed to improve the yield of these compounds in a cost-effective manner and their ability to treat cancers was evaluated. More specifically, a novel synthesis for a high quality, small molecular weight thienoisoquinoline scaffold was designed^{[10,11]}.

To date, 30 variants with different structural modifications have been made. Table 3 below shows IC50 values for a subset of thienoisoquinoline compounds in HeLa (cervical adenocarcinoma), BT549 (breast ductal carcinoma), A549 (lung carcinoma) and MCF-7 (mammary gland carcinoma) cells and HFF1 (fibroblast) and MCF-10A (mammary gland fibrocystic disease) non-cancer cells. Several thienoisoquinoline derivatives, for example C75, C91 and C207, show higher efficacy, i.e. more strongly affect viability, in cancer cells vs. non-cancer cells. In particular, it is shown that C75 causes death in multiple cancer cells with IC₅₀ values in the 100-400 nM range, including breast cancers (MCF7, BT549), lung cancer (A549), colorectal cancer (HCT116) in addition to HeLa cells (cervical cancer).

TABLE 3

| | |
|---|---|
| | C87 |
| 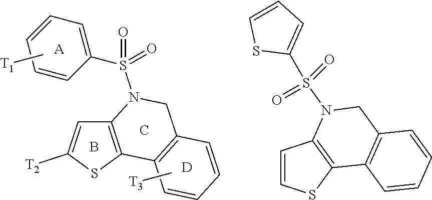 | |

| | | | | IC₅₀ - | | IC₅₀- | | IC₅₀- | | IC₅₀- | | IC₅₀- | | IC₅₀- | |
|---|---|---|---|---|---|---|---|---|---|---|---|---|---|---|---|
| | T₁ | T₂ | T₃ | HeLa | | BT549 | | A549 | | MCF-7 | | HFF-1 | | MCF10A | |
| C39 | 3-Me | H | H | >1 | μM | — | | — | | — | | — | | — | |
| C71 | 4-Me | Ph | H | >1 | μM | — | | — | | — | | — | | — | |
| C74 | 4-Me | Br | H | >1 | μM | — | | — | | — | | — | | — | |
| C75 | 4-OMe | Br | H | 27 | nM | 158 | nM | 109 | nM | 101 | nM | 467 | nM | 309 | nM |
| C87 | — | — | — | 845 | nM | 4198 | nM | 750 | nM | 2463 | nM | >1000 | nM | 539 | nM |

| C90 | 4-OMe | H | H | 177 | nM | — | — | 149 | nM | — | | — |
|---|---|---|---|---|---|---|---|---|---|---|---|---|
| C91 | 4-Me | H | H | 197 | nM | — | — | 1015 | nM | 920 | nM | — |

| C93 | 3-Me | Br | H | >1 | μM | — | | — | — | | — | | — |
|---|---|---|---|---|---|---|---|---|---|---|---|---|---|
| C108 | 4-CF₃ | H | H | >1 | μM | — | | — | — | | — | | — |
| C200 | 4-CF₃ | Br | H | >1 | μM | — | | — | — | | — | | — |
| C201 | 4-tBu | Br | H | >1 | μM | — | | — | — | | — | | — |
| C207 | 4-OMe | I | H | 70 | nM | 178 | nM | — | 86 | nM | 150 | nM | — |

| | | | | | | | | | |
|---|---|---|---|---|---|---|---|---|---|
| C208 | 4-OMe | Cl | H | 204 | nM | — | — | — | — |

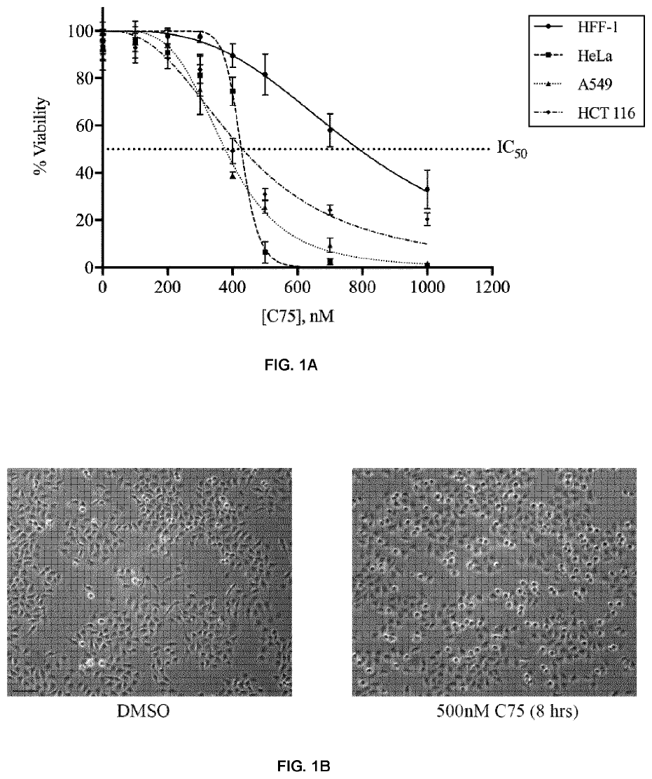

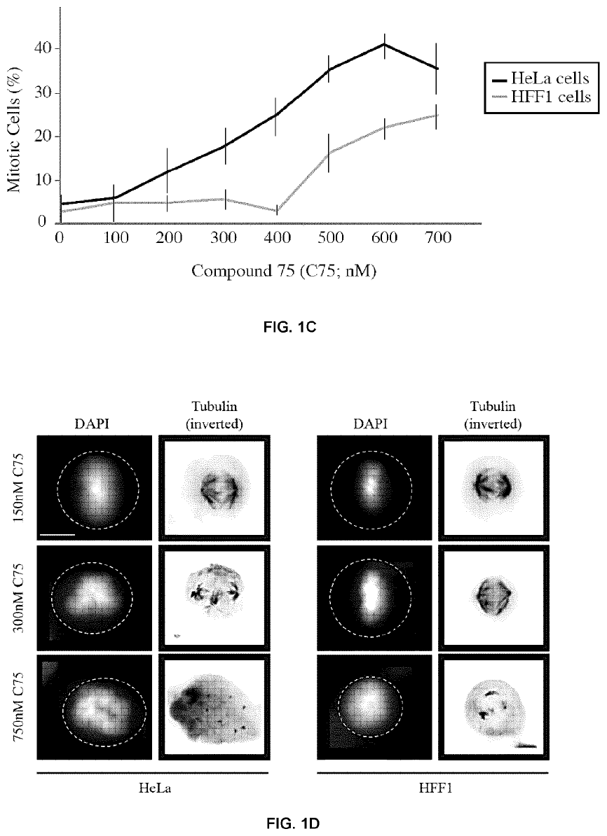

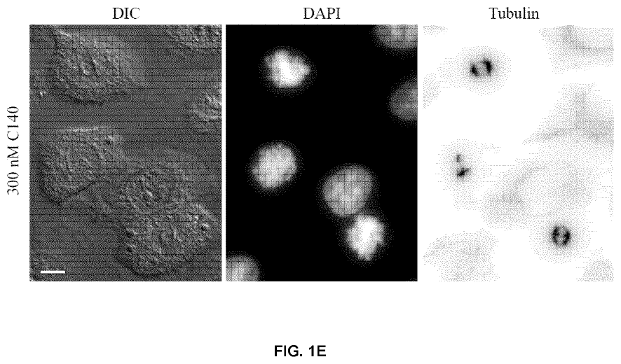

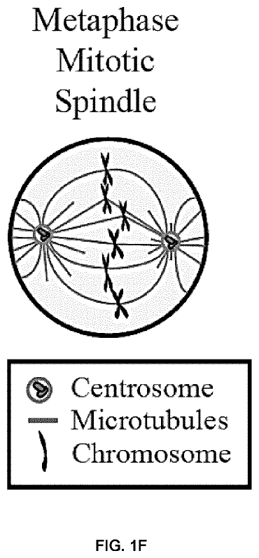

C75 was tested using high-throughput automated analysis, which is more robust compared to prior testing and found to selectively cause toxicity in HeLa, A549 and HCT116 cancer cells compared to HFF-1 non-cancer cells (FIG. 1A and Table 4). C75 was found to cause mitotic arrest in HeLa cells. The images in FIG. 1B) show fields of view of HeLa cells treated with (dimethyl sulfoxide) DMSO or 500 nM of C75. Further testing of C75 revealed it selectively causes mitotic arrest in HeLa cells in comparison to HFF1 (non-cancerous fibroblast) cells at concentrations in the nanomolar range (FIG. 1C, Table 3). While the mitotic spindle was not affected in HeLa cells treated with DMSO or in HFF1 cells treated with DMSO or 300 nM C75, spindle organization was perturbed in HeLa cells treated with 300 nM C75. Centrosomes were fragmented or declustered and microtubules were completely absent in HeLa cells treated with 750 nM C75 (FIG. 1D). Higher concentrations of C75 caused spindle phenotypes in HFF1 cells, but they were quite different in comparison to HeLa cells (FIG. 1D).

TABLE 4

IC₅₀, nM

| Cell Lines | Compound 75 | Compound 87 |
|---|---|---|
| HFF-1 | 789 | >1000 |
| HeLa | 427 | >1000 |
| A549 | 377 | >1000 |
| HCT 116 | 431 | >1000 |

Table 4 shows the calculated IC₅₀ values for C75 and C87 for HFF-1, HeLa, A549 and HCT 116 cell lines.

Another active derivative, C140, has higher efficacy in A549 cells in comparison to C75 (Table 5), and causes spindle phenotypes similar to C75 (FIG. 1E). This shows that multiple derivatives likely bind to the same molecular target.

TABLE 5

| | |
|---|---|
| | (ID) |
| 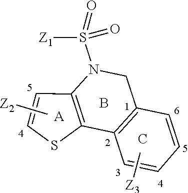 | |

| Compound | Z₁ | Z₂ | Z₃ | cLogP | IC₅₀ (nM) |
|---|---|---|---|---|---|
| C39 | 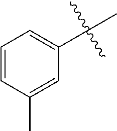 [SMILES: *c1cccc(C)c1] | H | H | 5.057 | >1000 |
| C71 | 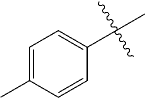 [SMILES: *c1ccc(C)cc1] | 5-C₆H₆ | H | 7.155 | >1000 |
| C72 | 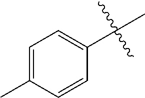 [SMILES: *c1ccc(C)cc1] | 5-Anisole | H | 7.102 | >1000 |
| C74 | 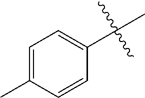 [SMILES: *c1ccc(C)cc1] | 5-Bromo | H | 6.024 | >1000 |
| C75 | 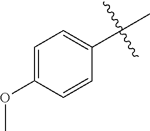 [SMILES: *c1ccc(OC)cc1] | 5-Bromo | H | 5.694 | 430.9 |
| C80 | 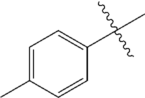 [SMILES: *c1ccc(C)cc1] | 5-Naphthalene | H | 8.329 | >1000 |
| C87 | 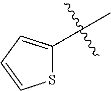 [SMILES: *c1cccs1] | H | H | 4.279 | >1000 |
| C90 | 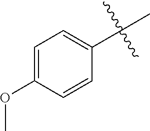 [SMILES: *c1ccc(OC)cc1] | H | H | 4.727 | 780.7 |
| C91 | 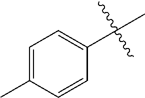 [SMILES: *c1ccc(C)cc1] | H | H | 5.057 | >1000 |
| C108 | 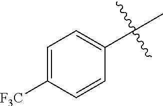 [SMILES: *c1ccc(C(F)(F)F)cc1] | H | H | 5.441 | >1000 |
| C109 | 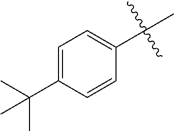 [SMILES: *c1ccc(C(C)(C)C)cc1] | H | H | 6.384 | >1000 |
| C127 | 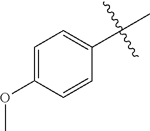 [SMILES: *c1ccc(OC)cc1] | 4-CH₃ | H | 5.226 | 635.2 |
| C128 | 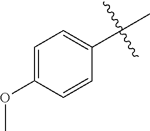 [SMILES: *c1ccc(OC)cc1] | 4-CH₃, 5-Chloro | H | 6.031 | 549.4 |
| C130 | 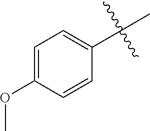 [SMILES: *c1ccc(OC)cc1] | H | 3-NO₂ | 4.501 | 439.2 |
| C131 | 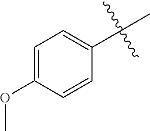 [SMILES: *c1ccc(OC)cc1] | 5-Bromo | 3-NO₂ | 5.447 | 801.1 |
| C132 | 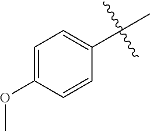 [SMILES: *c1ccc(OC)cc1] | H | 3-NH₂ | 3.540 | >1000 |
| C133 | 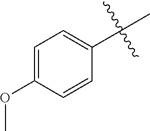 [SMILES: *c1ccc(OC)cc1] | 5-Bromo | 3-NH₂, 6-Bromo | 5.614 | >1000 |
| C138 | 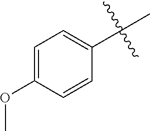 [SMILES: *c1ccc(OC)cc1] | 4-CH₃, 5-CHO | H | 4.967 | 416.4 |
| C139 | 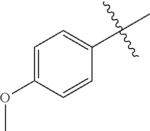 [SMILES: *c1ccc(OC)cc1] | 4-CH₃, 5-Bromo | H | 6.193 | >1000 |
| C140 | 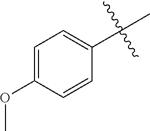 [SMILES: *c1ccc(OC)cc1] | 5-CHO | H | 4.469 | 260.1 |
| C141 | 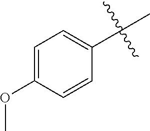 [SMILES: *c1ccc(OC)cc1] | 5-COOH | H | 4.670 | >1000 |
| C142 | 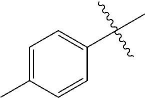 [SMILES: *c1ccc(C)cc1] | 5-CHO | H | 4.799 | 456.3 |
| C143 | 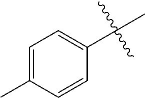 [SMILES: *c1ccc(C)cc1] | 5-COOH | H | 5.000 | >1000 |
| C200 | 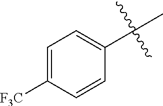 [SMILES: *c1ccc(C(F)(F)F)cc1] | 5-Bromo | H | 6.408 | >1000 |
| C201 | 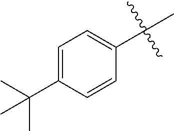 [SMILES: *c1ccc(C(C)(C)C)cc1] | 5-Bromo | H | 7.351 | >1000 |
| C207 | 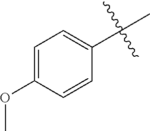 [SMILES: *c1ccc(OC)cc1] | 5-Iodo | H | 5.954 | 433.3 |
| C208 | 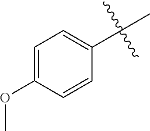 [SMILES: *c1ccc(OC)cc1] | 5-Chloro | H | 5.544 | 434.3 |
| C300 | 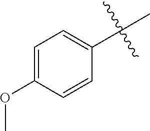 [SMILES: *c1ccc(OC)cc1] | H | 4,5-CH₂O₂ | 4.709 | >1000 |
| C301 | 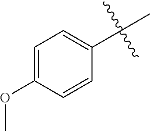 [SMILES: *c1ccc(OC)cc1] | 5-Bromo | 4,5-CH₂O₂ | 5.666 | >1000 |

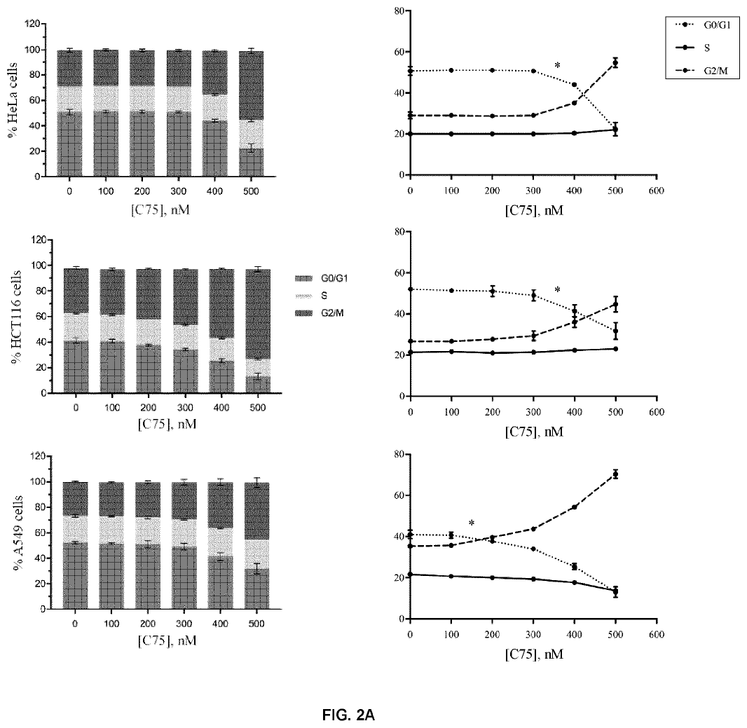

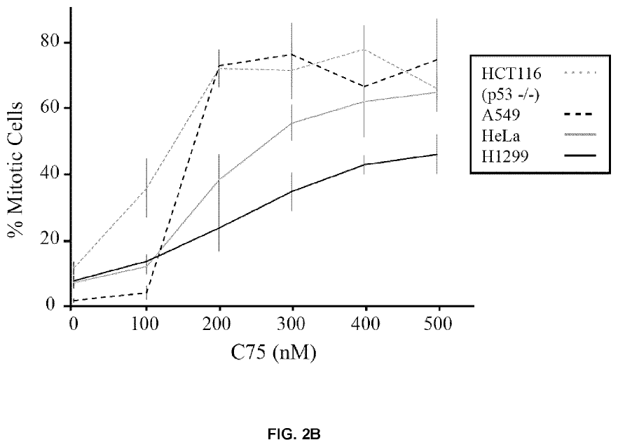

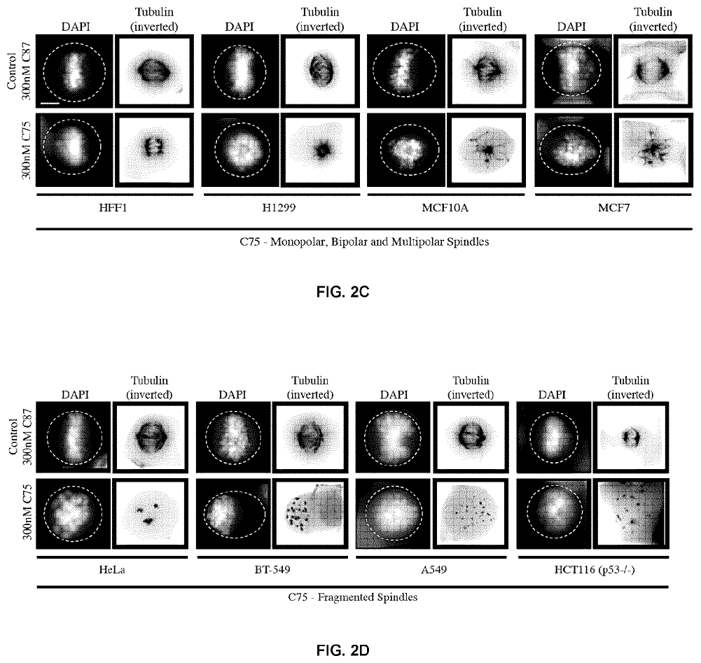

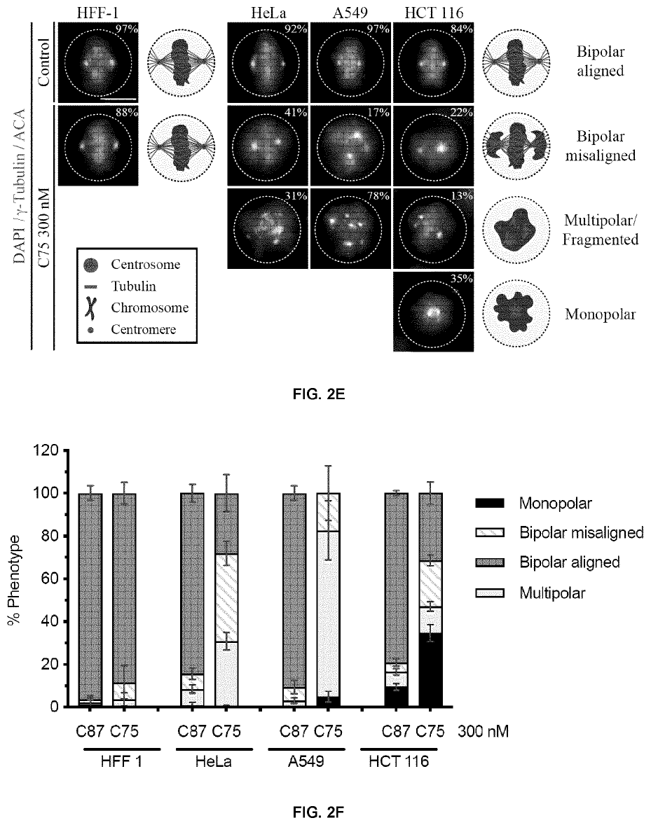

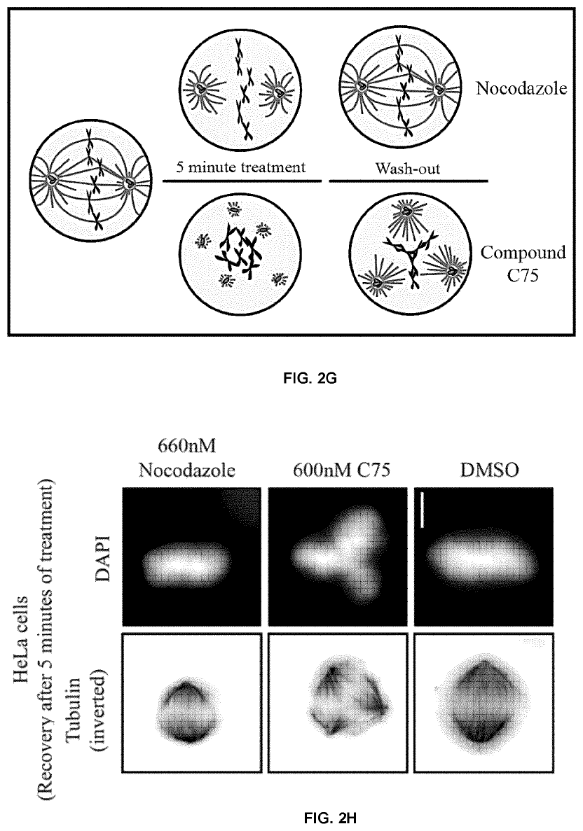

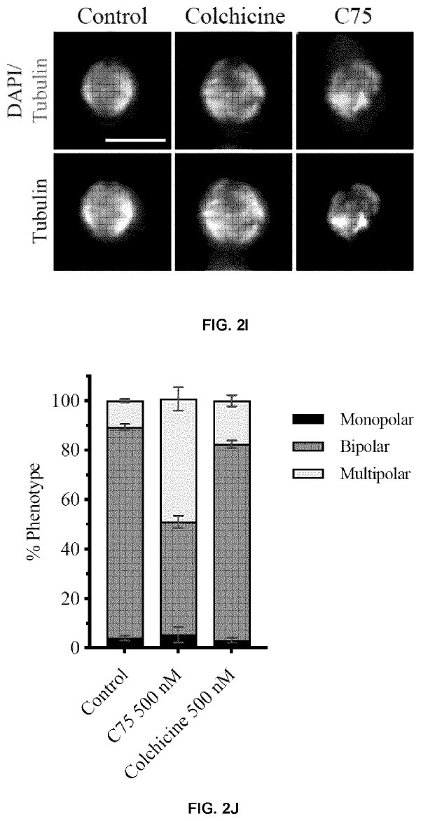

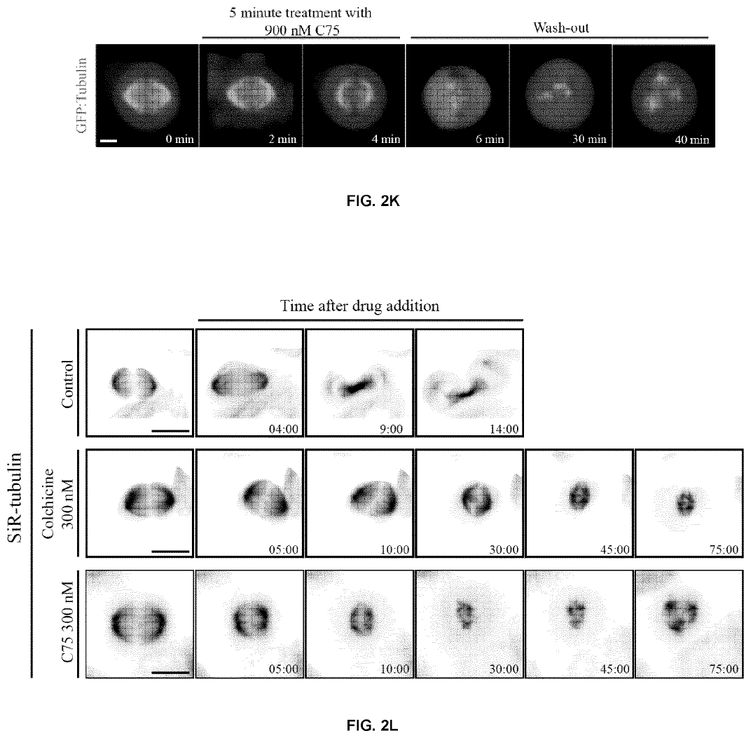

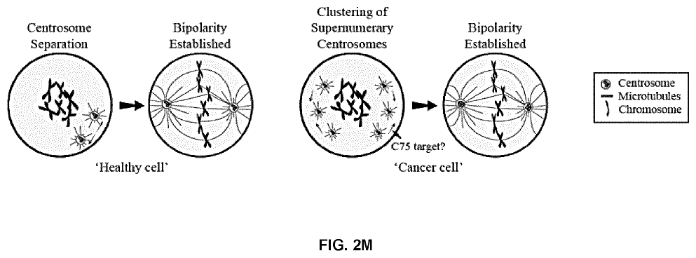

C75 was also tested for efficacy in different cancer cell lines. In HCT116 (colorectal cancer), HeLa (cervical cancer) cells, A549 (lung cancer) cells, C75 caused an increase in the proportion of G2/M cells after treatment with 300 nM or 400 nM for 8 hours (FIG. 2A). Longer treatments (e.g. one population doubling time) of HCT116, HeLa, A549 and H1299 (non-small cell lung cancer) cells caused mitotic arrest with 100-200 nM of C75 (FIG. 2B). HCT116 cells appeared to be most responsive (increase at 100 nM), while H1299 appeared to be the least responsive (small increase at 200 nM). In addition, C75 caused spindle phenotypes that varied depending on the cell type. While the majority of HFF1 cells had bipolar spindles after C75 treatment, H1299 cells, MCF-10A cells and MCF-7 cells, displayed a mix of monopolar, bipolar and multipolar spindles after C75 treatment (FIG. 2C). More severe spindle phenotypes were observed in HeLa cells, BT-549 cells, A549 cells and HCT116 cells after C75 treatment, where most of the spindles were severely fragmented (FIG. 2D). To further assess the role of C75 in disrupting spindles, HeLa cells were treated with 300 nM C75 for 4 hours and the spindle phenotypes were analyzed in more detail (FIG. 2E). While the majority of HFF-1 cells had bipolar spindles with aligned chromosomes (88%), all of the cancer cells showed more severe spindle phenotypes. The majority of HeLa, A549 and HCT116 cells had bipolar spindles with misaligned chromosomes (41%, 17% and 22%, respectively) and multipolar or fragmented spindles (31%, 78% and 13%, respectively). HCT116 cells also had a significant proportion of cells with monopolar spindles (35%). The proportion of spindle phenotypes for the control (C87-treated) vs. C75-treated cells is shown as a bar graph in FIG. 2F). These phenotypes show that C75 disrupts the spindle by affecting the centrosomes. To assess the affect of C75 on centrosomes, HeLa cells were treated with C75 or nocodazole (a drug that causes microtubule depolymerization as a control) for a short period of time (5 minutes), then the drugs were removed and the cells were analyzed for spindle phenotypes (schematic in FIG. 2G). While in cells treated with nocodazole, bipolar spindles reformed, in cells treated with C75, tri- or multipolar spindles formed (FIG. 2H). This was repeated for comparison with another microtubule depolymerizing drug called colchicine, which had a similar outcome (FIG. 2I). The proportion of cells with multipolar spindles that reformed after drug removal is significantly greater in cells treated with C75 in comparison to those treated with colchicine (FIG. 2J). In addition, live imaging revealed that within 4-6 minutes after exposure to C75, the centrosomes fragmented or declustered and microtubules rapidly collapsed with no visible polymers remaining. Microtubule polymers grew back within ˜20-30 minutes of washout, but the spindles were tri- or multipolar (FIG. 2K). Even without washout of C75, live imaging of HeLa cells treated with C75 revealed that microtubule polymers grow back to form multipolar spindles, while polymers remain collapsed in cells treated with colchicine (FIG. 2L). This emphasizes the ability of thienoisoquinoline compounds to affect the centrosomes in comparison to other compounds that affect mitosis by targeting tubulin (FIG. 2M).

### Example 2

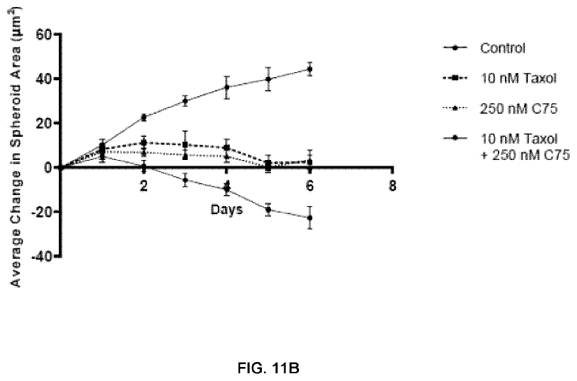

The thienoisoquinoline compounds may also have potential for use in combinatorial therapies. Several known anti-cancer drugs cause mitotic arrest by disrupting microtubule dynamics, and have been used to combat a spectrum of cancers, including paclitaxel (Taxol™) and vinblastine^{[12,13]}. Taxanes (e.g. paclitaxel), vinca alkaloids (e.g. vinblastine) and other drugs such as colchicine and nocodazole bind to β-tubulin or to the α-β-tubulin lattice. Evidence that C75 can be used in combination with other anti-cancer drugs and enhances the effects of tubulin-targeting drugs is in FIGS. 11A) and 11B). We observed that adding threshold concentrations of paclitaxel and C75 in combination to HCT116 spheroids causes them to regress in size vs. each on their own, which halts, but is not sufficient to regress growth. We are currently investigating the use of C75 in combination with other anti-cancer drugs in spheroids.

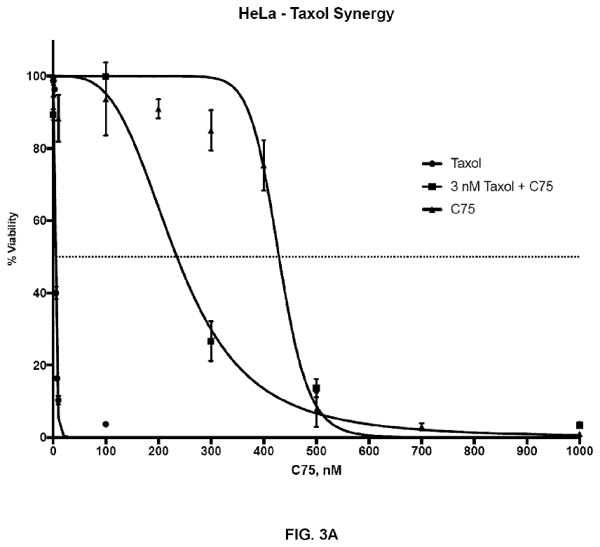

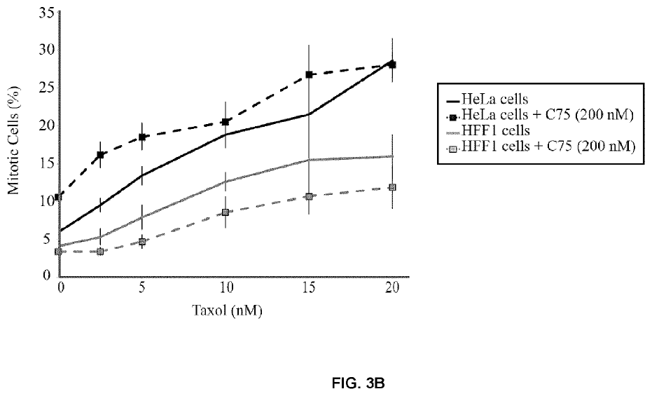

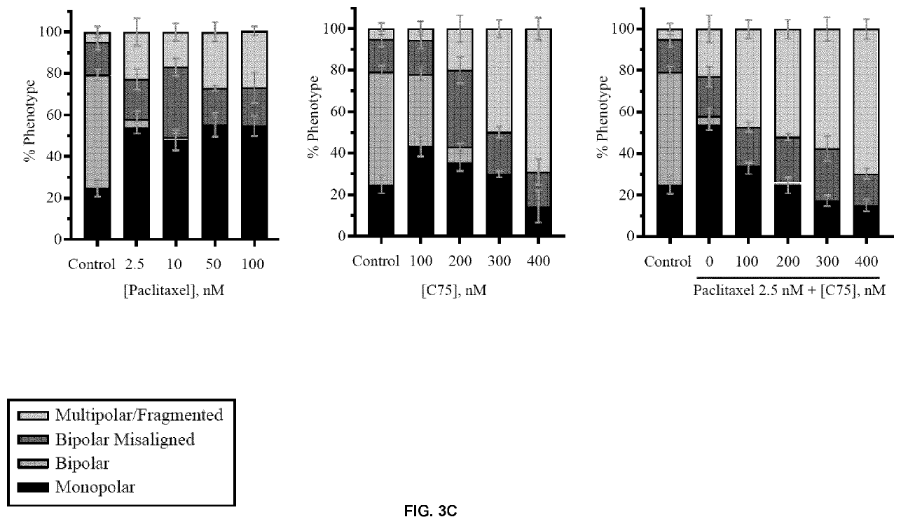

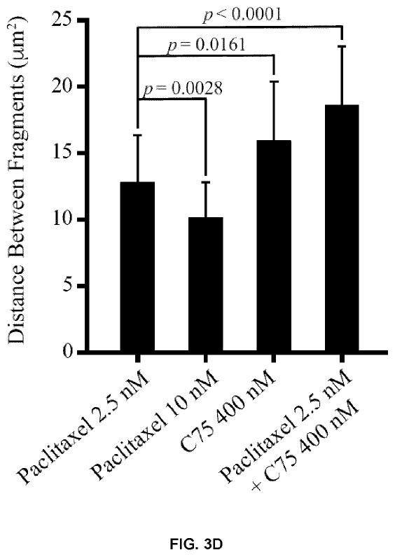

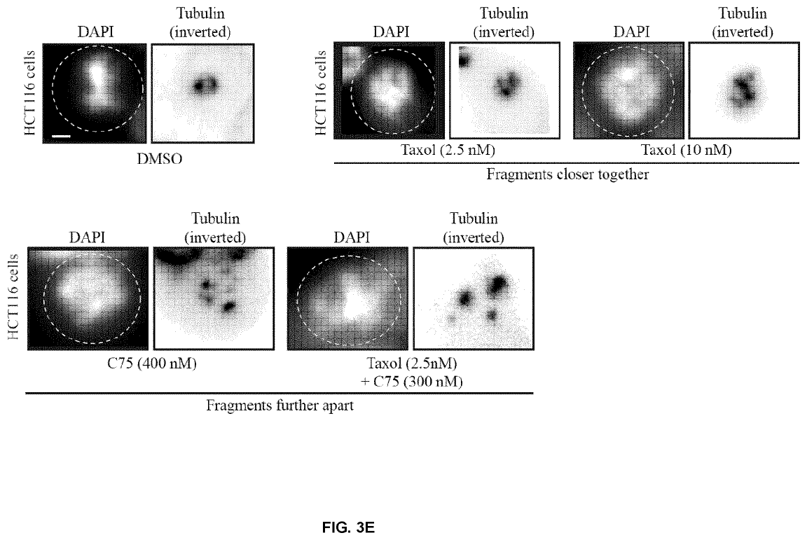

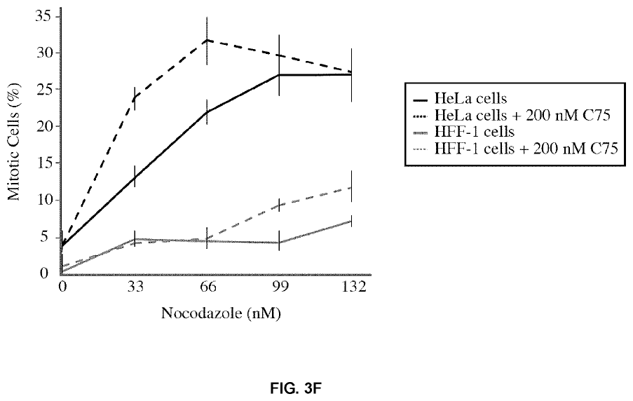

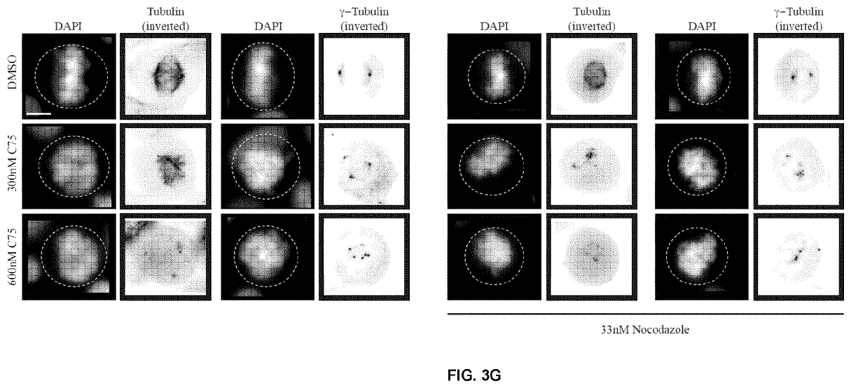

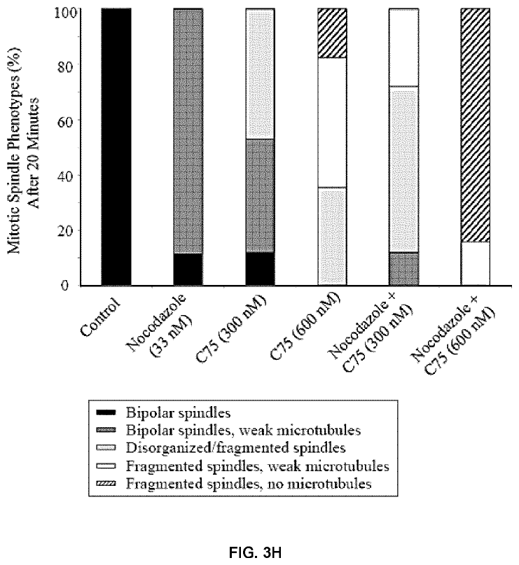

Typically, α-tubulin forms a dimer with β-tubulin that then assembles into the polymers that make microtubules^{[30]}. At low concentrations, tubulin-targeting drugs stabilize microtubules without changing their polymer mass, effectively ‘freezing’ the mitotic spindle. C75 does not appear to do this and enhances the efficacy and selectivity of paclitaxel in HeLa cells vs. HFF1 cells (e.g. FIG. 3A-E). Adding a subthreshold dose of paclitaxel that causes no toxicity enhances the cytotoxic effects of C75 in HeLa cells and reduces the IC₅₀ of C75 by ˜2-fold (FIG. 3A). In addition, it was found that C75 offers a protective effect to non-cancerous HFF1 cells treated with paclitaxel (FIG. 3B). C75 enhances the phenotypes caused by paclitaxel in HCT116 cells (FIG. 3C-E). The proportion of mitotic spindle phenotypes is thresholded with 2.5 nM paclitaxel, and despite this, adding increased amounts of C75 worsens the phenotypes, suggesting that C75 has a unique molecular target vs. paclitaxel (FIG. 3C). Also, there was a significant decrease in fragment distance with increased paclitaxel concentration, but a significant increase in fragment distance when paclitaxel was combined with C75 (FIGS. 3D and 3E).

An in-depth analysis of HeLa cells treated with both C75 and nocodazole have higher efficacy and selectivity for cancer cells, and have more extreme phenotypes in HeLa cells when treated together vs. on their own (FIG. 3F-H). For example, there is no change in the proportion of mitotic HFF1 (non-cancer) cells when treated with a low dose of nocodazole (e.g. 33 or 66 nM) in combination with 200 nM C75, but there is a large change in HeLa cells. Also, HeLa cells treated with nocodazole alone exhibit bipolar spindles with weak microtubules and HeLa cells treated with C75 alone have disorganized and/or fragmented mitotic spindles, whereas cells treated with both drugs exhibit increased fragmentation and loss of microtubules (FIG. 3H). Therefore, thienoisoquinoline compounds such as C75 may have the potential for use in combinatorial therapies.

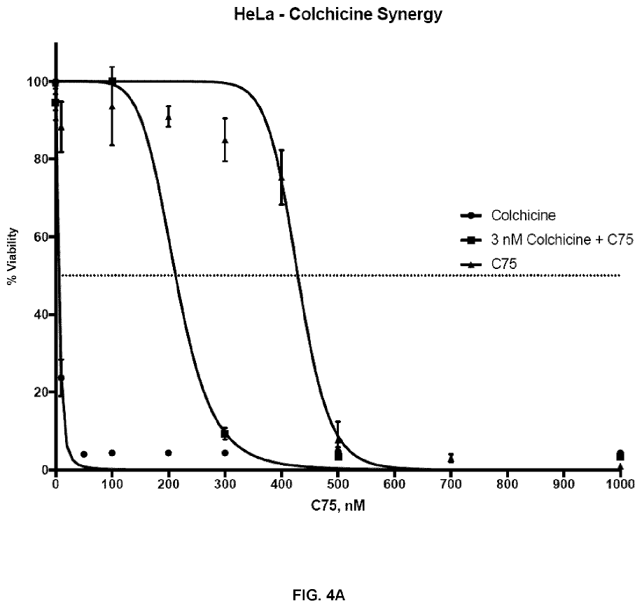

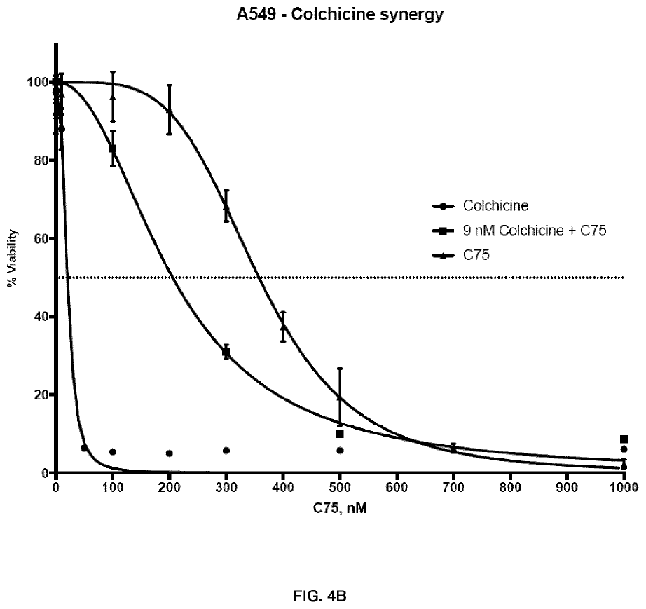

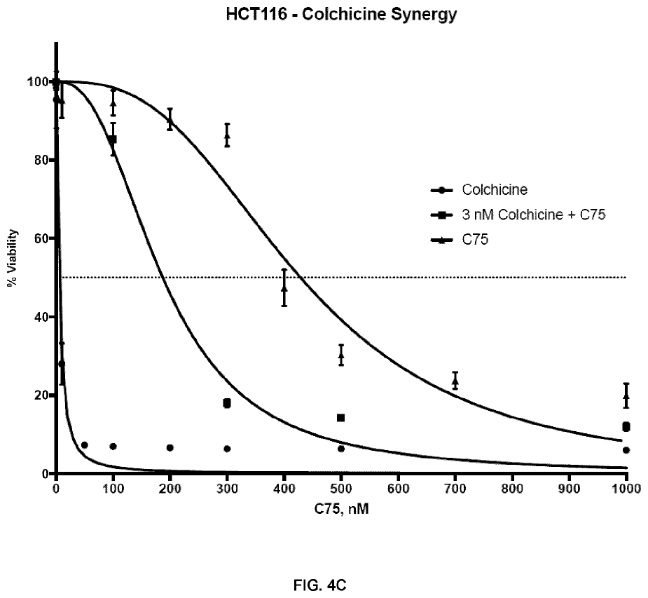

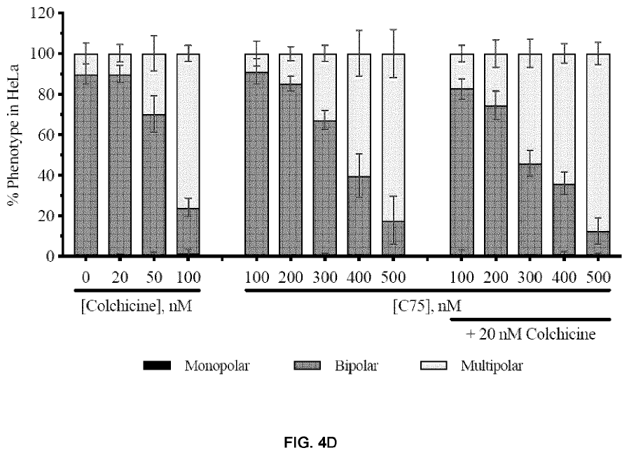

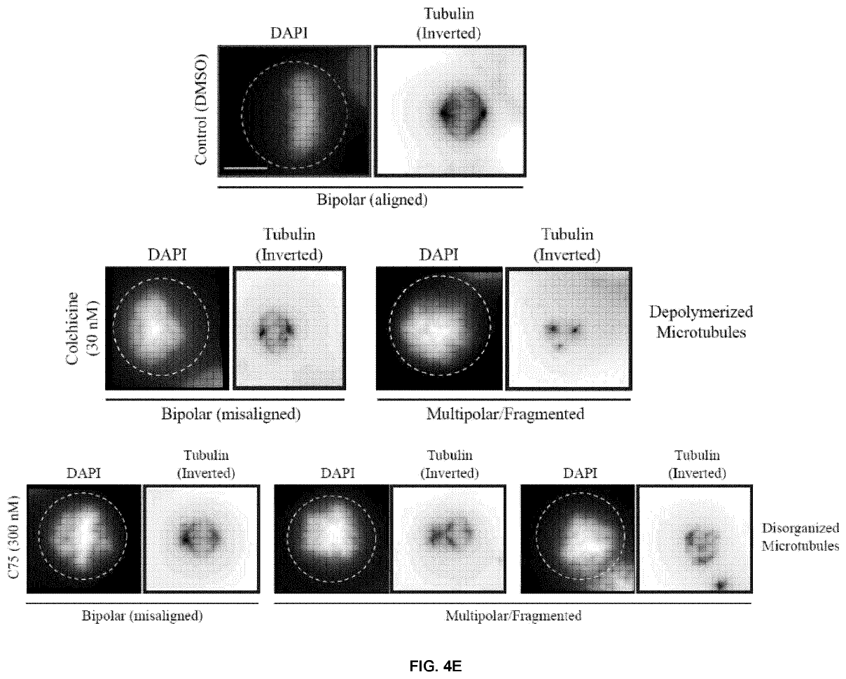

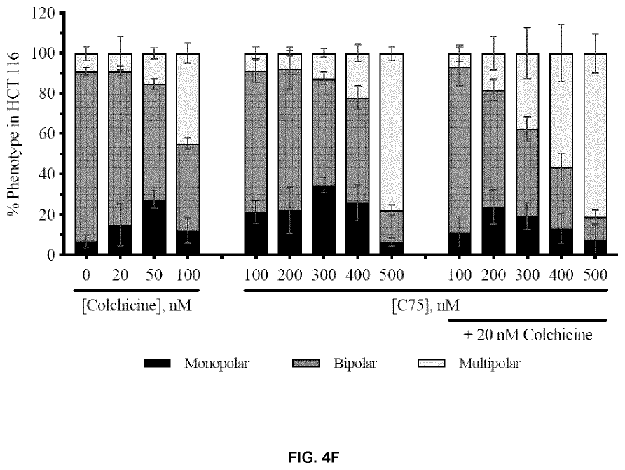

HeLa, A549 and HCT116 cells treated with subthreshold, non-toxic doses of colchicine in combination with C75 lowers its IC50 by ˜2-fold, and adding a low dose of colchicine enhances the mitotic spindle phenotypes caused by C75 in HeLa and HCT116 cells (FIG. 4A-F). This enhanced toxicity shows that C75 could be used in combination therapies with microtubule depolymerizing drugs.

In addition, the thienoisoquinoline compounds disclosed herein may have the potential for use in combinatorial therapies with non-mitotic anti-cancer drugs (non-tubulin-targeting drugs) such as doxorubicin, an anthracycline, an alkylating drug, or an antimetabolite. Specifically, by not targeting tubulin or by targeting tubulin differently, and still disrupting mitosis, the thienoisoquinoline compounds may be used as an anti-mitotic agent in cells that are resistant to tubulin-targeting drugs such as taxanes (e.g. due to the upregulation of alternate beta-tubulin isoforms). To evaluate this, cancer cell lines resistant to tubulin-targeting drugs or having higher resistance to tubulin-targeting drugs may be treated with a thienoisoquinoline compound in combination with a non-mitotic anti-cancer drug, for example doxorubicin. Efficacy, for example in terms of mitotic arrest, may be evaluated using the methods described in Example 1.

### Example 3

The anti-mitotic activity of a thienoisoquinoline compound can be evaluated in vivo using subcutaneous xenografts in rodents. Human cancer cells, e.g. obtained from patients and/or from cancer cell lines, are injected into each of the lower legs (one as a control) of nude rats or SCID mice. Other types of grafting in animal models to test the compounds, e.g. colorectal cancers with different metastases, are also contemplated. Different regiments of treatment will be tested by injection before tumors form, or after ˜2 weeks when tumors are palpable (e.g. 50 mm³). The treatment may also be administered orally, in particular to test for safety and bioavailability. The rodents are treated daily, after every 2-3 days, or weekly with an effective amount of a thienoisoquinoline compound or saline (control) under suitable conditions for a determined duration. Tumors will be monitored daily for change in growth (% treated vs. control). At the end of the study, tumors will be collected and fixed for more in depth analyses of tumor morphology. Toxicity will be monitored by weight loss and/or death^{[31-33]}.

### Example 4

The anti-mitotic effect as well as synergistic and protective effects of a thienoisoquinoline compound in combination with an anti-cancer agent such as for example a taxane compound or vinca alkaloid, can also be assessed in vivo using similar methods as described in Example 3.

### Example 5

To show that C75 has a different effect on the mitotic spindle compared to microtubule polymerizing agents, toxicity, mitotic arrest and spindle phenotypes were compared in HCT116 cells after treatment with C75+/− paclitaxel (FIG. 3A-D). For example in FIG. 3A, a subthreshold dose of paclitaxel that has no toxicity in HeLa cells enhances toxicity caused by C75. Adding 200 nM of C75 increases the efficacy and selectivity of paclitaxel in cancer (HeLa) vs. non-cancer cells (HFF1; FIG. 3B). In addition, adding a threshold dose of paclitaxel in combination with C75 worsens mitotic spindle phenotypes more vs. each on their own (FIG. 3C) and causes unique spindle phenotypes (FIG. 3D). This data shows that C75 has a different molecular target or binds tubulin at a different binding site than paclitaxel.

To show that C75 has a different effect on the mitotic spindle compared to microtubule depolymerizing agents, the spindle phenotypes were compared in HeLa cells after treatment with C75+/− nocodazole or colchicine, both of which target tubulin and induce microtubule depolymerization. As shown in FIGS. 3F-H and 4A-D, nocodazole and colchicine each cause enhanced toxicity, mitotic arrest and spindle phenotypes when added with C75 vs. on their own. For example, in FIG. 4A), Fig. B) and FIG. 4C), subthreshold doses of colchicine that do not cause toxicity in HeLa, A549 or HCT116 cells enhance C75 and decrease its IC₅₀ by ˜2-fold. As shown in FIGS. 4D) and 4F), a subthreshold dose of colchicine (20 nM) enhances spindle phenotypes caused by 300 nM and/or 400 nM of C75 in HeLa and HCT116 cells after 5 hours. Comparing spindle phenotypes caused by colchicine vs. C75 in FIG. 4E) shows how spindles remain bipolar, but have fewer microtubules after colchicine treatment at low concentrations, while C75 causes disorganized and/or fragmented spindles. At high concentrations, both drugs can cause fragmented spindles. These data support that C75 has either a different molecular target or binds tubulin, but at a different site vs. nocodazole or colchicine.

### Example 6—Testing in Tumour Spheroids

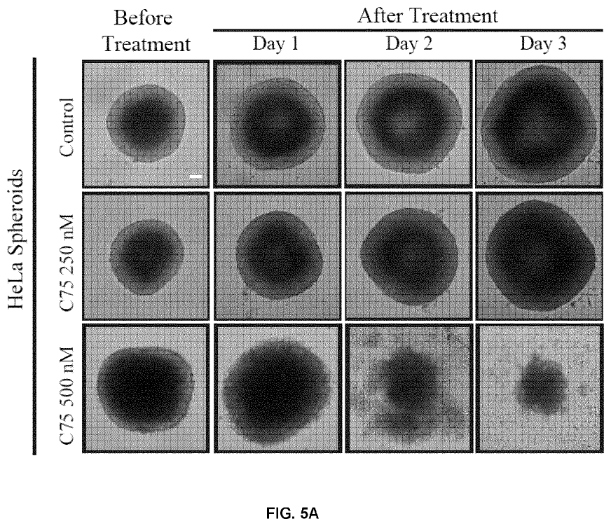

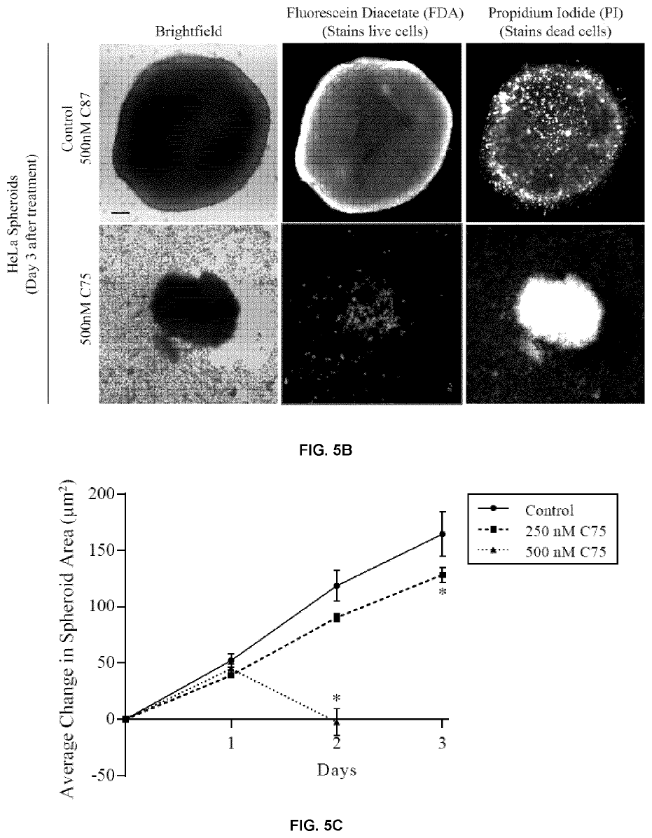

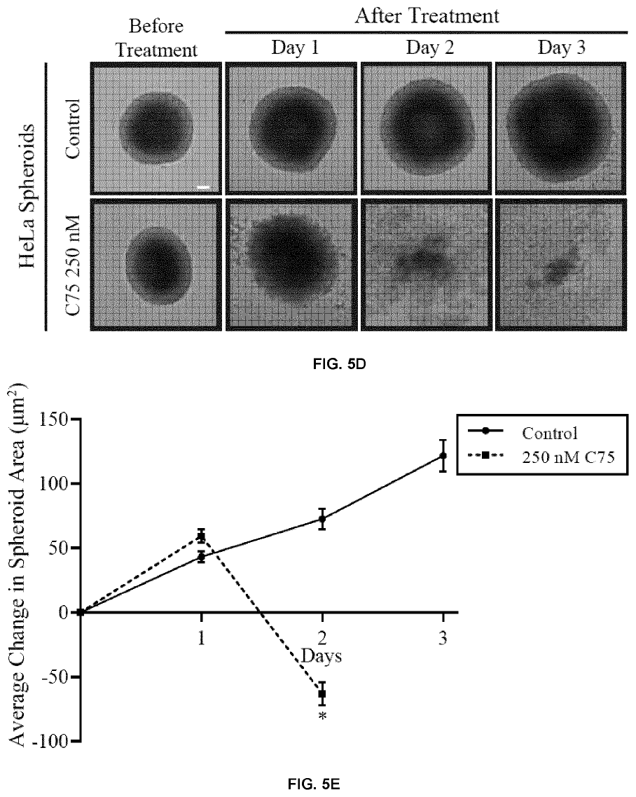

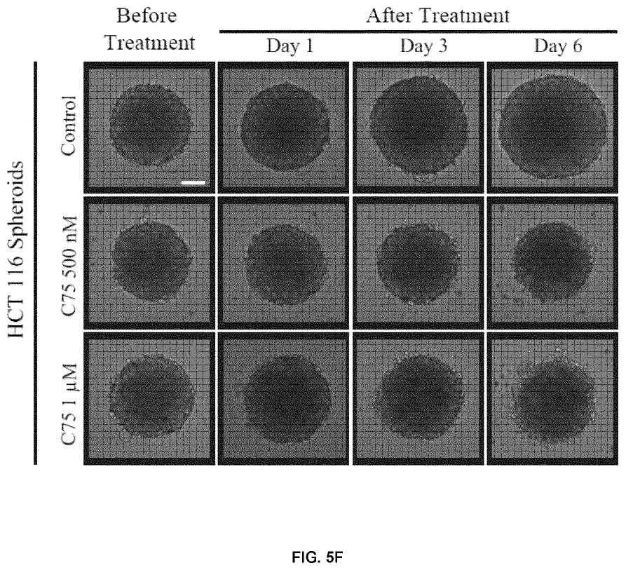

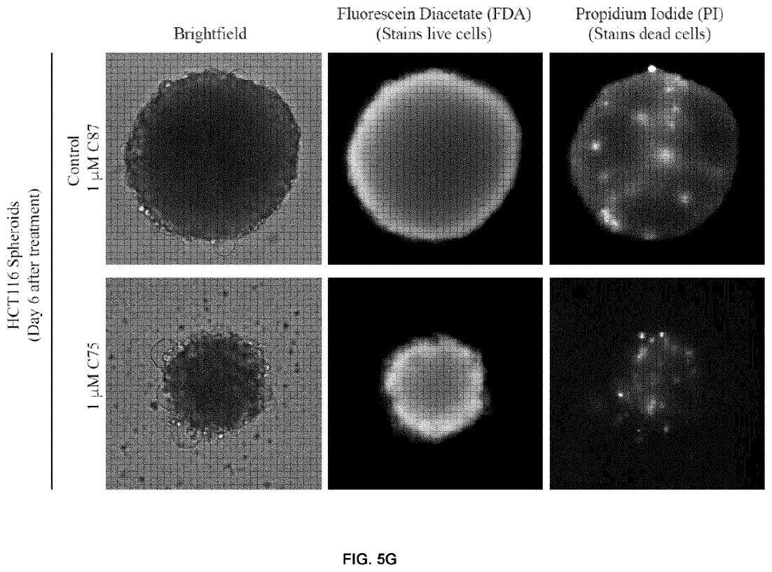

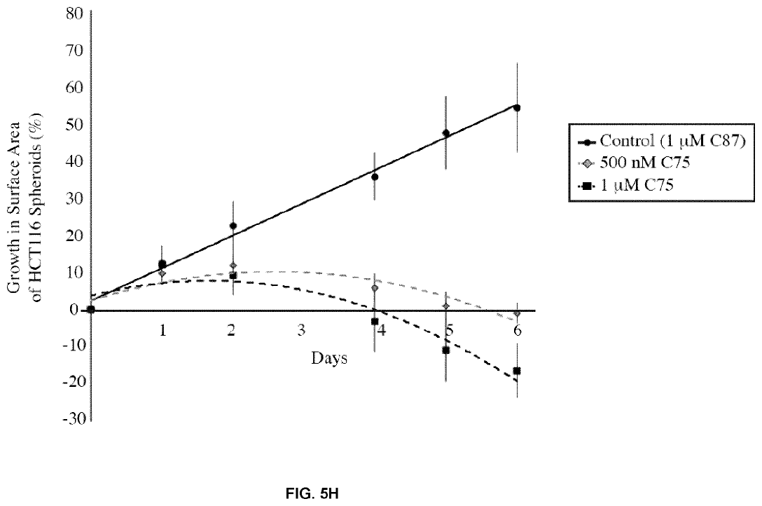

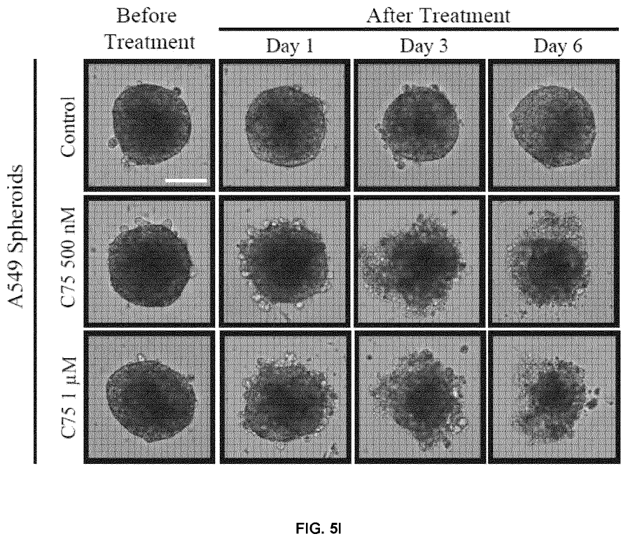

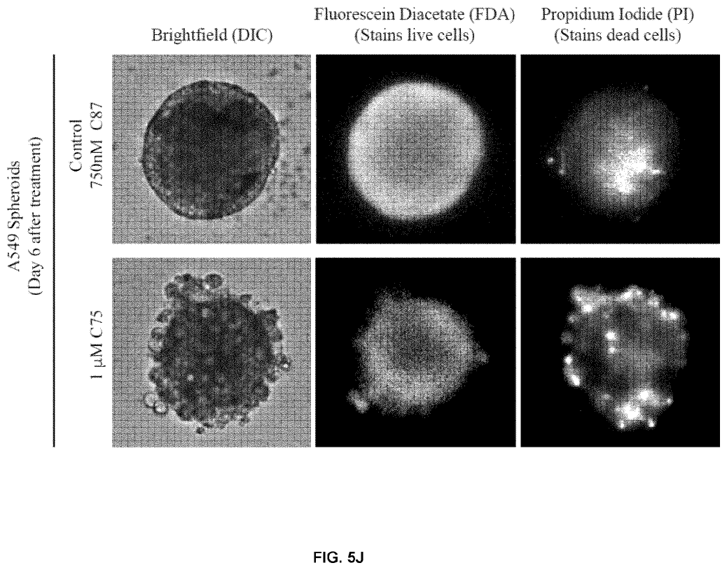

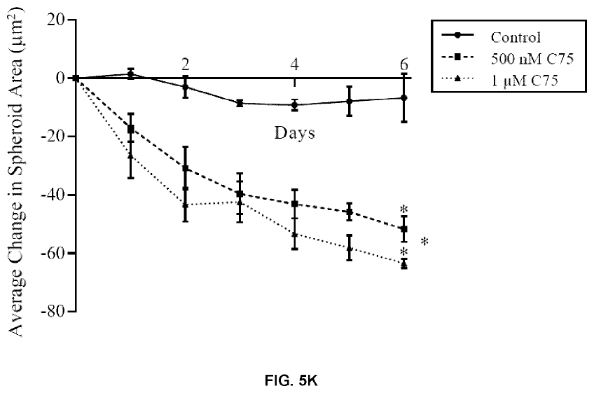

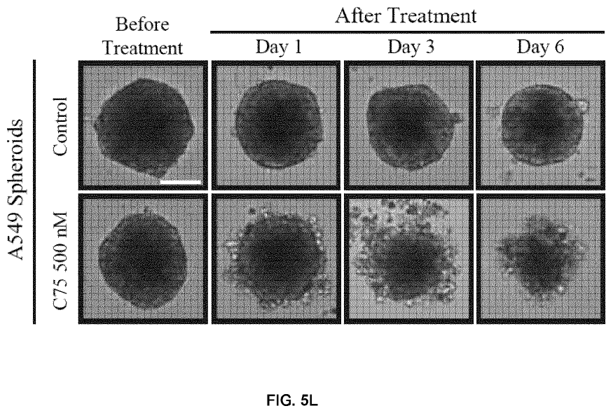

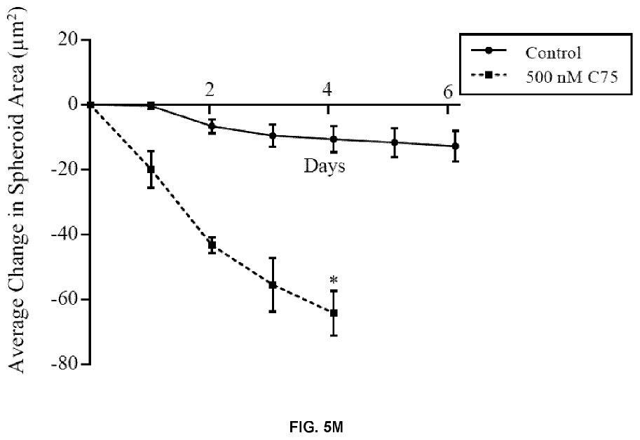

C75 was further tested in multicellular tumour spheroids, which are more representative of in vivo tumours in comparison to growing cells as monolayers. These spheroids were produced according to the methods of Friedrich et al. 2009 [34]. Briefly, 96-well plates coated with 1.5% agarose were seeded with 500-1000 HeLa or HCT116 cells as outlined for the liquid-overlay technique. They were left to aggregate with gravity in optimal growth conditions, and individual spheroids were transferred to 24-well dishes for further growth and treatment. A549 spheroids were initiated using the hanging-drop method outlined by Froehlich et al. 2016^{[35]}, and transferred to 24-well dishes for further growth and treatment. HeLa, HCT116 and A549 spheroids were treated with C75 alone, or C75-loaded into biodegradable polymeric nanoparticles. Nanoparticles can be produced for example according to the methods reviewed in Zhang et al. 2012^{[36]} and Hong et al. 2018^{[37]}. Both C75 alone and C75-loaded nanoparticles were shown to disrupt HeLa spheroids (FIGS. 5A, 5B, 5C, 5D, and 5E), and regress the growth of HCT116 spheroids (FIGS. 5F, 5G, and 5H) and A549 spheroids (FIGS. 5I, 5J, 5K, 5L, and 5M).

### Example 7—New Synthetic Route

Another novel synthesis for thienoisoquinoline scaffold derivatives was designed.

### C-75 Derivative Synthesis Experimental

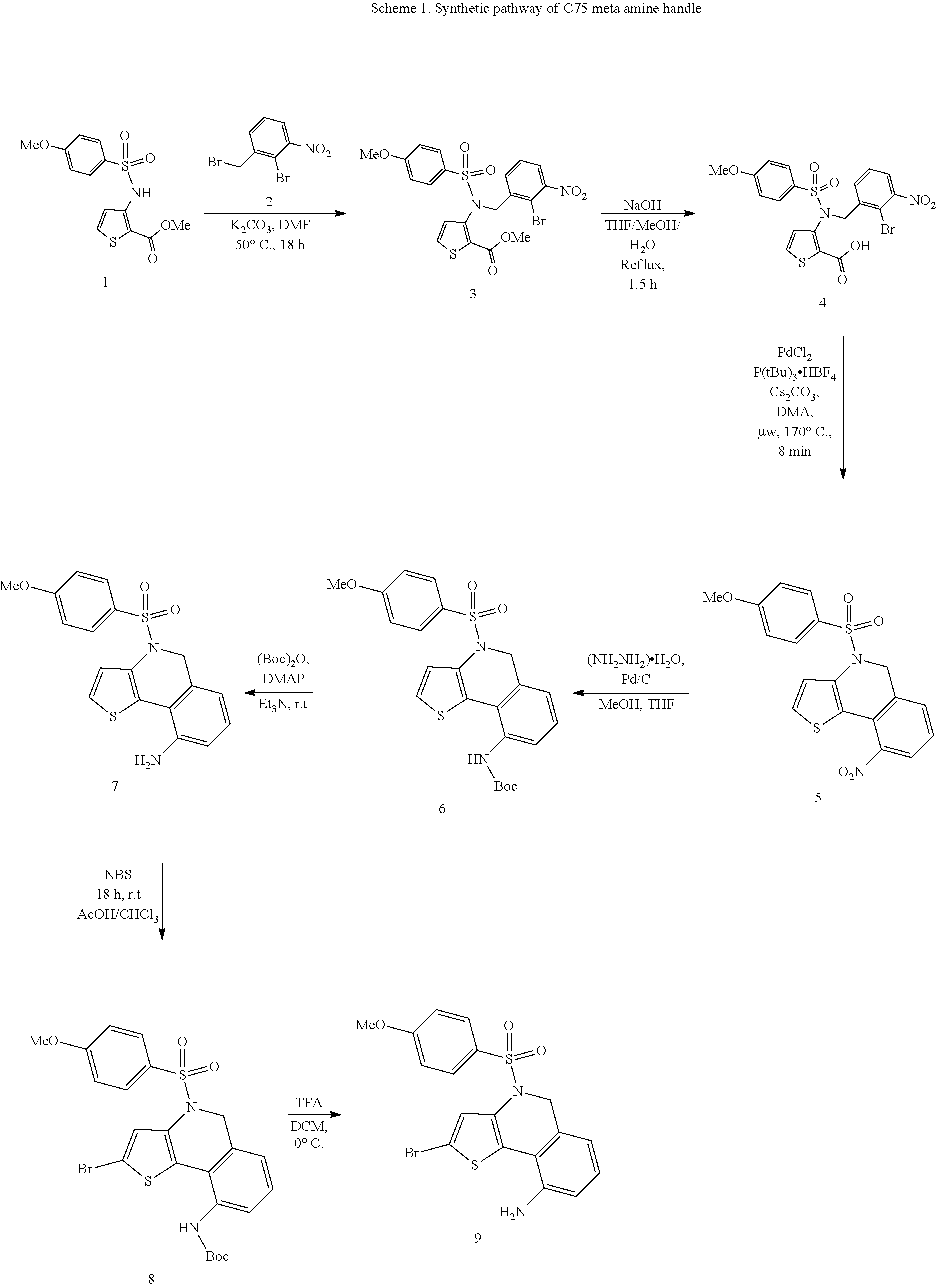

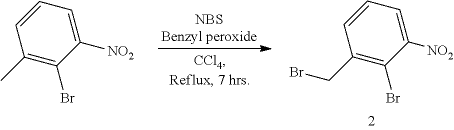

General Procedure for Bromination of Nitrotoluene

2-bromo-1-methyl-3-nitrobenzene (1 equiv.) and NBS (1.1 equiv) are mixed in 0.8M of anhydrous CCl₄ in an oven-dried vessel. The mixture is purged with Argon gas, and heated under reflux for 7 hours. The mixture is cooled to room temperature and diluted with DCM, then follow by washing with distilled water. The Aqueqous layer is extract with DCM. The combined organic layers are washed three times with distilled water, and dried over Na₂SO₄. The compound is purified with column chromatography. The product is white solid. Isolated yield 72%.

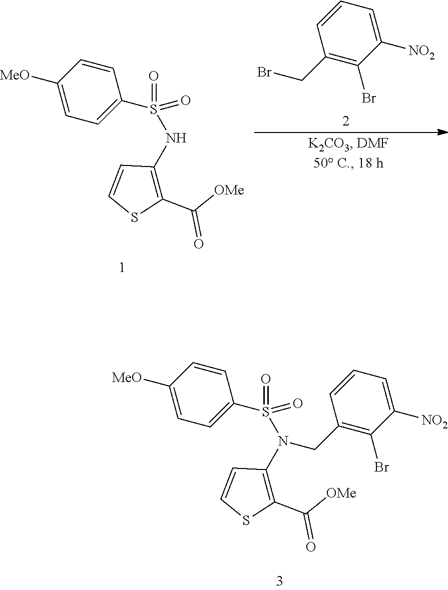 [SMILES: COC(=O)c1sccc1NS(=O)(=O)c1ccc(OC)cc1 ; O=[N+]([O-])c1cccc(CBr)c1Br ; COC(=O)c1sccc1N(Cc1cccc([N+](=O)[O-])c1Br)S(=O)(=O)c1ccc(OC)cc1]

General Procedure for Benzylation

Methyl 3-((4-methoxyphenyl)sulfonamido)thiophene-2-carboxylate (1 equiv.), 2-bromo-1-(bromomethyl)-3-nitrobenzene (1.2 equiv.), and K₂CO₃ (3 equiv.) are mixed in 0.3M of DMF. Solution is heated to 50° C. for 18 hours. Reaction mixture is cooled to room temperature after 18 hours. The mixture is diluted with EtOAc and washed with distilled water, and aqueous layer is extracted with EtOAc. The combined organic layers are washed with distill water three times, saturated salt solution three times, and dried over Na₂SO₄ The solvent is evaporated under reduced pressure, and solid residue is recrystallized with EtOAc and hexane. The final product is pale yellow crystal. Isolated yield: 92%

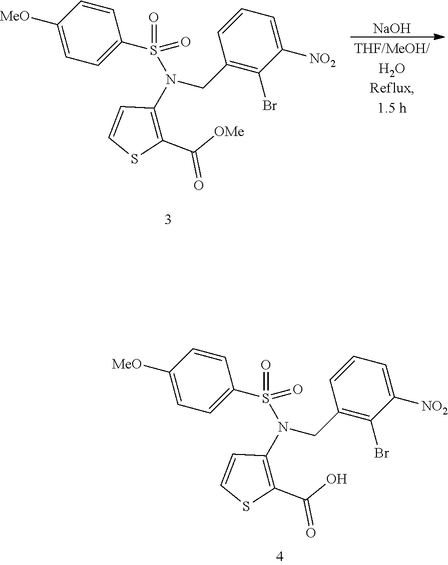 [SMILES: COC(=O)c1sccc1N(Cc1cccc([N+](=O)[O-])c1Br)S(=O)(=O)c1ccc(OC)cc1 ; "NaOH" ; COc1ccc(S(=O)(=O)N(Cc2cccc([N+](=O)[O-])c2Br)c2ccsc2C(=O)O)cc1]

General Procedure for Saponification

Methyl 3-((N-(2-bromo-3-nitrobenzyl)-4-methoxyphenyl)sulfonamido)thiophene-2-carboxylate (1 equiv.) and NaOH powder (5 equiv.) was mixed and dissolved with THF, water, and MeOH (2:1:1 respectively, 0.1M overall). The mixture is heated under reflux, then cooled to room temperature after 1.5 hours for completion, TLC is used to monitor the reaction. The mixture is treated with 1M HCl solution until pH reaches 1. The mixture is diluted with distilled water and extracted with DCM two times. The combined organic layers are washed with water three time, saturated salt solution two times, and dried over Na₂SO₄. The solvent is evaporated under reduced pressure, and residue is recrystallized with EtOAc and hexane. The final product is yellow solid. Isolated yield: 95%

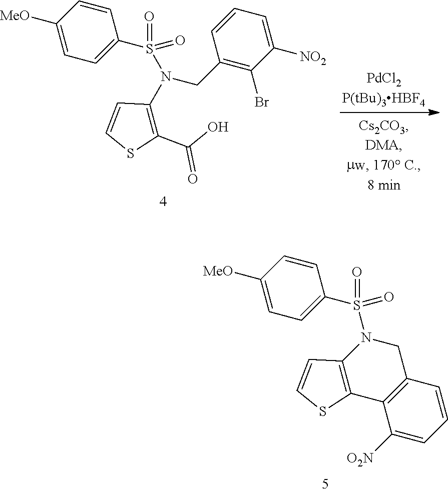 [SMILES: COc1ccc(S(=O)(=O)N(Cc2cccc([N+](=O)[O-])c2Br)c2ccsc2C(=O)O)cc1 ; COc1ccc(S(=O)(=O)N2Cc3cccc([N+](=O)[O-])c3-c3sccc32)cc1]

General Procedure for Decarboxylative Cross-Coupling

3-((N-(2-bromo-3-nitrobenzyl)-4-methoxyphenyl)sulfonamido)thiophene-2-carboxylic acid (1 equiv.) in DMA (0.1M) was added to an oven-dried microwave vial contain PdCl₂ (0.1 equiv.), P(tBu)₃.HBF₄ (0.2 equiv.), Cs₂CO₃ (3 equiv.). The mixture is heated under microwave radiation at 170° C. for 8 min, then cooled to room temperature, and diluted with EtOAc. The mixture is washed with distilled water, and aqueous layer is extracted with EtOAc. The combined layers are washed with saturated NaHCO₃, distilled water, saturated NaCl solution, and dried over Na₂SO₄. The solvent is evaporated under reduced pressure. The product is purified by column chromatography and recrystallization from CHCl₃ and MeOH. The final product is yellow crystal. Isolated yield: 41%.

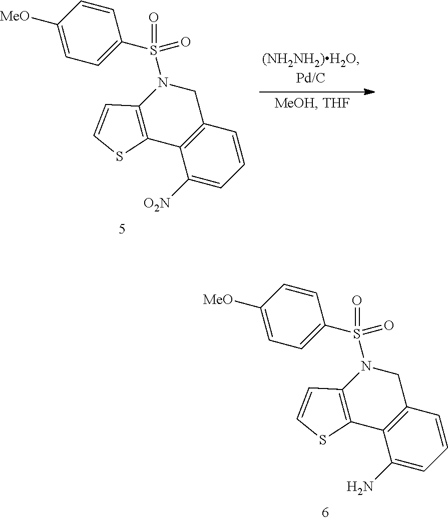 [SMILES: COc1ccc(S(=O)(=O)N2Cc3cccc([N+](=O)[O-])c3-c3sccc32)cc1 ; COc1ccc(S(=O)(=O)N2Cc3cccc(N)c3-c3sccc32)cc1]

General Procedure for Reduction

4-((4-methoxyphenyl)sulfonyl)-9-nitro-4,5-dihydrothieno[3,2-c]isoquinoline (1 equiv.) and Pd/C (0.1 equiv. 10 mol %) is mixed with THF and MeOH (1:1 ratio, 0.2M overall). Hydrazine hydrate (10 equiv.) is added slowly to the mixture. The mixture is heated under reflux for 20 mins for completion. TLC is used to monitor the reaction. Mixture is filtered from a pad of celite and washed with EtOAc. The mixture is washed with distilled water, and aqueous layer is extracted with EtOAc, The combined organic layer is washed with distilled water one time, saturated salt solution one time, and dried over Na₂SO₄. The solvent is evaporated under reduced pressure. The residue is recrystallized with CHCl₃ and MeOH. Isolated yield: 70%.

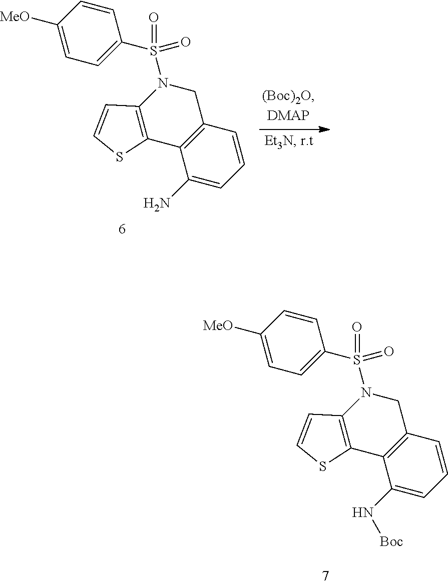 [SMILES: COc1ccc(S(=O)(=O)N2Cc3cccc(N)c3-c3sccc32)cc1 ; COc1ccc(S(=O)(=O)N2Cc3cccc(NC(=O)OC(C)(C)C)c3-c3sccc32)cc1]

General Procedure for Amine Protection

4-((4-methoxyphenyl)sulfonyl)-4,5-dihydrothieno[3,2-c]isoquinolin-9-amine (1 equiv.) and Di-tert-butyl dicarbonate (2 equiv.) are dissolved in Et₃N (0.4 M), DMAP (1 equiv.) is added into mixture. The mixture is placed under room temperature for 48 hours. The mixture is diluted with EtOAc and washed with distilled water. The aqueous layer is extracted with EtOAc. The combined organic layers are washed with 1M HCl one time, distilled water three times, saturated salt solution one time, and dried over Na₂SO₄. The solvent is evaporated under reduced pressure. The product is purified by column chromatography. The product is colorless solid. Isolated yield: 60%

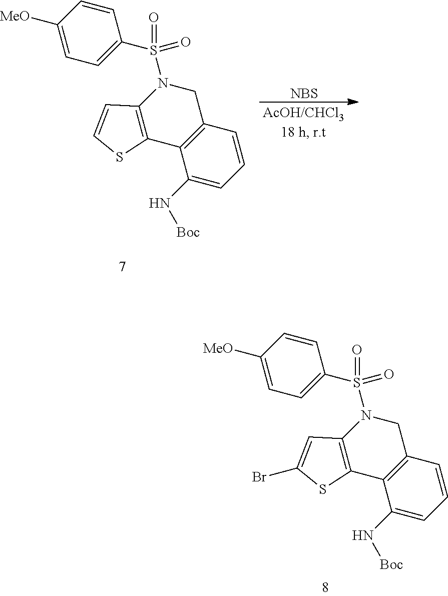 [SMILES: COc1ccc(S(=O)(=O)N2Cc3cccc(NC(=O)OC(C)(C)C)c3-c3sccc32)cc1 ; "NBS" ; COc1ccc(S(=O)(=O)N2Cc3cccc(NC(=O)OC(C)(C)C)c3-c3sc(Br)cc32)cc1]

General Procedure for Bromination

tert-butyl (4-((4-methoxyphenyl)sulfonyl)-4,5-dihydrothieno[3,2-c]isoquinolin-9-yl)carbamate (1 equiv.) and NBS (1.1 equiv.) are mixed with CHCl₃ (0.1M) in an ember vial, and mixture is placed in ice bath follow by 2 v/v % AcOH. The reaction is slowly return to room temperature and run for 18 hours. The mixture is diluted with EtOAc then washed with distilled water, and aqueous layer is extracted with EtOAc. The combined organic layer is washed with distilled water one time, saturated salt solution one time, and dried over Na₂SO₄. The solvent is evaporated under reduced pressure. The product is purified by column chromatography. The product is light brown solid. Isolated yield: 30%

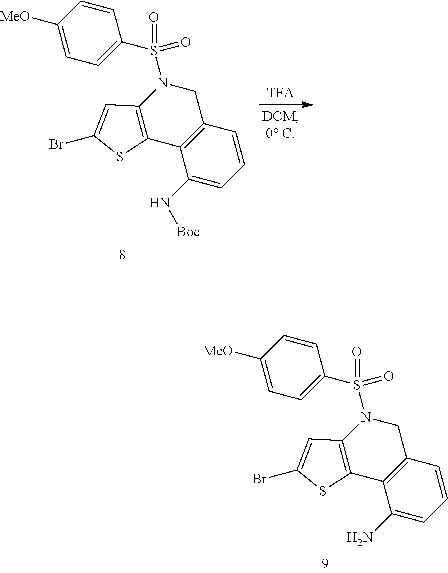 [SMILES: COc1ccc(S(=O)(=O)N2Cc3cccc(NC(=O)OC(C)(C)C)c3-c3sc(Br)cc32)cc1 ; COc1ccc(S(=O)(=O)N2Cc3cccc(N)c3-c3sc(Br)cc32)cc1]

General Procedure for Boc Group Deprotection

tert-butyl (2-bromo-4-((4-methoxyphenyl)sulfonyl)-4,5-dihydrothieno[3,2-c]isoquinolin-9-yl)carbamate (1 equiv.) is dissolved on DCM (0.1M) and placed in ice bath. 50 v/v % TFA is added slowly to mixture. The reaction is monitored by TLC, and stopped after 5 hours. The mixture is diluted with EtOAc then washed with distilled water, and aqueous layer is extracted with EtOAc. The combined organic layer is washed with saturated NaHCO₃ one time, distilled water one time, saturated NaCl solution one time, and dried over NaₛSO₄. The solvent is evaporated under reduced pressure. The product is purified by column chromatography. The product is brown solid. Isolated yield: 25%

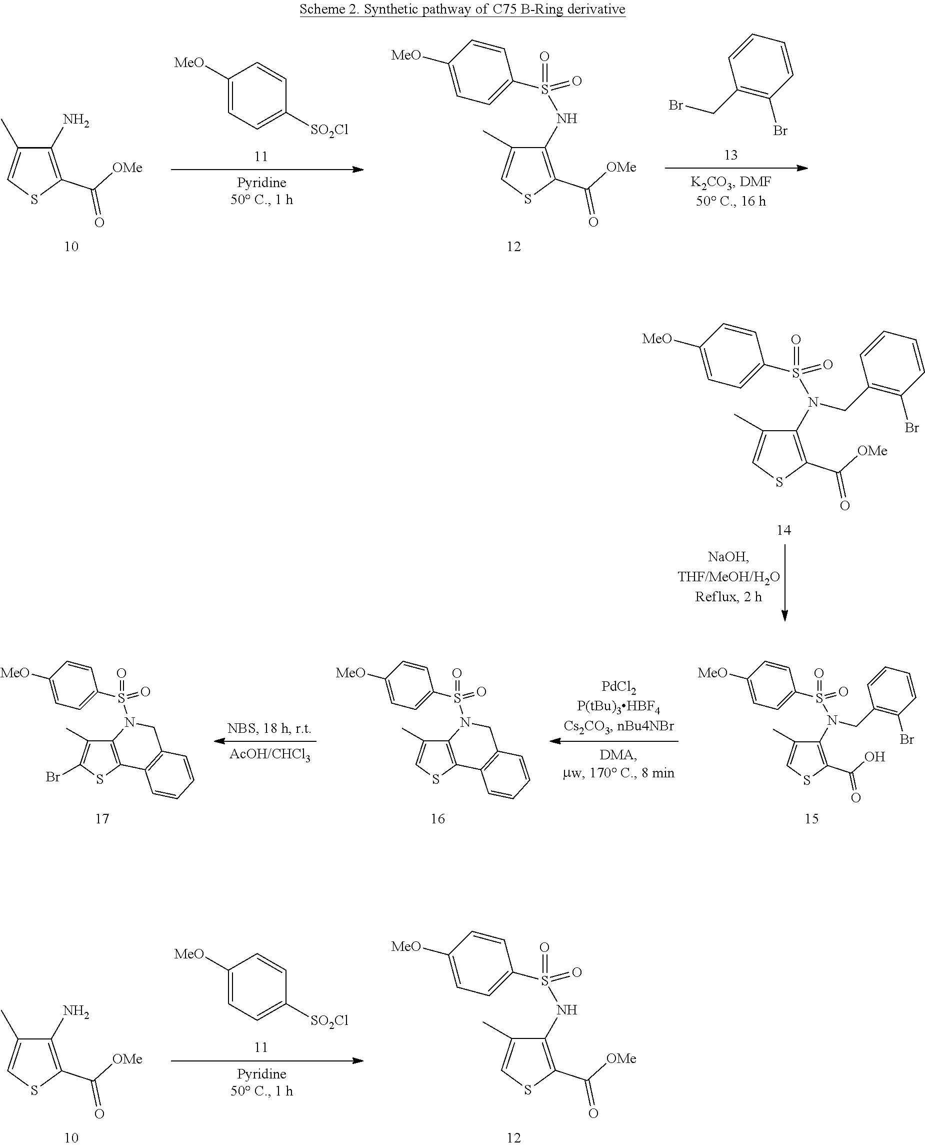

General Procedure for Sulfonylation

Methyl 3-amino-4-methylthiophene-2-carboxylate (1 equiv.) and 4-methoxybenzenesulfonyl chloride (1.5 equiv.) are mixed in 0.8M of Pyridine. Solution is heated to 50° C. for 1.5 hour, and is cooled to room temperature. The mixture is diluted with EtOAc and washed with distilled water, and aqueous layer is extracted with EtOAc. The combined organic layers are washed with distill water three times and Saturated salt solution three times. The solvent is evaporated under reduced pressure, and solid residue is recrystallized with EtOAc and hexane. Product is colorless crystal. Isolated yield: 74%

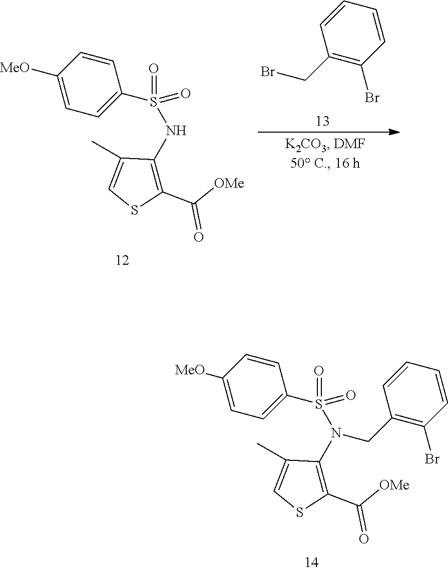 [SMILES: COC(=O)c1scc(C)c1NS(=O)(=O)c1ccc(OC)cc1 ; BrCc1ccccc1Br ; COC(=O)c1scc(C)c1N(Cc1ccccc1Br)S(=O)(=O)c1ccc(OC)cc1]

General Procedure for Benzylation

methyl 3-((4-methoxyphenyl)sulfonamido)thiophene-2-carboxylate (1 equiv.), 1-bromo-2-(bromomethyl)benzene (1.2 equiv.), and K₂CO₃ (3 equiv.) are mixed in 0.3M of DMF. Solution is heated to 50° C. for 16 hours. Reaction mixture is cooled to room temperature after 18 hours. The mixture is diluted with EtOAc and washed with distilled water, and aqueous layer is extracted with EtOAc. The combined organic layers are washed with distill water three times, saturated salt solution three times, and dried over Na₂SO₄ The solvent is evaporated under reduced pressure, and solid residue is recrystallized with EtOAc and hexane. The final product is colorless crystal. Isolated yield: 73%

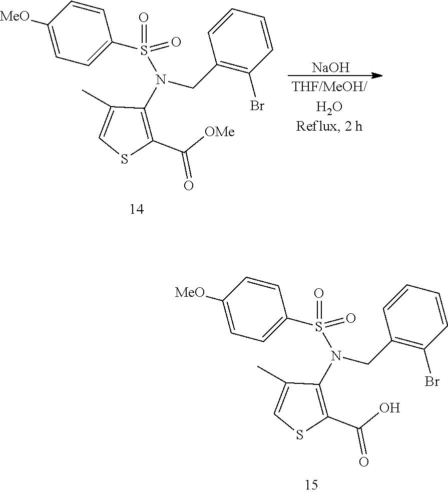

General Procedure for Saponification

methyl 3-((N-(2-bromobenzyl)-4-methoxyphenyl)sulfonamido)-4-methylthiophene-2-carboxylate (1 equiv.) and NaOH powder (5 equiv.) was mixed and dissolved with THF, water, and MeOH (2:1:1 respectively, 0.1M overall). The mixture is heated under reflux, then cooled to room temperature after 2 hours for completion, TLC is used to monitor the reaction. The mixture is treated with 1M HCl solution until pH reaches 1. The mixture is diluted with distilled water and extracted with DCM two times. The combined organic layers are washed with water three time, saturated salt solution two times, and dried over Na₂SO₄. The solvent is evaporated under reduced pressure, and residue is recrystallized with EtOAc and hexane. The final product is colorless solid. Isolated yield: 84%

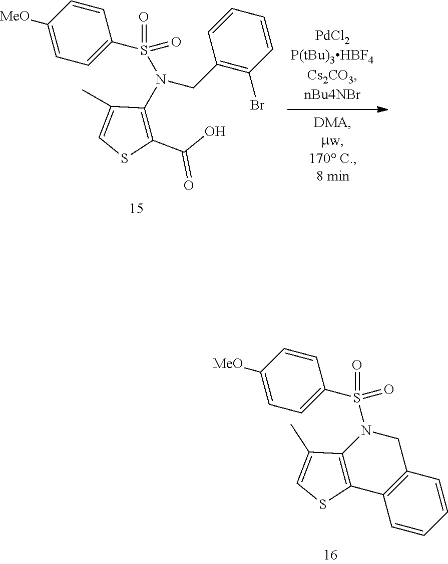 [SMILES: COc1ccc(S(=O)(=O)N(Cc2ccccc2Br)c2c(C)csc2C(=O)O)cc1 ; COc1ccc(S(=O)(=O)N2Cc3ccccc3-c3scc(C)c32)cc1]

General Procedure for Decarboxylative Cross-Coupling

3-((N-(2-bromobenzyl)-4-methoxyphenyl)sulfonamido)-4-methylthiophene-2-carboxylic acid (1 equiv.) in DMA (0.1M) was added to an oven-dried microwave vial contain PdCl₂ (0.1 equiv.), P(tBu)₃.HBF₄ (0.2 equiv.), nBu₄NBr (0.15 equiv.), and Cs₂CO₃ (3 equiv.). The mixture is heated under microwave radiation at 170° C. for 8 min, then cooled to room temperature, and diluted with EtOAc. The mixture is washed with distilled water, and aqueous layer is extracted with EtOAc. The combined layers are washed with saturated NaHCO₃, distilled water, saturated NaCl solution, and dried over Na₂SO₄. The solvent is evaporated under reduced pressure. The product is purified by column chromatography and recrystallization from CHCl₃ and MeOH. The final product is colorless crystal. Isolated yield: 76%.

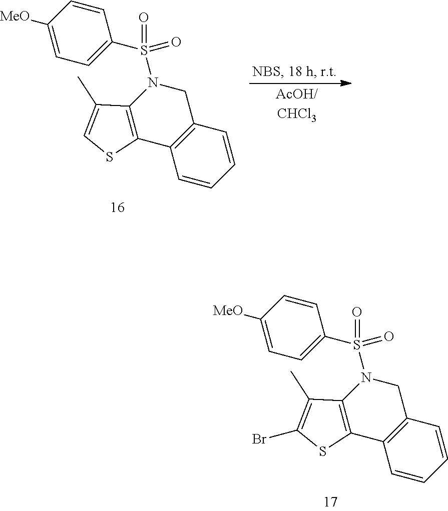 [SMILES: COc1ccc(S(=O)(=O)N2Cc3ccccc3-c3scc(C)c32)cc1 ; COc1ccc(S(=O)(=O)N2Cc3ccccc3-c3sc(Br)c(C)c32)cc1]

General Procedure for Bromination

4-((4-methoxyphenyl)sulfonyl)-3-methyl-4,5-dihydrothieno[3,2-c]isoquinoline (1 equiv.) and NBS (1.1 equiv.) are mixed with CHCl₃ (0.1M) in an ember vial, and mixture is placed in ice bath follow by 1 v/v % AcOH. The reaction is slowly return to room temperature and run for 18 hours. The mixture is diluted with EtOAc then washed with distilled water, and aqueous layer is extracted with EtOAc. The combined organic layer is washed with distilled water one time, saturated salt solution one time, and dried over Na₂SO₄. The solvent is evaporated under reduced pressure. The product is purified by column chromatography. The product is colorless solid. Isolated yield: 71%

### Example 8: C75 Targets Ch-TOG/CKAP5

C75 targets a protein, a structural component or enzyme that regulates the centrosomes responsible for assembly and organization of the mitotic spindle. The target is likely differentially expressed and/or functionally required in cancer cells in comparison to non-cancer cells.

### GFP-Tagged Ch-TOG Localization

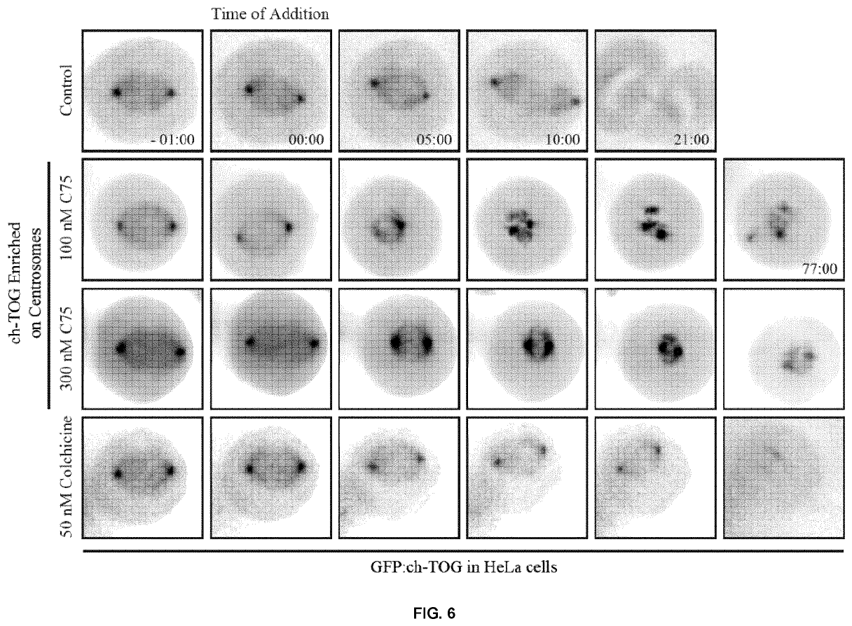

As shown in FIG. 2L and FIG. 6, in HeLa cells expressing SiR-tubulin or GFP-tagged ch-TOG (CKAP5), which localizes to centrosomes, the addition of 100-300 nM of C75 causes the spindle poles to collapse together in metaphase cells within 1-2 minutes. The spindles recover, but become uneven or multipolar. The ability of microtubules to recover to form a spindle in the presence of C75 shows that in cells C75 targets a regulator of centrosomes vs. microtubules.

ch-TOG contains multiple TOG domains that bind to tubulin dimers, and a microtubule-binding domain that binds to the microtubule lattice. If C75 were to bind and inhibit one or more of the TOG domains, this would shift its affinity onto microtubules, while inhibiting the microtubule domain would cause ch-TOG to become cytosolic. As shown in FIG. 6, GFP-tagged ch-TOG becomes enriched at the centrosomes where microtubules are highly concentrated in mitotic HeLa cells after C75 treatment in comparison to non-treated cells, consistent with C75 possibly binding to one or more of the TOG domains.

### Ch-TOG RNAi

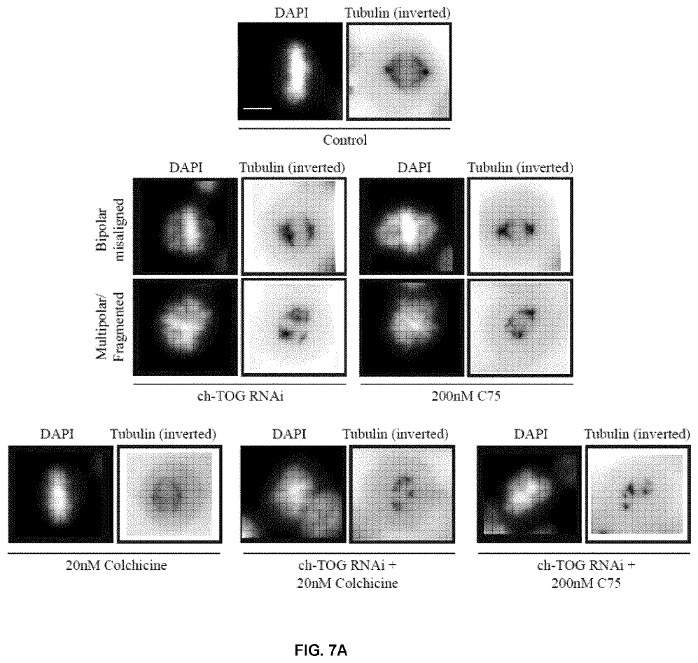

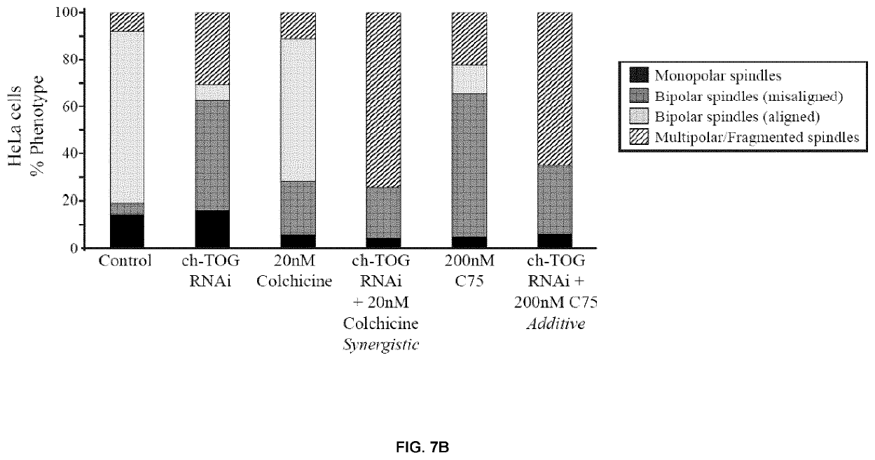

The phenotypes of C75-treated cells are reminiscent of phenotypes caused by ch-TOG (CKAP5), a protein known to regulate microtubule polymerization and mitotic spindle assembly. As shown in FIG. 7A, the mitotic spindle phenotypes observed in HeLa cells after treatment with C75 are similar to phenotypes caused by ch-TOG RNAi.

As shown in FIG. 7B, C75 has an additive effect with ch-TOG RNAi for the fragmented spindle phenotype, but colchicine is synergistic with ch-TOG RNAi. Since RNAi does not knock down endogenous protein completely, this supports that C75 could be targeting ch-TOG, because one would expect the phenotype to get worse, but not more than via additive amounts.

### MCAK RNAi

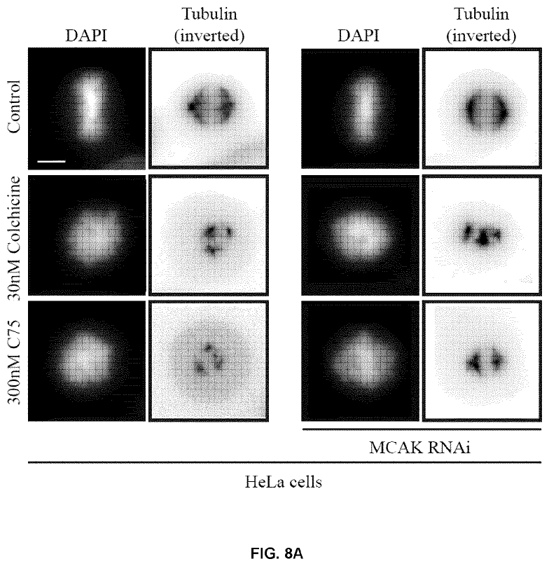

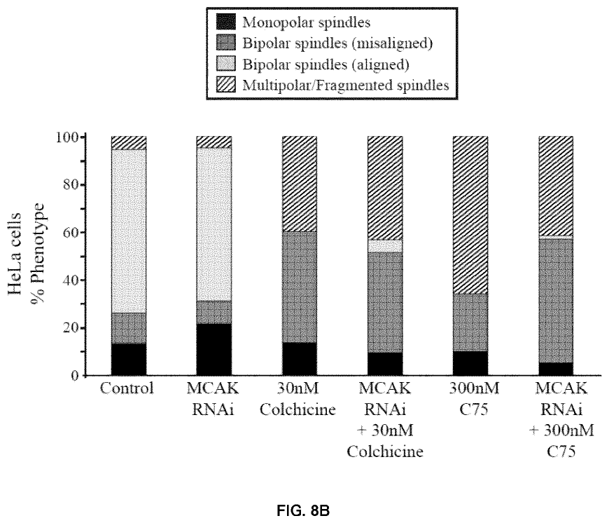

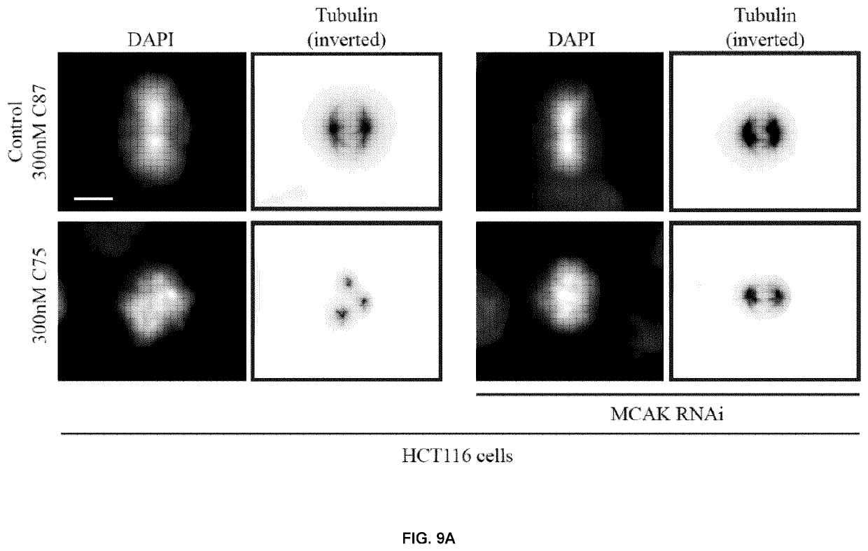

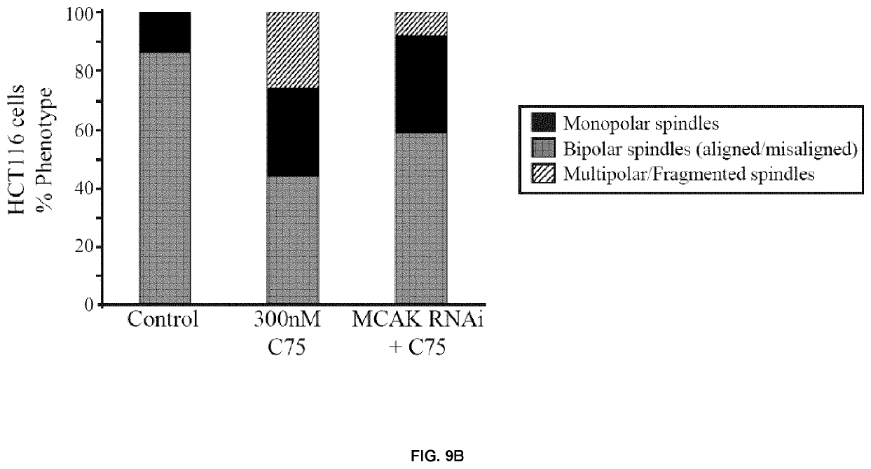

Prior studies showed that the fragmented/multipolar spindle phenotypes caused by ch-TOG RNAi can be suppressed by MCAK RNAi. As shown in FIGS. 8 and 9, we also observed partial suppression of the spindle phenotypes caused by C75 via MCAK depletion, supporting that C75 could target ch-TOG or a component of the pathway.

FIGS. 8 and 9, MCAK RNAi+C75 vs. DMSO control in HeLa (FIG. 8) and HCT116 (FIG. 9) cells shows the proportion of spindle phenotypes. It was observed that the proportion of multipolar spindles caused by C75 are decreased after MCAK depletion.

As shown in FIG. 8, the fragmented spindle phenotype caused by C75, but not colchicine, can be partially suppressed by MCAK RNAi in HeLa cells. Since this is unique to C75, and ch-TOG RNAi was previously shown to be suppressed (partially) by MCAK RNAi, supporting C75's role in targeting ch-TOG or a component of the pathway.

### Example 9: Conservation of Molecular Target of C75

It was found that the molecular target of C75 is conserved in Drosophila. Using S2 cells stably expressing GFP-tagged tubulin, adding 300 nM C75 to live cells caused spindle phenotypes similar to what was observed in mammalian cells. Varying concentrations of C75 in Drosophila S2 cells vs. colchicine were added to determine if the molecular target is conserved. It was found that the cells respond similar to HeLa cells, supporting that the target is conserved. This will permit using Drosophila proteins for binding studies in vitro, and genetics-based tools for further study of the target.

### Example 10: Prophetic Example—C75 Targeting Aurora A Kinase or TPX2

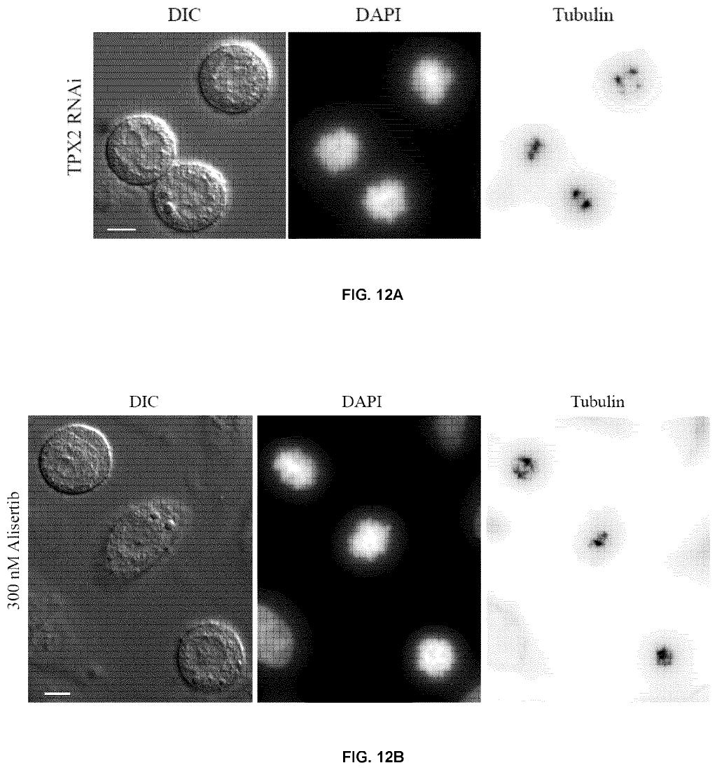

Aurora A kinase regulates the function of multiple proteins that control mitotic spindle assembly, including TPX2, and a complex that comprises ch-TOG, TACC3 and clathrin. The mitotic phenotypes caused by loss of Aurora A kinase function via RNAi or inhibition share similarities to what we observe for C75, as shown in FIG. 12B. TPX2 mediates microtubule nucleation from the centrosomes, and regulates Aurora A kinase, and its depletion causes spindle phenotypes similar to those caused by inhibition of Aurora A kinase or loss of ch-TOG as shown in FIG. 7A and FIG. 12B.

Aurora A kinase inhibition+C75 vs. colchicine in HeLa and HCT116 cells with images and graphs showing the proportion of spindle phenotypes will be assessed. As above, we will compare phenotypes, and determine if C75 enhances/synergizes Aurora A kinase phenotypes, which would rule it out as a target. We will use the Aurora A kinase inhibitor Alisertib, which has been shown to have selectivity for Aurora A kinase, with affinity in the low nanomolar range (˜1 nM in vitro). We will use colchicine as a control, since it should show enhancement.

TPX2 RNAi+C75 vs. DMSO and vs. colchicine in HeLa and HCT116 cells with images and graphs showing the proportion of spindle phenotypes. As above, we will compare phenotypes, and determine if C75 enhances/synergizes TPX2 phenotypes, which would rule it out as a target. As above, we are using colchicine as a control, since it should show enhancement.

### Example 11: Prophetic Example—Cold Treatment Experiments

C75 and its potential target will be further characterized by performing cold treatments of HeLa cells to collapse microtubules followed by recovery by temperature upshift in C75 vs. colchicine or other tubulin-targeting drugs. If C75 targets tubulin, the effect on microtubules should be similar to other tubulin-targeting drugs.

### Example 12: Prophetic Example—In Vitro Binding Studies

To further verify the target, we will perform in vitro binding studies. We have constructs for recombinant expression of the ch-TOG domains from Drosophila and human ch-TOG and have begun expressing and purifying these proteins. We will determine if there are spectral shifts after adding C75 to these purified protein fragments. We also will try DARTs to determine if C75 can protect the various protein fragments from degradation via proteases, which would support binding. We will do similar experiments with other recombinant proteins (e.g. TPX2, tubulin).

### Example 13: Prophetic Example—Determination of Binding Partners

We will use a modified compound, functionalized at a site that does not perturb binding affinity, to couple to a solid matrix for pulldown experiments using cell lysates. This will permit to identify potential binding partners based on affinity, which will be determined via mass spectrometry.

HCT116 cells, colorectal adenocarcinoma, were particularly sensitive to C75. ch-TOG/CKAP5, a microtubule polymerase, is highly overexpressed in these cells, and counteracts the function of MCAK, a microtubule depolymerase, to regulate spindle length during mitosis. Indeed, C75 phenocopies ch-TOG knockdown, and similar to ch-TOG knockdown, the spindle phenotypes caused by C75 can be suppressed by MCAK RNAi. Changes in endogenous ch-TOG localization in the presence of C75 using ch-TOG antibodies were also observed. Despite causing spindle phenotypes, ch-TOG becomes more highly enriched on the centrosomes and microtubules after C75 treatment. This suggests that C75 could bind to at least one of the TOG domains required for binding to free tubulin dimers, causing an increase in ch-TOG affinity to the polymerized microtubules. It is expected, without being bound to this theory that ch-TOG is the molecular target of C75, and it will be determined if other cancer cells that are more sensitive to C75 also have high levels of ch-TOG, making it a suitable biomarker for a subset of highly progressive cancers. Similar logic applies to the other candidates, such as Aurora A kinase or TPX2.

### Example 14: C75 Targets Tubulin

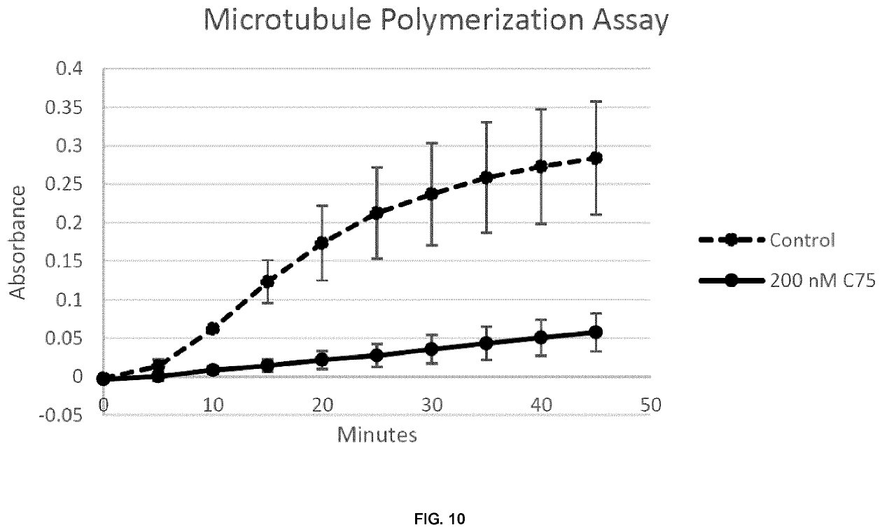

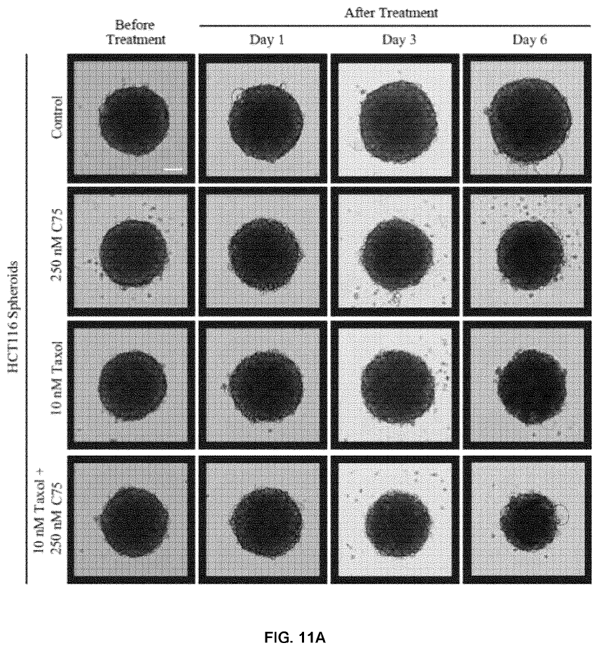

As shown in FIG. 10, C75 prevents microtubule polymerization in vitro, suggesting that it can bind directly to tubulin. Purified free alpha and beta tubulin dimers were added at a concentration of 1.33 g/4 μL to a 20% glycerol buffer and allowed to polymerize in the presence of Mg²⁺ and GTP for 35 minutes at 37° C. While DMSO had no effect on microtubule polymerization, 200 nM of C75 prevented polymerization. For comparison, other anti-cancer drugs that have been shown to bind tubulin and decrease polymerization (e.g. colchicine and vinblastine) cause a similar effect at ˜5 μM. This supports that C75 binds tubulin at a different site compared to other microtubule-depolymerizing drugs.

### Example 15: Prophetic Example—ASPM

C75 could also target ASPM (abnormal spindle-like microcephaly-associated) protein. Asp (abnormal spindle protein), which is conserved and functions in human and Drosophila cells to focus the poles of the mitotic spindle^{[38-42]}. It localizes to the minus ends of microtubules at the poles as well as centrosomes, and its depletion causes displacement of gamma tubulin ring complexes and disorganized spindles, a phenotype that is consistent with what is observed with C75^{[38-41]}. In addition, Asp can nucleate microtubules in vitro and has a microtubule cross-linking domain^{[38,41]}. It is required during prometaphase-metaphase, the time at which C75 causes cell cycle arrest^{[38,41]}.

### Example 16: Prophetic Example—Cdk5Rap2

A recent study found that Asp functions redundantly with Cdk5rap2 (also called CEP215), another potential target of C75 that is conserved in Drosophila^{[42]}. Cdk5rap2/CEP215 localizes to the pericentriolar material (PCM) of centrosomes where it tethers gamma-tubulin ring complexes, and its depletion causes their displacement giving rise to disorganized mitotic spindles, which is similar to what is observed after treatment with C75^{[43-45}. Cancer cells with amplified/fragmented centrosomes may be sensitive to loss of Cdk5rap2 function, because it forms a complex with HSET, a protein that functions to cluster centrosomes^{[45]}.

The embodiments of paragraphs [0036] to [00232] of the present disclosure are presented in such a manner in the present disclosure so as to demonstrate that every combination of embodiments, when applicable can be made. These embodiments have thus been presented in the description in a manner equivalent to making dependent claims for all the embodiments that depend upon any of the preceding claims (covering the previously presented embodiments), thereby demonstrating that they can be combined together in all possible manners. For example, all the possible combination, when applicable, between the embodiments of paragraphs [0036] to [00232] and the methods, processes, uses, compounds and compositions of paragraphs [0006] to [0035] are hereby covered by the present disclosure.

### REFERENCES

1. Hay, M., Thomas D. W., Craighead J. L., Economides C., and Rosenthal, J. 2014. Clinical development success rates for investigational drugs. Nature Biotech. 32: 40-51.
2. Pearce, B. C., Sofia, M. J., Good, A. C., Drexler, D. M., and Stock, D. A. 2006. An empirical process for the design of high-throughput screening deck filters. J. Chem. Inf. Model. 46: 1060-1068.
3. Baell, J. B., and Holloway, G. A. 2010. New substructure filters for removal of pan assay interference compounds (PAINS) from screening libraries and for their exclusion in bioassays. J. Med. Chem. 53: 2719-2740.
4. Lagorce, D., Sperandio, O., Galons, H., Miteva, M. A., and Villoutreix, B. O. 2008. FAF-Drugs2: Free ADME/tox filtering tool to assist drug discovery and chemical biology projects. BMC Bioinformatics 9: 396.
5. Kazius, J., McGuire, R., and Bursi, R. 2005. Derivation and validation of toxicophores for mutagenicity prediction. J. Med. Chem. 48: 312-320.
6. Bruns, R. F., and Watson, I. A. 2012. Rules for identifying potentially reactive or promiscuous compounds. J. Med. Chem. 55: 9763-9772.
7. Mukherjee, S., and Pal., M. 2013. Quinolines: a new hope against inflammation. Drug Discovery Today 18: 389-398.
8. Mukherjee, S., and Pal., M. 2013. Medicinal chemistry of quinolines as emerging anti-inflammatory agents: an overview. Curr Med Chem. 20: 4386-4410.
9. Beccalli, E. M. Broggini, G. Martinelli, M., and Sottocornola, S. 2008. Microwave-assisted intramolecular cyclization of electron-rich heterocycle derivatives by a palladium-catalyzed coupling reaction. Synthesis 1: 136-140.
10. Wong, N. W. Y, and Forgione, P. 2012. A one-pot double C—H activation palladium-catalyzed route to a unique class of highly functionalized thienoisoquinolines Org. Lett. 14: 2738-2741.
11. Chen, F. Wong, N. W. Y., and Forgione, P. 2014. One-pot tandem palladium-catalyzed decarboxylative cross-coupling and C—H activation route to thienoisoquinolines. Adv Synth Cat. 365: 1725-1730.
12. Chan, K. S., Koh, C. G., and Li, H. Y. 2012. Mitosis-targeted anti-cancer therapies: where they stand. Cell Death Dis. 3: e411; doi:10.1038/cddis.2012.148.
13. Parker, A. L., Kavallaris, M., and McCarroll, J. A. 2014. Microtubules and their role in cellular stress in cancer. Front. Oncol. 4: 1-19.
14. Mogilner, A., and Craig, E. 2010. Towards a quantitative understanding of mitotic spindle assembly and mechanics. J. Cell Science 123: 3435-3445.
15. Sacristan, C., and Kops, G. J. 2015. Joined at the hip: kinetochores, microtubules, and spindle assembly checkpoint signaling. Trends Cell Biol. 25: 21-28.
16. Topham, C. H., and Taylor, S. S. 2013. Mitosis and apoptosis: how is the balance set? Curr. Opin. Cell Biol. 25: 780-785.
17. Burgess, A., Rasouli, M., and Rogers, S. 2014. Stressing mitosis to death. Front. Oncol. 4: 140. Doi: 10.3389/fonc.2014.00140.
18. Zasadil, L. M., Andersen, K. A., Yeum, D., Rocque, G. B., Wilke, L. G., Tevaarwerk, A. J., Raines, R. T., Burkard, M. E., and Weaver, B. A. 2014. Cytotoxicity of paclitaxel in breast cancer is due to chromosome missegregation on multipolar spindles. Science Translational Medicine 6:229ra43.
19. Hinchcliffe, E. H. 2014. Centrosomes and the art of mitotic spindle maintenance. Int. Rev. Mol. Cell Mol. Biol. 313: 179-217.
20. Gergely, F., and Basto, R. 2008. Multiple centrosomes: together they stand, divided they fall. Genes Dev. 22: 2291-2296.
21. Godinho, S. A., and Pellman, D. 2014. Causes and consequences of centrosome abnormalities in cancer. Philos. Trans. R. Soc. Lond. B Biol. Sci. 369: 20130467.
22. Ogden, A., Rida, P. C., and Aneja, R. 2012. Let's huddle to prevent a muddle: centrosome declustering as an attractive anticancer strategy. Cell Death Differ. 19: 1255-1267.
23. Chan, J. Y. 2011. A clinical overview of centrosome amplification in human cancers. Int. J. Biol. Sci. 7: 1122-1144.
24. Kawamura, E., Fielding, A. B., Kannan, N., Balgi, A., Eaves, C. J., Roberge, M., and Dedhar, S. 2013. Identification of novel small molecule inhibitors of centrosome clustering in cancer cells. Oncotarget 4: 1763-1776.
25. Eke, I., Leonhardt, F., Storch, K., Hehlgans, S., and Cordes, N. 2009. The small molecule inhibitor QLT0267 radiosensitizes squamous cell carcinoma cells of the head and neck. PLoS One 4: e6434.
26. Fielding, A. B., Lim, S., Montgomery, K., Dobreva, I., and Dedhar, S. 2011. A critical role of integrin-linked kinase, ch-TOG and TACC3 in centrosome clustering in cancer cells. Oncogene 30: 521-534.
27. Yang, B. et al. 2014. Discovery of potent KIFC1 inhibitors using a method of high-throughput synthesis and screening. J. Med. Chem. 57: 9958-9970.
28. Li, Y., Lu, W., Chen, D., Boohaker, R. J., Zhai, L., Padmalayam, I., Wennerberg, K., Xu, B., and Zhang, W. 2015. KIFC1 is a novel potential therapeutic target for breast cancer. Cancer Biol. Ther. 16: 1316-1322.
29. Zhang, W. et al. 2016. Discovery of a novel inhibitor of kinesin-like protein KIFC1. Biochem. J. 473: 1027-1035.
30. Akhmanova, A., and Steinmetz, M. O. 2015. Control of microtubule organization and dynamics: two ends in the limelight. Nat. Rev. Mol. Cell Biol. 16: 711-726.
31. Luconi, M. and Mannelli, M. 2012. Xenograft models for preclinical drug testing: Implications for adrenocortical cancer. Molecular and Cellular Endocrinology 351: 71-77.
32. Hollingshead, M. G. 2008. Antitumor efficacy testing in rodents. J. Natl. Cancer Inst. 100: 1500-1510.
33. Fiebig, H. H., Maier, A., Burger A. M. 2004. Clonogenic assay with established human tumour xenografts: correlation of in vitro to in vivo activity as a basis for anticancer drug discovery. European J. Cancer 40: 802-820.
34. Friedrich, J., Seidel, C., Ebner, R. and Kunz-Schughart, L. A. 2009. Spheroid-based drug screen: considerations and practical approach. Nature protocols. 4: 309-324.
35. Froehlich, K., Haeger, J., Heger, J., Pastuschek, J., Photini, S. M., Yan, Y., Lupp, A., Pfarrer, C., Mrowka, R., Schleussner, E., Markert, U. R. and Schmidt, A. 2016. Generation of multicellular breast cancer tumor spheroids: comparison of different protocols. J. Mammary Gland Biol. Neoplasia 21:89-98.
36. Zhang, Q., Re, Ko, N., Kwon, Oh, J. Recent advances in stimuli-responsive degradable block copolymer micelles: synthesis and controlled drug delivery applications. Chem. Commun. 48, 7542-7552.
37. Hong, S. H., Larocque, K., Jaunky, D., Piekny, A., and Oh, J. 2018. Dual disassembly and biological evaluation of enzyme/oxidation-responsive polyester-based nanoparticulates for tumor-targeted delivery. Colloids and Surfaces B: Biointerfaces. 172:608-617.
38. do Carmo Avides, M. and Glover, D. M. 1999. Abnormal spindle protein, Asp, and the integrity of mitotic centrosomal microtubue organizing centers. Science 283: 1733-1735.
39. Wakefield, J. G., Bonaccorsi, S. and Gatti, M. 2001. The Drosophila protein asp is involved in microtubule organization during spindle formation and cytokinesis. J. Cell Biol. 153: 637-648.
40. Morales-Mulia, S. and Scholey, J. M. 2005. Spindle organization in Drosophila S2 cells by dynein, abnormal spindle protein (Asp), and KLP10A. Mol. Cell. Biol. 16: 3176-3186.
41. Ito, A. and Goshima, G. 2015. Microcephaly protein Asp focuses the minus ends of spindle microtubules at the pole and within the spindle. J. Cell Biol. 211: 999-1009.
42. Tungadi, E. A., Ito, A., Kiyomitsu, T., and Goshima, G. 2017. Human microcephaly ASPM protein is a spindle pole-focusing factor that functions redundantly with CDK5RAP2. J. Cell Sci. 130: 3676-3684.
43. Graser, S., Stierhof, Y. D., and Nigg, E. A. 2007. Cep68 and Cep215 (Cdk5rap2) are required for centrosome cohesion. J. Cell Sci. 120: 4321-4331.
44. Fong, K. W., Choi, Y. K., Rattner, J. B., and Qi, R. Z. 2008. CDK5RAP2 is a pericentriolar protein that functions in centrosomal attachment of the {gamma}-tubulin ring complex. Mol. Biol. Cell 19: 115-125.
45. Chavali, P. L., Chandrasekaran, G., Barr, A. R., Tatrai, P., Taylor, C., Papachristou, E. K., Woods, C. G., Cahavli, S., and Gergely, F. 2016. A CEP215-HSET complex links centrosomes with spindle poles and drives centrosome clustering in cancer. Nat. Commun. 7: 11005.

1. A method for targeting tubulin, ch-TOG, Aurora A kinase, TPX2, Cdk5rap2 and/or ASPM expressed in a cancer cell and selectively inhibiting growth therein, comprising exposing said cancer cell to a compound of Formula I: 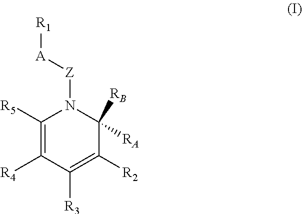
wherein
A is a C₆-C₁₂ aryl or a three- to seven-membered aromatic heterocycle;
Z is SO, SO₂, CO or CH₂;
R_{A} and R_{B} are each independently H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₁ is H, C₁-C₆ alkyl, C₁-C₆ alkoxy, C₁-C₆ hydroxyalkyl, C₁-C₆ alkythio, C₁-C₆ thioalkyl, C₁-C₆ haloalkyl, C₁-C₆ sulfonylakyl, C₁-C₆ aminoalkyl, C₁-C₆ alkylamino, CN, CF₃, CF₂H, CFH₂, F, Cl, Br, I, a C₆-C₁₂ aryl or a three- to seven-membered aromatic heterocycle;
R₂ and R₃ are joined together to form a C₆-C₁₂ aryl or a three- to seven-membered aromatic heterocycle; and
R₄ and R₅ are joined together to form a C₆-C₁₂ aryl or a three- to seven-membered aromatic heterocycle,
R₁, R_{A}, R_{B} said C₆-C₁₂ aryl and said three- to seven-membered aromatic heterocycle being each independently unsubstituted or substituted with at least one substituent chosen from C₁-C₆ alkyl, C₁-C₆ alkoxy, C₆-C₁₂ aryl, three- to seven-membered aromatic heterocycle, C₁-C₆ hydroxyalkyl, C₁-C₆ alkythio, C₁-C₆ thioalkyl, C₁-C₆ sulfonylakyl, C₁-C₆ aminoalkyl, C₁-C₆ alkylamino, CN, NO₂, 4,5-dioxoyl, NH₂, CF₃, CF₂H, CFH₂, F, Cl, Br, I, OH, CHO, COOH and COOR_{C}, wherein R_{C} is a C₁-C₆ alkyl,
or a pharmaceutically acceptable salt, solvate or prodrug thereof,
wherein the cancer cell expresses Cdk5rap2.

2-3. (canceled)

4. A method for targeting tubulin, ch-TOG, Aurora A kinase, TPX2, Cdk5rap2 and/or ASPM expressed in a cancer cell and selectively inhibiting growth therein, comprising exposing said cancer cell to a combination of a compound of Formula I and an anti-cancer agent and/or an anti-mitotic agent, wherein said anti-cancer agent is a taxane, a vinca alkaloid or a colchicine-site binder.

5. A method for targeting tubulin, ch-TOG, Aurora A kinase, TPX2, Cdk5rap2 and/or ASPM expressed in a cancer cell and inhibiting growth therein, comprising exposing said cancer cell to a synergistic combination of a compound of Formula I and an anti-cancer agent and/or an anti-mitotic agent, wherein said combination more than additively inhibits growth of said cancer cell, wherein the anti-mitotic agent is nocodazole.

6-30. (canceled)

31. The method of claim 4, wherein said anti-cancer agent is a non-mitotic anti-cancer agent, wherein said non-mitotic anti-cancer agent is doxorubicin, an anthracycline, an alkylating drug or an antimetabolite.

32. (canceled)

33. The method of claim 4, wherein said taxane is paclitaxel, cabazitaxel, or docetaxel.

34. The method of claim 4 wherein said vinca alkaloid is vinblastine, vincristine, vindesine, or vinorelbine.

35. The method of claim 4, wherein said colchicine-site binder is colchicine, a combrestatin or podophyllotoxin.

36-71. (canceled)
